SEQUENCE LISTING (18 sequences)

SEQ ID NO: 1 - DNA, 4107 bp, Homo sapiens
gtcgagcgggagcagaggaggcgagggaggagggccagagaggcagttggaagatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatagtgtaataattgtgcaggtacaggaattgttccaccagcattaggaactttagcatgtcaaaatgaatgtttacttgtgaactcgatagagcaaggaaaccagaaaggtgtaatatttataggttggtaaaatagattgtttttcatggataatttttaacttcattatttctgtacttgtacaaactcaacactaacttttttttttttaaaaaaaaaaaggtactaagtatcttcaatcagctgttggtcaagactaactttcttttaaaggttcatttgtatgataaattcatatgtgtatatataattttttttgttttgtctagtgagtttcaacatttttaaagttttcaaaaagccatcggaatgttaaattaatgtaaagggacagctaatctagaccaaagaatggtattttcacttttctttgtaacattgaatggtttgaagtactcaaaatctgttacgctaaacttttgattctttaacacaattatttttaaacactggcattttccaaaactgtggcagctaactttttaaaatctcaaatgacatgcagtgtgagtagaaggaagtcaacaatatgtggggagagcactcggttgtctttacttttaaaagtaatacttggtgctaagaatttcaggattattgtatttacgttcaaatgaagatggcttttgtacttcctgtggacatgtagtaatgtctatattggctcataaaactaacctgaaaaacaaataaatgctttggaaatgtttcagttgctttagaaacattagtgcctgcctggatccccttagttttgaaatatttgccattgttgtttaaatacctatcactgtggtagagcttgcattgatcttttccacaagtattaaactgccaaaatgtgaatatgcaaagcctttctgaatctataataatggtacttctactggggagagtgtaatattttggactgctgttttccattaatgaggagagcaacaggcccctgattatacagttccaaagtaataagatgttaattgtaattcagccagaaagtacatgtctcccattgggaggatttggtgttaaataccaaactgctagccctagtattatggagatgaacatgatgatgtaacttgtaatagcagaatagttaatgaatgaaactagttcttataatttatctttatttaaaagcttagcctgccttaaaactagagatcaactttctcagctgcaaaagcttctagtctttcaagaagttcatactttatgaaattgcacagtaagcatttatttttcagaccatttttgaacatcactcctaaattaataaagtattcctctgttgctttagtatttattacaataaaaagggtttgaaatatagctgttctttatgcataaaacacccagctaggaccattactgccagagaaaaaaatcgtattgaatggccatttccctacttataagatgtctcaatctgaatttatttggctacactaaagaatgcagtatatttagttttccatttgcatgatgtttgtgtgctatagatgatattttaaattgaaaagtttgttttaaattatttttacagtgaagactgttttcagctctttttatattgtacatagtcttttatgtaatttactggcatatgttttgtagactgtttaatgactggatatcttccttcaacttttgaaatacaaaaccagtgttttttacttgtacactgttttaaagtctattaaaattgtcatttgacttttttctgttaaaaaaaaaaaaaaaaaaaa

SEQ ID NO: 2 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 3 - DNA, 19 bp, Artificial Sequence
  Description of Artificial Sequence Synthetic Oligonucleotide
cttgtacgacgaagacgac

SEQ ID NO: 4 - DNA, 19 bp, Artificial Sequence
  Description of Artificial Sequence Synthetic Oligonucleotide
ggccacggataggtccata

SEQ ID NO: 5 - DNA, 19 bp, Artificial Sequence
  Description of Artificial Sequence Synthetic Oligonucleotide
catagacacgctggaacag

SEQ ID NO: 6 - DNA, 19 bp, Homo sapiens
cttgtacgacgaagacgac

SEQ ID NO: 7 - DNA, 64 bp, Artificial Sequence
  Description of Artificial Sequence Synthetic primer
gatcccccttgtacgacgaagacgacttcaagagagtcgtcttcgtcgtacaagtttttggaaa

SEQ ID NO: 8 - DNA, 64 bp, Artificial Sequence
  Description of Artificial Sequence Synthetic primer
agcttttccaaaaacttgtacgacgaagacgactctcttgaagtcgtcttcgtcgtacaagggg

SEQ ID NO: 9 - DNA, 20 bp, Homo sapiens
ggccacggataggtccatat

SEQ ID NO: 10 - DNA, 64 bp, Artificial Sequence
  Description of Artificial Sequence Synthetic primer
gatccccggccacggataggtccatattcaagagatatggacctatccgtggcctttttggaaa

SEQ ID NO: 11 - DNA, 64 bp, Artificial Sequence
  Description of Artificial Sequence Synthetic primer
agcttttccaaaaaggccacggataggtccatatctcttgaatatggacctatccgtggccggg

SEQ ID NO: 12 - DNA, 19 bp, Homo sapiens
catagacacgctggaacag

SEQ ID NO: 13 - DNA, 64 bp, Artificial Sequence
  Description of Artificial Sequence Synthetic primer
gatcccccatagacacgctggaacagttcaagagactgttccagcgtgtctatgtttttggaaa

SEQ ID NO: 14 - DNA, 64 bp, Artificial Sequence
  Description of Artificial Sequence Synthetic primer
agcttttccaaaaacatagacacgctggaacagtctcttgaactgttccagcgtgtctatgggg

SEQ ID NO: 15 - DNA, 19 bp, Artificial Sequence
  Description of Artificial Sequence Synthetic primer
agccgcgtctggttctagt

SEQ ID NO: 16 - DNA, 16 bp, Artificial Sequence
  Description of Artificial Sequence Synthetic primer
ggaggctgcagggctg

SEQ ID NO: 17 - DNA, 21 bp, Artificial Sequence
  Description of Artificial Sequence Synthetic primer
tagagggtcaccgcgtctatg

SEQ ID NO: 18 - DNA, 21 bp, Artificial Sequence
  Description of Artificial Sequence Synthetic primer
gggtgcgtggctgcagccagg

(19) United States
(12) Patent Application Publication
(10) Pub. No.: US 2011/0104177 A1
(43) Pub. Date: May 5, 2011

(54) HISTONE DEACETYLASE INHIBITORS, COMBINATION THERAPIES AND METHODS OF USE

(75) Inventors: Stephen Baylin, Baltimore, MD (US); Kevin Pruitt, Shreveport, LA (US)

(73) Assignee: THE JOHNS HOPKINS UNIVERSITY, Baltimore, MD (US)

(21) Appl. No.: 12/521,569

(22) PCT Filed: Dec. 28, 2007
(86) PCT No.: PCT/US07/26474

§ 371 Date: Jan. 10, 2011

Related U.S. Application Data
(60) Provisional application No. 60/877,469, filed on Dec. 28, 2006.
(60) Provisional application No. 60/899,799, filed on Feb. 5, 2007.
(60) Provisional application No. 60/899,986, filed on Feb. 6, 2007.

(51) Int. Cl. A61K 31/713; A61K 31/7105; C07H 21/02; A61K 31/706; A61K 31/455; A61K 31/16; C12Q 1/68; A61K 31/403; A61K 31/366; A61K 39/395; A61K 38/02; A61P 35/00

(52) U.S. Cl. 424/158.1; 514/44 A; 514/44 R; 536/24.5; 514/43; 514/355; 514/575; 435/6; 514/411; 514/455; 514/1.1

(57) ABSTRACT

The invention relates to histone deacetylase (HDAC) inhibitors to treat proliferative diseases. The present invention provides novel class III histone deacetylase inhibitors, in particular SIRT1 inhibitors, to reverse the silencing of hypermethylated genes, in combination with one or more other agents, in proliferative diseases such as cancer. The present invention provides methods of activating genes that are silenced by methylation in a subject by administering a HDAC inhibitor in combination with one or more agents.

### RELATED APPLICATIONS

This application claims the benefit of U.S. Provisional Application No. 60/877,469 filed on Dec. 28, 2006, U.S. Provisional Application No. 60/899,799 filed on Feb. 5, 2007 and U.S. Provisional Application No. 60/899,986 filed on Feb. 6, 2007. The entire contents of the aforementioned applications are hereby incorporated herein by reference.

### FIELD OF THE INVENTION

The invention relates to histone deacetylase (HDAC) inhibitors to treat proliferative diseases. The present invention provides novel class III histone deacetylase inhibitors, in particular SIRT1 inhibitors, to reverse the silencing of hypermethylated genes, in particular tumor suppressor genes, in proliferative diseases such as cancer. The present invention provides methods of activating genes that are silenced by methylation in a subject by administering a HDAC inhibitor in combination with one or more agents.

### BACKGROUND

According to the American Cancer Society, about 1,444,920 new cancer cases were expected to be diagnosed in the year 2007. In 2007 alone, about 559,650 Americans are expected to die of cancer, more than 1500 people a day. Cancer is the second most common cause of death in the United States, and accounts for 1 of every 4 deaths. The 5-year relative survival rate for all cancers diagnosed between the years of 1996 and 2002 is 66%, which is up from 51% in 1975-1977, and reflects progress in diagnosing certain cancers at an earlier stage, and improvements in treatment. (http://www.cancer.org/downloads/STT/CAFF2007PWSecured.pdf).

There is a growing list of tumor suppressor genes (TSGs) and candidate TSGs that are epigenetically silenced in virtually every cancer type, and this silencing has been associated with aberrant promoter DNA methylation [1-3]. In previous studies, silencing of these genes was shown to involve dense hypermethylation of 5′ CpG islands and hypoacetylation of lysine 9 and 14 on histone H3 (H3-K9 and H3-K14, respectively) [4,5]. Moreover, it has been shown that synergistic reactivation of these TSGs can be achieved only when class I/II histone deactylase (HDAC) inhibitors (HDIs) are employed to treat tumor cells after DNA demethylating agents, such as 5-deoxy-azacytidine (DAC), have first induced at least partial promoter demethylation [5,6]. This suggested a dominance of the DNA methylation over deactylation for maintenance of gene silencing [1].

Another important class of HDACs is the NAD⁺-dependent sirtuins, or class III HDACs [7]. The most prominent human family member, SIRT1 (Q96EB6), has been shown to regulate transcriptional repression of mammalian target genes that are either already basally expressed [8] or to regulate transcriptional repression of an integrated Gal4-fusion reporter plasmid [9-11]. The sirtuins have distinct specific inhibitors [12-14] and are not responsive to drugs like trichostatin-A (TSA) or other class I and II HDIs previously used to study promoter-hypermethylated TSGs. At least eight different class I/II HDIs are advancing in different phases of clinical trials for cancer treatment [15,16]; however to date inhibitors of sirtuin deacetylases have not been investigated for such use.

Accordingly, there is a need in the art for new, more effective cancer therapies, and in particular, class III HDIs.

### SUMMARY

The present invention relates generally to class III histone deacetylase (HDAC) inhibitors (HDIs), and in particular cancer genes. Applicants have discovered that combinations of certain types of therapeutic compounds can be used for the treatment of conditions and disorders involving aberrant gene silencing, such as conditions and disorders involving aberrant cell growth, e.g., cancer.

In a first aspect, the invention features a method of activating genes that are silenced by methylation in a subject comprising administering a histone deacetylase (HDAC) inhibitor.

In one embodiment, activating the genes comprises increased gene expression.

In another embodiment, the HDAC inhibitor is administered in combination with one or more agents.

In another particular embodiment, the genes that are silenced by methylation are methylated in the promoter region. In a further embodiment, the methylation is hypermethylation.

In one embodiment, the subject is suffering from a proliferative disease or disorder. In a further embodiment, the proliferative disease or disorder is selected from a neoplasia, myelofibrosis, or proliferative diabetic retinopathy.

In a most preferred embodiment, the invention features a method of activating genes that are silenced by methylation in a subject suffering from a neoplasia, comprising administering a histone deacetylase (HDAC) inhibitor.

In a related embodiment, the neoplasia is a cancer. In a more particular embodiment, the cancer is selected from the group consisting of: breast, ovarian, liver, lung, and prostate cancers.

In a further related embodiment, the cancer comprises genes that are silenced by methylation. In a more particular embodiment, the genes are tumor suppressor genes. In one embodiment, the tumor suppressor genes are selected from the group consisting of: secreted frizzled related proteins, p53, E-cadherin, mismatch repair genes, and cellular retinol binding protein-1 (CRBP-1).

In another aspect, the invention features a method of activating methylation-silenced genes in a subject comprising administering a histone deacetylase (HDAC) inhibitor in combination with one or more agents.

In a particular embodiment, gene activation comprises increased gene expression.

In another aspect, the invention features a method of treating a proliferative disease or disorder comprising administering a histone deacetylase (HDAC) inhibitor in combination with one or more agents.

In one embodiment of any one of the above-mentioned aspects, at least one of the one or more agents is an inhibitor of epigenetic silencing. In another embodiment of at least one of the above-mentioned aspects, the HDAC inhibitor is a class III HDAC inhibitor. In a more particular embodiment, the class III HDAC inhibitor is a SIRT1 inhibitor.

In another aspect, the invention features a method of treating a proliferative disease or disorder comprising administering a SIRT1 inhibitor in combination with one or more agents, wherein at least one of the one or more agents is an inhibitor of epigenetic silencing.

In one aspect, the class III HDAC inhibitor is selected from a siRNA, a dsRNA, an shRNA, a ribozyme, an antisense nucleic acid, a retroviral inhibitor, an adenoviral inhibitor, or a small molecule inhibitor. In another aspect, the siRNA inhibits expression of SIRT1.

In another aspect, the invention features a siRNA that inhibits expression of SIRT1 in a cell.

In one embodiment, the siRNA comprises a contiguous sequence of 10-30 bp from the sequence of SEQ ID NO: 1.

In another embodiment, the siRNA according to the above-mentioned aspect is between 19 and 25 bp in length.

In another related embodiment, the siRNA according to the above-mentioned aspect comprises SEQ ID NO: 3, SEQ ID NO: 4 or SEQ ID NO: 5.

In another related aspect according to any one of the above aspects as described herein at least one of the one or more agents is an agent that promotes demethylation.

In a particular embodiment, at least one of the one or more agents is a HDAC inhibitor. In another embodiment, the HDAC inhibitor is selected from the group consisting of an inhibitor of the class of: HDAC I, HDAC II and HDACIII. In still another embodiment the agent is selected from: 5-azadeoxycytodine, nicotinamide, splitomycin, and trichostatin-A.

In a related embodiment according to any one of the above-mentioned aspects, at least one of the one or more agents is a chemotherapeutic agent.

In another aspect, the invention features a method of identifying a SIRT1 inhibitor comprising administering a candidate compound to a cell with one or more genes that are silenced by methylation in vitro; and determining whether gene expression in increased in said cell; wherein increased gene expression compared to untreated cells identifies a SIRT1 inhibitor.

In one embodiment of the method, the SIRT1 inhibitor does not affect gene methylation.

In another embodiment of any one of the above-described aspects, the proliferative disease or disorder is selected from a neoplasia, myelofibrosis, or proliferative diabetic retinopathy.

In a further embodiment, the neoplasia is a cancer. In a related embodiment, the cancer is selected from the group consisting of: breast, ovarian, liver, lung, and prostate cancer. In still a further embodiment, the cancer comprises genes that are silenced by methylation.

In a particular embodiment, the genes are tumor suppressor genes. In a related embodiment, the tumor suppressor genes are selected from the group consisting of: secreted frizzled related proteins, p53, E-cadherin, mismatch repair genes, and cellular retinol binding protein-1 (CRBP-1).

In another aspect the invention features a pharmaceutical composition comprising a siRNA according to any one of the above-mentioned aspects and a pharmaceutically acceptable excipient.

In another aspect the invention features a pharmaceutical composition comprising a SIRT1 inhibitor according to any one of the above-mentioned aspects and a pharmaceutically acceptable excipient

In yet another aspect the invention features a kit for use in a method of activating methylation silenced genes in a subject comprising administering a histone deacetylase (HDAC) inhibitor according to any one of the above-mentioned claims and instructions for use.

In still another aspect the invention features a kit for use in the method of activating methylation-silenced genes in a subject comprising administering a histone deacetylase (HDAC) inhibitor in combination with one or more agents and instructions for use.

In another aspect the invention features a kit for use in a method of treating a proliferative disease or disorder comprising administering a histone deacetylase (HDAC) inhibitor in combination with one or more agents according to any one of the above-mentioned aspects and instructions for use.

All cited patents, patent applications, and references are hereby incorporated by reference in their entireties. In the case of conflict, the present application controls.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

FIG. 1A-1F are a series of panels demonstrating that siRNA knockdown of SIRT1 causes re-expression of epigenetically silenced tumor suppressor genes. Panel (A) shows RNAi-3 is most effective for reduction of SIRT1 in MCF7 cells. Retroviral expression vectors encoding SIRT1 cDNA that produce short hairpin loop RNA targeting either distinct regions of SIRT1 mRNA (RNAi-1, -2, or -3) or a control (ctrl) was used to infect MCF7. Western blot analysis for SIRT1 and b-actin was performed 48 h after two rounds of infection. Panel (B) shows both RNAi-2 and -3 are effective for reduction of SIRT1 protein in MDA-MB-231 cells as described in (A). Panel (C) shows SIRT1 inhibition leads to TSG re-expression in MCF7 cells. RNA was isolated from parallel samples analyzed in (A), and RT-PCR was performed with intron-spanning primers specific for the genes SFRP1 and SFRP2. GAPDH was also analyzed as a control. Only the shRNA (RNAi-3) that caused substantial reduction in SIRT1 protein leads to gene re-expression. Control samples in which no reverse transcriptase was added were analyzed separately, and all were negative for amplification of the indicated genes. Panel (D) shows SIRT1 inhibition leads to TSG re-expression in MDA-MB-231 cells. RTPCR was performed for analysis of the genes SFRP1, SFRP2, and E-cadherin as described in (A). Only the shRNAs (RNAi-2 and -3) that caused substantial reduction in SIRT1 protein lead to gene re-expression. Panel (E) shows that SIRT1 inhibition leads to TSG re-expression in RKO cells. SIRT1 protein reduction by RNAi-3 (top panel) as described in (A) leads to gene re-expression of SFRP1, SFRP2, and MLH1 as described in (C). Panel (F) shows MDA-MB-231 and RKO cells infected with control or RNAi-3 shRNA as described in (A) were selected with puromycin for 3 d, and pooled colonies were harvested for Western blot analysis of protein re-expression that corresponded with the gene reactivation described in (D) and (E).

FIG. 2A-2F are a series of panels demonstrating that pharmacologic and dominant negative inhibition of SIRT1 case re-expression of tumor suppressor genes and synergize with 5-deoxy-azacytidine (DAC) or trichostatin-A (TSA). Panel (A) shows pharmacologic inhibition of SIRT1 causes TSG re-expression. MDAMB-231 cells were treated with 15 mM NIA or 300 lM SPT for 21 h, RNA was isolated, and RT-PCR was performed with introns-spanning primers specific for the indicated genes. Control samples in which no reverse transcriptase was added were analyzed separately, and all were negative for amplification of the indicated genes. Panel (B) shows combined treatment with low doses of Aza and SPT synergizes in the re-expression of TSGs. MDA-MB-231 cells were treated with either 50 nM Aza ( [symbol] ), 100 lM SPT ( [symbol] ) or with both Aza and SPT ( [symbol] [symbol] ), and 34 h later, RTPCR was performed for the indicated genes as described in (A). Panel (C) shows combined treatment with SPT and TSA synergize in the re-expression of genes. MDA-MB-231 cells were treated with 0, 50, 100, or 120 lM SPT alone for 34 h, or the treatment was followed by treatment with 300 nM TSA for 3 h prior to RNA isolation and RT-PCR analysis. Panel (D) shows SIRT1 protein knockdown synergizes with low doses of Aza for gene re-expression. MDA-MB-231 cells were infected with low titers of virus for shRNA specific for SIRT1. Aza (100 nM) was added 24 h prior to RNA isolation, and RT-PCR analysis was performed for the genes SFRP1, SFRP2, and GAPDH as described in (A). Panel (E) shows dominant negative inhibition of SIRT1 leads to TSG re-expression in MCF7 cells. MCF7 cells were infected with virus encoding either pBabe (vec) or the catalytically inactive SIRT1H363Y (HY) mutant, and RT-PCR was performed as described in (A). Panel (F) shows dominant negative inhibition of SIRT1 leads to TSG re-expression and synergizes with TSA and Aza. As shown in the left panel, MDA-MB-231 cells were infected with a control (vec) or mutant SIRT1 virus (HY), and RT-PCR was performed as described in (A). MDA-MB-231 cells were infected with low titers of pBabe or pBabe-SIRT1H363Y retrovirus and subsequently treated with 100 nM Aza for 24 h or with 300 nM TSA for 3 h prior to harvest, and RT-PCR was performed.

FIGS. 3A and 3B are two panels that show SIRT1 inhibition causes TSG re-expression without changing promoter DNA hypermethylation. Panel (A) is a schematic showing the levels of promoter methylation. Panel A shows that TSG re-expression occurs without changes in the methylation profile of multiple clones analyzed for SFRP1 promoter methylation. Parallel samples analyzed in FIG. 1D were subjected to bisulfite sequencing of the SFRP1 promoter from MDA-MB-231 cells stably infected with control vector or RNAi-2 or RNAi-3 retrovirus. Open circles indicate unmethylated cytosines, and closed circles indicate methylated cytosines. Numbers at the bottom show the position of cytosines relative to the transcription start site, which is at position 0, and those with a minus sign (−) are upstream from this start site. The region sequenced encompasses the CpG island in which methylation status correlates with gene expression status. Panel (B) is a reproduction showing tumor suppressor gene re-expression after SIRT1 inhibition. Shown in Panel (B) are MSP analyses of DNA from MDA-MB-231 cells stably expressing vector control, RNAi-2, or RNAi-3 retrovirus. From left to right: (−) PCR Ctrl indicates H2O only; (−) BS ctrl indicates bisulfite-treated H20; ( [symbol] ) M ctrl indicates the cell line in which SFRP1 is partially methylated and SFRP2 and GATA4 are fully methylated; and ( [symbol] ) U ctrl indicates the Tera-2 cell line in which each gene is unmethylated. All remaining lanes are for MDA-MB-231. From left to right: Aza indicates 1 lM Aza (24 h) treatment; Ctrl indicates empty vector infection; RNAi-2 indicates shRNA-2 infection alone; RNAi-3 indicates shRNA-3 infection alone; Aza indicates 1 lM Aza (24 h) treatment of control cells; Ctrl indicates empty vector infection [symbol] vehicle; RNAi-2 indicates shRNA-2 infection b 5 mM NIA treatment; and RNAi-3 indicates shRNA-3 infection [symbol] 5 mM NIA treatment.

FIGS. 4A and 4B are two panels illustrating A) a reproduction and B) a bar graph showing re-expression of epigenetically silenced tumor suppressor genes after SIRT1 inhibition. In Panel (A) RKO cells were infected and stably selected to express short hairpin loop RNA targeting either a region unique to SIRT1 mRNA or a control (ctrl). To inhibit any residual SIRT1 protein, remaining RNAi-expressing cells were treated with 700 lM SPT and control samples were treated with DMSO for 24 h. For comparison, control RNA was isolated from parallel samples from HCT116 cells in which the two genes under study, CRB1 and E-cadherin, do not have promoter DNA hypermethylation and are basally expressed. RKO cells were also treated with 0.5 lM Aza (24 h), and samples were analyzed as described in FIG. 1A; RT-PCR was performed with intron-spanning primers specific for the two genes. GAPDH was also analyzed as a control. Only the shRNA (RNAi-3) that caused substantial reduction in SIRT1 protein leads to gene re-expression. Control samples in which no reverse transcriptase was added were analyzed separately, and all were negative for amplification of the indicated genes. In Panel (B) parallel samples described above were analyzed using real-time quantitative PCR. The level of TSG re-expression induced by Aza treatment or SIRT1 inhibition as described in (A) was compared to levels of expression in HCT116 cells in which the TSGs are basally expressed.

FIG. 5A-5C are reproductions and a bar graph showing that inhibition of SIRT1 causes increases in histone H4-K16 acetylation at the promoter of re-expressed genes. Panel (A) shows that pooled populations of MDA-MB-231 cells stably selected to express RNAi constructs were analyzed via ChIP. These samples were isolated in parallel to those analyzed in FIG. 3B. ChIP was performed with antibodies against SIRT1, acetylated histone H4, lysine 16 (H4-K16), or with no antibody (NAB) controls. Each promoter sequence was amplified by PCR under linear conditions for the genes SFRP1 and E-cadherin. In Panel (B) the average change in SIRT1 localization, acetylation of H4-K16, and acetylation of H3K9 at the SFRP 1 and E-cadherin promoters as measured by ChIP was quantitated for multiple experiments. Error bars indicate the standard deviation for multiple experiments. In Panel (C) SIRT1 localizes to the promoters of silent genes whose DNA is hypermethylated, but not to these same promoters in cells in which the genes are expressed. ChIP was performed with antibodies against SIRT1 in RKO and SW480 colon cancer cells. As shown in the left panel, SIRT1 localizes to the MLH1 promoter in RKO cells in which the gene is silent, but not to the MLH1 promoter in SW480 cells in which it is expressed. As shown in the right panel, SIRT1 localizes to the E-cadherin promoter in RKO cells in which the gene is silent, but not to the E-cadherin promoter in SW480 cells where it is expressed.

FIG. 6A-6D are a series of bar graphs and reproductions showing that SIRT1 inhibition affects phenotypic aspects of cancer cells. In Panel (A) MDA-MB-231 cells were infected for two rounds with RNAi-2 and -3 retrovirus, and puromycin-resistant colonies were counted after 3 d of selection. Error bars indicate standard deviation from the average of three experiments. In Panel (B) RKO cells were transfected with 500 ng of pGL3-OT, a TCFLEF-responsive reporter, or pGL3-OF, a negative control with a mutated TCF-LEF binding site in combination with 10 ng of pRL-CMV vector. Twenty-four hours post-transfection, cells were treated with either vehicle (DMSO) control or with 700 lM SPT for 24 h. Firefly luciferase activity was measured and normalized to the Renilla luciferase activities. In Panel (C), as described in (A), pooled populations of MDA-MB-231 cells stably expressing RNAi-2 or RNAi-3 were harvested, protein concentrations were determined, and Western blot analysis was performed. An antibody that specifically recognizes the unphosphorylated (active) form of b-catenin was used, and on the same blot, b-actin was probed to ensure equal loading. In Panel (D) Western blot analysis was performed on RKO cells expressing control or SIRT1 RNAi. Antibodies against SIRT1, phospho-GSK3b (inactive), cyclin D1, p27, and b-actin were used for Western blotting. On the same blot, b-actin was probed to ensure equal loading.

FIG. 7 is a panel showing the re-expression of tumor suppressor genes after SIRT1 inhibition with pharmacological agents.

### DETAILED DESCRIPTION

The present invention relates to histone deacetylase (HDAC) inhibitors to treat cancer. The present invention provides novel class III histone deacetylase inhibitors, in particular SIRT1 inhibitors, in particular embodiments in combination with one or more agents, to reverse the silencing of hypermethylated genes, for example in cancer. The present invention is based on the finding that SIRT1 localizes to promoters of several aberrantly silenced tumor suppressor genes (TSGs) in which CpG islands are densely hypermethylated, but not to these same promoters in cell lines in which the promoters are not hypermethylated and the genes are expressed.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used in the specification and claims, the singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules.

As used herein, the term “comprising” is intended to mean that the compositions and methods include the recited elements, but do not exclude other elements. “Consisting essentially of”, when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used herein, the phrase “in combination with” is intended to refer to all forms of administration that provide a HDAC inhibitor together with a second agent, where the two are administered concurrently or sequentially in any order.

The phrase “methylation silenced genes” as used herein is meant to refer to genes that comprise a sufficient level of methylation of, e.g. CpG motifs, such that gene expression does not occur. Methylation can be, in certain preferred examples, of CpG motifs within the transcriptional regulatory region. In certain embodiments, methylation silenced genes consist of genes that are heritably repressed because of methylation of CpG islands within the transcriptional regulatory region

As used herein, “histone deacetylase” refers to a class of enzymes that selectively deacetylates the ε-amino groups of lysine located near the amino termini of core histone proteins. Mammalian HDACs have been classified into three classes: class I, II and III. HDAC inhibitors block or reduce the deacetylase activity of the HDAC enzymes.

As used herein, the term “histone deacetylase inhibitor” is meant to refer to a substance that is capable of inhibiting the histone deacetylase activity of an enzyme having histone deacetylase activity.

As used herein, the term “agent” as used herein is meant to refer to a polypeptide, polynucleotide, or fragment, or analog thereof, a small molecule, or other biologically active molecule.

As used herein, the term “hypermethylation” refers to the presence of methylated alleles in one or more nucleic acids. Any method that is sufficient to detect hypermethylation, e.g. a method that can detect methylation of nucleotides at levels as low as 0.1%, is a suitable for use in the methods of the invention. A number of different methods can be used to detect hypermethylation. In certain embodiments, hypermethylation is detected using methylation specific polymerase chain reaction (MSP).

As used herein, “epigenetic silencing” refers to a change in DNA sequence or gene expression by a process or processes that do not change the DNA coding sequence itself, but result in inhibition of gene expression. In an exemplary embodiment, methylation, for example promoter methylation, is a means of epigenetic silencing.

As used herein, the term “promoter” or “promoter region” refers to a minimal sequence sufficient to direct transcription or to render promoter-dependent gene expression that is controllable for cell-type specific, tissue-specific, or is inducible by external signals or agents. Promoters may be located in the 5′ or 3′ regions of the gene. Promoter regions, in whole or in part, of a number of nucleic acids can be examined for sites of CpG-island methylation.

As used herein, the term “proliferative disorder” refers to an abnormal growth of cells. A cell proliferative disorder as described herein may be a neoplasm. A cell proliferative disorder may also be selected from, in certain embodiments, myelofibrosis, or proliferative diabetic retinopathy. Any proliferative disorder that can be treated with a SIRT1 inhibitor is suitable for treatment by the invention as described.

As used herein, the term “neoplasm” or “neoplasia” refers to inappropriately high levels of cell division, inappropriately low levels of apoptosis, or both. A neoplasm creates an unstructured mass (a tumor), which can be either benign or malignant. For example, cancer is a neoplasia. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

As used herein, a “tumor suppressor gene” (TSG) is a gene whose product (e.g., encoded protein) is involved in negatively regulating a cancer-related process (e.g., initiation or progression or metastasis of a cancer, or inappropriate precancerous cell proliferation or survival). TSGs may encode proteins involved in, for example, negatively regulating cell growth or division, contributing to DNA repair, promoting apoptosis, inhibiting DNA replication or transcription of genes involved in growth promotion, and so forth. Examples of tumor suppressor genes include Rb, p53, INK4a, p53, APC, MLH1, MSH2, or MSH6, WTI, BRCA1, BRCA2, NF1, NF1, VHL, E-cadherin, SRFP1, SRFP2, GATA4, GATA5, cellular retinol binding protein-1.

A candidate tumor suppressor gene (candidate TSG) is a gene whose product may be (e.g., is suspected of being) involved in negatively regulating a cancer-related process (e.g., initiation or progression or metastasis of a cancer, or inappropriate precancerous cell proliferation or survival). Such a gene may be identified on the basis of hypermethylation of its promoter region. For example, if a promoter region of a gene is hypermethylated in a cancer cell, this gene is considered to be a candidate TSG. As another example, if expression of a gene is reduced in a tumor cell, the promoter can be examined for hypermethylation; if the promoter is hypermethylated, the gene is considered to be a candidate TSG and can be tested to determine if it is in fact a TSG (e.g., by determining if forced expression of the gene in the tumor cell affects growth or survival properties of the tumor cell).

As used herein, the term “subject” is meant to include vertebrates, preferably a mammal. Mammals include, but are not limited to, humans.

As used herein, the term “tumor” is intended to include an abnormal mass or growth of cells or tissue. A tumor can be benign or malignant.

### Sirtuins

Sirtuins are members of the Silent Information Regulator (SIR) family of genes. Sirtuins are proteins that include a SIR2 domain as defined as amino acids sequences that are scored as hits in the Pfam family “SIR2”—PF02146. This family is referenced in the INTERPRO database as INTERPRO description (entry IPR003000). To identify the presence of a “SIR2” domain in a protein sequence, and make the determination that a polypeptide or protein of interest has a particular profile, the amino acid sequence of the protein can be searched against the Pfam database of HMMs (e.g., the Pfam database, release 9) using the default parameters (http://www.sanger.ac.uk/Software/Pfam/HMM_search). The SIR2 domain is indexed in Pfam as PF02146 and in INTERPRO as INTERPRO description (entry IPR003000). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in “The Pfam Protein Families Database” Bateman A, Birney E, Cerruti L, Durbin R, Etwiller L, Eddy S R, Griffiths-Jones S, Howe K L, Marshall M, Sonnhammer E L (2002) Nucleic Acids Research 30(1):276-280 and Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314.

The proteins encoded by members of the SIR2 gene family may show high sequence conservation in a 250 amino acid core domain. A well-characterized gene in this family is S. cerevisiae SIR2, which is involved in silencing HM loci that contain information specifying yeast mating type, telomere position effects and cell aging (Guarente, 1999; Kaeberlein et al., 1999; Shore, 2000). The yeast Sir2 protein belongs to a family of histone deacetylases (reviewed in Guarente, 2000; Shore, 2000). The Sir2 protein is a deacetylase, which can use NAD as a cofactor (Imai et al., 2000; Moazed, 2001; Smith et al., 2000; Tanner et al., 2000; Tanny and Moazed, 2001). Unlike other deacetylases, many of which are involved in gene silencing, Sir2 is relatively insensitive to histone deacetylase inhibitors like trichostatin A (TSA) (Imai et al., 2000; Landry et al., 2000a; Smith et al., 2000). Mammalian Sir2 homologs, such as SIRT1, have NAD-dependent deacetylase activity (Imai et al., 2000; Smith et al., 2000).

The sirtuin protein family comprises members with protein deacetylase and ADP-ribosyltranferase activity. Sirtuin deacetylases are also referred to as class III deacetylases, being distinct from class I and II enzymes in that their activity depends on NAD+ (oxidized nicotinamide adenine nucleotide) and is not sensitive to the broad deacetylase inhibitor trichostatin A (TSA) (Denu J M. The Sir 2 family of protein deacetylases. Curr Opin Chem Biol 9: 431-440, 2005). Seven sirtuins have been described in humans, named SIRT1-7. SIRT1, the best characterized among them, is a nuclear deacetylase whose substrates include proteins primarily but not exclusively involved in transcriptional regulation, thus influencing diverse aspects of organismal physiology such as differentiation, cell survival, and metabolism.

Exemplary mammalian sirtuins include SIRT1, SIRT2, and SIRT3, e.g., human SIRT1, SIRT2, and SIRT3. A compound described herein may inhibit one or more activities of a mammalian sirtuin, e.g., SIRT1, SIRT2, or SIRT3, e.g., with a Ki of less than 500, 200, 100, 50, or 40 nM. For example, the compound may inhibit deacetylase activity, e.g., with respect to a natural or artificial substrate, e.g., a substrate described herein, e.g., as follows.

The SIRT1 protein is an enzyme that can remove acetyl groups attached to specific amino acids (e.g., deacetylate) in a number of different protein targets and thereby regulate gene silencing in yeast. Until the present disclosure, this had not been demonstrated in mammalian cells, and SIRT1 had not been linked to heterochromatin maintenance or heritable silencing of TSGs. Here, it is shown that SIRT1 is involved in epigenetic silencing of DNA-hypermethylated TSGs in cancer cells. Inhibition of SIRT1 by multiple approaches can lead to TSG re-expression and a block in tumor-causing networks of cell signaling that are activated by loss of the TSGs in a wide range of cancers. This finding has important ramifications for the biology of cancer in terms of what maintains abnormal gene silencing. These results demonstrate the clinical relevance of SIRT1 inhibitors, for example, in combination with a second agent such as a DNA methylation inhibitor or an HDAC I/II inhibitor as a means for restoring expression of epigenetically silenced genes, e.g., as a treatment for cancer.

Natural substrates for SIRT1 include histones, p53, and FoxO transcription factors such as FoxO1 and FoxO3. SIRT1 proteins bind to a number of other proteins, referred to as “SIRT1 binding partners.” For example, SIRT1 binds to p53 and plays a role in the p53 pathway, e.g., K370, K371, K372, K381, and/or K382 of p53 or a peptide that include one or more of these lysines. For example, the peptide can be between 5 and 15 amino acids in length. SIRT1 proteins can also deacetylate histones. For example, SIRT1 can deacetylate lysines 9 or 14 of histone H3 or small peptides that include one or more of these lysines. Histone deacetylation alters local chromatin structure and consequently can regulate the transcription of a gene in that vicinity. Many of the SIRT1 binding partners are transcription factors, e.g., proteins that recognize specific DNA sites. For example, SirT1 deacetylates and down-regulates forkhead proteins (i.e., FoxO proteins). Interaction between SIRT1 and SIRT1 binding partners can deliver SIRT1 to specific regions of a genome and can result in a local manifestation of substrates, e.g., histones and transcription factors localized to the specific region.

Natural substrates for SIRT2 include tubulin, e.g., alpha-tubulin. See, e.g., North et al. Mol. Cell. 2003 February; 11(2):437-44. Exemplary substrates include a peptide that includes lysine 40 of alpha-tubulin.

Still other exemplary sirtuin substrates include cytochrome c and acetylated peptides thereof.

Sirtuins are described in detail, e.g., in US Pub. App. No. 2006-0074124.

Exemplary compounds described herein may inhibit activity of SIRT1 by at least 10, 20, 25, 30, 50, 80, or 90%, with respect to a natural or artificial substrate described herein. For example, the compounds may have a Ki of less than 500, 200, 100, or 50 nM.

The terms “SIRT1 protein” and “SIRT1 polypeptide” are used interchangeably herein and refer a polypeptide that is at least 25% identical to the 250 amino acid conserved SIRT1 catalytic domain, amino acid residues 258 to 451 of SEQ ID NO: 2. SEQ ID NO: 2 depicts the amino acid sequence of human SIRT1. In preferred embodiments, a SIRT1 polypeptide can be at least 30, 40, 50, 60, 70, 80, 85, 90, 95, 99% homologous to SEQ ID NO: 2 or to the amino acid sequence between amino acid residues 258 and 451 of SEQ ID NO: 2. In other embodiments, the SIRT1 polypeptide can be a fragment, e.g., a fragment of SIRT1 capable of one or more of: deacetylating a substrate in the presence of NAD and/or a NAD analog and capable of binding a target protein, e.g., a transcription factor. Such functions can be evaluated, e.g., by the methods described herein. In other embodiments, the SIRT1 polypeptide can be a “full length” SIRT1 polypeptide. The term “full length” as used herein refers to a polypeptide that has at least the length of a naturally occurring SIRT1 polypeptide (or other protein described herein). A “full length” SIRT1 polypeptide or a fragment thereof can also include other sequences, e.g., a purification tag, or other attached compounds, e.g., an attached fluorophore, or cofactor. The term “SIRT1 polypeptides” can also include sequences or variants that include one or more substitutions, e.g., between one and ten substitutions, with respect to a naturally occurring Sir2 family member. A “SIRT1 activity” refers to one or more activity of SIRT1, e.g., deacetylation of a substrate (e.g., an amino acid, a peptide, or a protein), e.g., transcription factors (e.g., p53) or histone proteins, (e.g., in the presence of a cofactor such as NAD and/or an NAD analog) and binding to a target, e.g., a target protein, e.g., a transcription factor.

The GenBank accession number for the full-length human SIRT1 cDNA and its amino acids sequence, shown in SEQ ID NO: 1 and SEQ ID NO: 2 below, is NM_{—}012238:

| SEQ ID NO: 1: | |
|---|---|
| 1 | gtcgagcggg agcagaggag gcgagggagg agggccagag aggcagttgg aagatggcgg |
| 61 | acgaggcggc cctcgccctt cagcccggcg gctccccctc ggcggcgggg gccgacaggg |
| 121 | aggccgcgtc gtcccccgcc ggggagccgc tccgcaagag gccgcggaga gatggtcccg |
| 181 | gcctcgagcg gagcccgggc gagcccggtg gggcggcccc agagcgtgag gtgccggcgg |
| 241 | cggccagggg ctgcccgggt gcggcggcgg cggcgctgtg gcgggaggcg gaggcagagg |
| 301 | cggcggcggc aggcggggag caagaggccc aggcgactgc ggcggctggg gaaggagaca |
| 361 | atgggccggg cctgcagggc ccatctcggg agccaccgct ggccgacaac ttgtacgacg |
| 421 | aagacgacga cgacgagggc gaggaggagg aagaggcggc ggcggcggcg attgggtacc |
| 481 | gagataacct tctgttcggt gatgaaatta tcactaatgg ttttcattcc tgtgaaagtg |
| 541 | atgaggagga tagagcctca catgcaagct ctagtgactg gactccaagg ccacggatag |
| 601 | gtccatatac ttttgttcag caacatctta tgattggcac agatcctcga acaattctta |
| 661 | aagatttatt gccggaaaca atacctccac ctgagttgga tgatatgaca ctgtggcaga |
| 721 | ttgttattaa tatcctttca gaaccaccaa aaaggaaaaa aagaaaagat attaatacaa |
| 781 | ttgaagatgc tgtgaaatta ctgcaagagt gcaaaaaaat tatagttcta actggagctg |
| 841 | gggtgtctgt ttcatgtgga atacctgact tcaggtcaag ggatggtatt tatgctcgcc |
| 901 | ttgctgtaga cttcccagat cttccagatc ctcaagcgat gtttgatatt gaatatttca |
| 961 | gaaaagatcc aagaccattc ttcaagtttg caaaggaaat atatcctgga caattccagc |
| 1021 | catctctctg tcacaaattc atagccttgt cagataagga aggaaaacta cttcgcaact |
| 1081 | atacccagaa catagacacg ctggaacagg ttgcgggaat ccaaaggata attcagtgtc |
| 1141 | atggttcctt tgcaacagca tcttgcctga tttgtaaata caaagttgac tgtgaagctg |
| 1201 | tacgaggaga tatttttaat caggtagttc ctcgatgtcc taggtgccca gctgatgaac |
| 1261 | cgcttgctat catgaaacca gagattgtgt tttttggtga aaatttacca gaacagtttc |
| 1321 | atagagccat gaagtatgac aaagatgaag ttgacctcct cattgttatt gggtcttccc |
| 1381 | tcaaagtaag accagtagca ctaattccaa gttccatacc ccatgaagtg cctcagatat |
| 1441 | taattaatag agaacctttg cctcatctgc attttgatgt agagcttctt ggagactgtg |
| 1501 | atgtcataat taatgaattg tgtcataggt taggtggtga atatgccaaa ctttgctgta |
| 1561 | accctgtaaa gctttcagaa attactgaaa aacctccacg aacacaaaaa gaattggctt |
| 1621 | atttgtcaga gttgccaccc acacctcttc atgtttcaga agactcaagt tcaccagaaa |
| 1681 | gaacttcacc accagattct tcagtgattg tcacactttt agaccaagca gctaagagta |
| 1741 | atgatgattt agatgtgtct gaatcaaaag gttgtatgga agaaaaacca caggaagtac |
| 1801 | aaacttctag gaatgttgaa agtattgctg aacagatgga aaatccggat ttgaagaatg |
| 1861 | ttggttctag tactggggag aaaaatgaaa gaacttcagt ggctggaaca gtgagaaaat |
| 1921 | gctggcctaa tagagtggca aaggagcaga ttagtaggcg gcttgatggt aatcagtatc |
| 1981 | tgtttttgcc accaaatcgt tacattttcc atggcgctga ggtatattca gactctgaag |
| 2041 | atgacgtctt atcctctagt tcttgtggca gtaacagtga tagtgggaca tgccagagtc |
| 2101 | caagtttaga agaacccatg gaggatgaaa gtgaaattga agaattctac aatggcttag |
| 2161 | aagatgagcc tgatgttcca gagagagctg gaggagctgg atttgggact gatggagatg |
| 2221 | atcaagaggc aattaatgaa gctatatctg tgaaacagga agtaacagac atgaactatc |
| 2281 | catcaaacaa atcatagtgt aataattgtg caggtacagg aattgttcca ccagcattag |
| 2341 | gaactttagc atgtcaaaat gaatgtttac ttgtgaactc gatagagcaa ggaaaccaga |
| 2401 | aaggtgtaat atttataggt tggtaaaata gattgttttt catggataat ttttaacttc |
| 2461 | attatttctg tacttgtaca aactcaacac taactttttt ttttttaaaa aaaaaaaggt |
| 2521 | actaagtatc ttcaatcagc tgttggtcaa gactaacttt cttttaaagg ttcatttgta |
| 2581 | tgataaattc atatgtgtat atataatttt ttttgttttg tctagtgagt ttcaacattt |
| 2641 | ttaaagtttt caaaaagcca tcggaatgtt aaattaatgt aaagggacag ctaatctaga |
| 2701 | ccaaagaatg gtattttcac ttttctttgt aacattgaat ggtttgaagt actcaaaatc |
| 2761 | tgttacgcta aacttttgat tctttaacac aattattttt aaacactggc attttccaaa |
| 2821 | actgtggcag ctaacttttt aaaatctcaa atgacatgca gtgtgagtag aaggaagtca |
| 2881 | acaatatgtg gggagagcac tcggttgtct ttacttttaa aagtaatact tggtgctaag |
| 2941 | aatttcagga ttattgtatt tacgttcaaa tgaagatggc ttttgtactt cctgtggaca |
| 3001 | tgtagtaatg tctatattgg ctcataaaac taacctgaaa aacaaataaa tgctttggaa |
| 3061 | atgtttcagt tgctttagaa acattagtgc ctgcctggat ccccttagtt ttgaaatatt |
| 3121 | tgccattgtt gtttaaatac ctatcactgt ggtagagctt gcattgatct tttccacaag |
| 3181 | tattaaactg ccaaaatgtg aatatgcaaa gcctttctga atctataata atggtacttc |
| 3241 | tactggggag agtgtaatat tttggactgc tgttttccat taatgaggag agcaacaggc |
| 3301 | ccctgattat acagttccaa agtaataaga tgttaattgt aattcagcca gaaagtacat |
| 3361 | gtctcccatt gggaggattt ggtgttaaat accaaactgc tagccctagt attatggaga |
| 3421 | tgaacatgat gatgtaactt gtaatagcag aatagttaat gaatgaaact agttcttata |
| 3481 | atttatcttt atttaaaagc ttagcctgcc ttaaaactag agatcaactt tctcagctgc |
| 3541 | aaaagcttct agtctttcaa gaagttcata ctttatgaaa ttgcacagta agcatttatt |
| 3601 | tttcagacca tttttgaaca tcactcctaa attaataaag tattcctctg ttgctttagt |
| 3661 | atttattaca ataaaaaggg tttgaaatat agctgttctt tatgcataaa acacccagct |
| 3721 | aggaccatta ctgccagaga aaaaaatcgt attgaatggc catttcccta cttataagat |
| 3781 | gtctcaatct gaatttattt ggctacacta aagaatgcag tatatttagt tttccatttg |
| 3841 | catgatgttt gtgtgctata gatgatattt taaattgaaa agtttgtttt aaattatttt |
| 3901 | tacagtgaag actgttttca gctcttttta tattgtacat agtcttttat gtaatttact |
| 3961 | ggcatatgtt ttgtagactg tttaatgact ggatatcttc cttcaacttt tgaaatacaa |
| 4021 | aaccagtgtt ttttacttgt acactgtttt aaagtctatt aaaattgtca tttgactttt |
| 4081 | ttctgttaaa aaaaaaaaaa aaaaaaa |

SEQ ID NO: 2
MADEAALALQPGGSPSAAGADREAASSPAGEPLRKRPRRDGPGLERSPGEPGGAAPEREVPAAARGCPG
AAAAALWREAEAEAAAAGGEQEAQATAAAGEGDNGPGLQGPSREPPLADNLYDEDDDDEGEEEEEAAAA
AIGYRDNLLFGDEIITNGFHSCESDEEDRASHASSSDWTPRPRIGPYTFVQQHLMIGTDPRTILKDLLP
ETIPPPELDDMTLWQIVINILSEPPKRKKRKDINTIEDAVKLLQECKKIIVLTGAGVSVSCGIPDFRSR
DGIYARLAVDFPDLPDPQAMFDIEYFRKDPRPFFKFAKEIYPGQFQPSLCHKFIALSDKEGKLLRNYTQ
NIDTLEQVAGIQRIIQCHGSFATASCLICKYKVDCEAVRGDIFNQVVPRCPRCPADEPLAIMKPEIVFF
GENLPEQFHRAMKYDKDEVDLLIVIGSSLKVRPVALIPSSIPHEVPQILINREPLPHLHFDVELLGDCD
VIINELCHRLGGEYAKLCCNPVKLSEITEKPPRTQKELAYLSELPPTPLHVSEDSSSPERTSPPDSSVI
VTLLDQAAKSNDDLDVSESKGCMEEKPQEVQTSRNVESIAEQMENPDLKNVGSSTGEKNERTSVAGTVR
KCWPNRVAKEQISRRLDGNQYLFLPPNRYIFHGAEVYSDSEDDVLSSSSCGSNSDSGTCQSPSLEEPME
DESEIEEFYNGLEDEPDVPERAGGAGFGTDGDDQEAINEAISVKQEVTDMNYPSNKS

### Methylation

The propensity for cancer to arise and progress is influenced not only by gene mutations (genetic abnormalities), but also by defects in gene expression programs that are inherited from one dividing cell to another. This change in the inheritance of gene expression patterns not associated with changes in the primary DNA sequence is referred to as an epigenetic abnormality. In virtually every form of cancer, tumor suppressor genes (TSGs) and candidate TSGs are epigenetically altered such that the ability of these genes to become activated and lead to production of the corresponding proteins is lost or decreased. This so-called gene “silencing” is often linked with abnormal accumulation of methyl groups to DNA (e.g., DNA methylation or hypermethylation) in a region of the gene that controls its expression, for example, the promoter region of the gene. It can also be associated with HDAC activity (e.g., misregulated or increased HDAC activity).

DNA methylases transfer methyl groups from the universal methyl donor S-adenosyl methionine to specific sites on the DNA. Several biological functions have been attributed to the methylated bases in DNA. The most established biological function for methylated DNA is the protection of DNA from digestion by cognate restriction enzymes. The restriction modification phenomenon has, so far, been observed only in bacteria. Mammalian cells, however, possess a different methylase that exclusively methylates cytosine residues that are 5′ neighbors of guanine (CpG). This modification of cytosine residues has important regulatory effects on gene expression, especially when involving CpG rich areas, known as CpG islands, located in the promoter regions of many genes.

Methylation has been shown by several lines of evidence to play a role in gene activity, cell differentiation, tumorigenesis, X-chromosome inactivation, genomic imprinting and other major biological processes (Razin, A., H., and Riggs, R. D. eds. in DNA Methylation Biochemistry and Biological Significance, Springer-Verlag, New York, 1984). In eukaryotic cells, methylation of cytosine residues that are immediately 5′ to a guanosine occurs predominantly in CG poor regions (Bird, A., Nature, 321:209, 1986). In contrast, CpG islands remain unmethylated in normal cells, except during X-chromosome inactivation and parental specific imprinting (Li, et al., Nature, 366:362, 1993) where methylation of 5′ regulatory regions can lead to transcriptional repression. De novo methylation of the Rb gene has been demonstrated in a small fraction of retinoblastomas (Sakai, et al., Am. J. Hum. Genet., 48:880, 1991), and recently, a more detailed analysis of the VHL gene showed aberrant methylation in a subset of sporadic renal cell carcinomas (Herman, et al., Proc. Natl. Acad. Sci., U.S.A., 91:9700, 1994). Expression of a tumor suppressor gene can also be abolished by de novo DNA methylation of a normally unmethylated CpG island (Issa, et al., Nature Genet., 7:536, 1994; Herman, et al., supra; Merlo, et al., Nature Med., 1:686, 1995; Herman, et al., Cancer Res., 56:722, 1996; Graff, et al., Cancer Res., 55:5195, 1995; Herman, et al., Cancer Res., 55:4525, 1995).

In higher order eukaryotes DNA is methylated only at cytosines located 5′ to guanosine in the CpG dinucleotide. This modification has important regulatory effects on gene expression, especially when involving CpG rich areas, known as CpG islands, located in the promoter regions of many genes. While almost all gene-associated islands are protected from methylation on autosomal chromosomes, extensive methylation of CpG islands has been associated with transcriptional inactivation of selected imprinted genes and genes on the inactive X-chromosome of females. Aberrant methylation of normally unmethylated CpG islands has been described as a frequent event in immortalized and transformed cells, and has been associated with transcriptional inactivation of defined tumor suppressor genes in human cancers.

Hypermethylation can be detected using two-stage, or “nested” PCR, for example as described in U.S. Pat. No. 7,214,485, incorporated by reference in its entirety herein. For example, two-stage, or “nested” polymerase chain reaction method is disclosed for detecting methylated DNA sequences at sufficiently high levels of sensitivity to permit cancer screening in biological fluid samples, such as sputum, obtained non-invasively.

A method for assessment of the methylation status of any group of CpG sites within a CpG island, independent of the use of methylation-sensitive restriction enzymes, is described in U.S. Pat. No. 6,017,704 incorporated by reference in its entirety herein.

“Multiplex methylation-specific PCR” is a unique version of methylation-specific PCR. Methylation-specific PCR is described in U.S. Pat. Nos. 5,786,146; 6,200,756; 6,017,704 and 6,265,171, each of which is incorporated herein by reference in its entirety.

### Methods

The invention features in certain aspects methods of activating genes that are silenced by methylation in a subject comprising administering a histone deacetylase (HDAC) inhibitor. In one embodiment, gene activation comprises increased gene expression. By “activating genes that are silenced by methylation” is meant that gene activity, e.g. gene expression is restored by administration of the HDAC inhibitor. In certain preferred embodiments of the invention, activating genes that are silenced by methylation results in an increase, for example a 10%, 20%, 30%, 40%, 50%, 55%, 60%, 65%, 70%, 75%, 80% 85% 90%, 95% or more increase in the expression of gene product; however does not affect the methylation status of the gene itself, e.g. does not result in a change in the amount of methylation (e.g. decrease or increase) of the gene.

The genes that are silenced by methylation are methylated in the promoter region.

The methylation can be hypermethylation. As used herein, the term “hypermethylation” refers to the presence of methylated alleles in one or more nucleic acids. Any method that is sufficient to detect hypermethylation, e.g. a method that can detect methylation of nucleotides at levels as low as 0.1%, is a suitable for use in the methods of the invention. A number of different methods can be used to detect hypermethylation. In certain embodiments, hypermethylation is detected using methylation specific polymerase chain reaction (MSP).

In certain aspects, the invention features methods of activating genes that are silenced by methylation in a subject comprising administering a histone deacetylase (HDAC) inhibitor in combination with one or more agents. In certain embodiments, gene activation comprises increased gene expression.

In other certain aspects, the invention features methods of treating a proliferative disease or disorder comprising administering a histone deacetylase (HDAC) inhibitor in combination with one or more agents. In certain examples, the HDAC inhibitor is a class III HDAC inhibitor. As described in more detail herein the class III HDAC inhibitor can be, in certain examples, a SIRT1 inhibitor.

As described in more detail herein, in certain preferred embodiments, at least one of the one or more agents is an inhibitor of epigenetic silencing.

Thus, the invention also features methods of treating a proliferative disease or disorder comprising administering a SIRT1 inhibitor in combination with one or more agents, wherein at least one of the one or more agents is an inhibitor of epigenetic silencing.

### Diseases and Disorders Treated

The methods of the invention can be used to treat a proliferative disease or disorder. A proliferative disease or disorder characterized by unwanted cell growth. The treatments can in certain embodiments be intended to cure the proliferative disease or disorder. In other embodiments, the rearmaments are intended to provide relief from the symptoms of the proliferative disease or disorder and to prevent or arrest the development of the proliferative disease or disorder in an individual at risk from developing the proliferative disease or disorder or an individual having symptoms indicating the development of the proliferative disease or disorder in that individual.

In certain embodiments, the proliferative disease or disorder is selected from, but not limited to, a neoplasia, myelofibrosis and proliferative diabetic retinopathy. It is to be understood that proliferative diseases that can be treated by the methods of the invention are not limited to those described herein, but rather can be any proliferative disease or disorder that is responsive to treatment with a SIRT1 inhibitor or combination treatment as described herein.

Nonetheless, in certain preferred embodiments, an exemplary disease to be treated by the SIRT1 combination therapy of the invention is a neoplasia.

As used herein, the term “neoplasm” or “neoplasia” refers to inappropriately high levels of cell division, inappropriately low levels of apoptosis, or both. A neoplasm creates an unstructured mass (a tumor), which can be either benign or malignant. For example, cancer is a neoplasia. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

The compounds of the invention can be used in the treatment of cancer. The cancer can be selected from the group consisting of breast, ovarian, liver, lung, and prostate. As used herein, the terms “cancer”, “hyperproliferative”, “malignant”, and “neoplastic” are used interchangeably, and refer to those cells in an abnormal state or condition characterized by rapid proliferation or neoplasm or decreased apoptosis. The terms include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth.

The common medical meaning of the term “neoplasia” refers to “new cell growth” that results as a loss of responsiveness to normal growth controls, e.g., to neoplastic cell growth. A “hyperplasia” refers to cells undergoing an abnormally high rate of growth. However, as used herein, the terms neoplasia and hyperplasia can be used interchangeably, as their context will reveal, referring generally to cells experiencing abnormal cell growth rates. Neoplasias and hyperplasias include “tumors,” which may be benign, premalignant, or malignant.

Examples of cancerous disorders include, but are not limited to, solid tumors, soft tissue tumors, and metastatic lesions. Examples of solid tumors include malignancies, e.g., sarcomas, adenocarcinomas, and carcinomas, of the various organ systems, such as those affecting lung, breast, lymphoid, gastrointestinal (e.g., colon), and genitourinary tract (e.g., renal, urothelial cells), pharynx, prostate, ovary as well as adenocarcinomas which include malignancies such as most colon cancers, rectal cancer, renal-cell carcinoma, liver cancer, non-small cell carcinoma of the lung, cancer of the small intestine and so forth. Metastatic lesions of the aforementioned cancers can also be treated or prevented using a compound described herein.

The subject method can be useful in treating cancers of the various organ systems, such as those affecting lung, breast, lymphoid, gastrointestinal (e.g., colon), and genitourinary tract, prostate, ovary, pharynx, as well as adenocarcinomas which include malignancies such as most colon cancers, renal-cell carcinoma, prostate cancer and/or testicular tumors, non-small cell carcinoma of the lung, cancer of the small intestine and cancer of the esophagus. Exemplary solid tumors that can be treated include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

The term “carcinoma” is recognized by those skilled in the art and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is recognized by those skilled in the art and refers to malignant tumors of mesenchymal derivation.

The subject method can also be used to inhibit the proliferation of hyperplastic/neoplastic cells of hematopoietic origin, e.g., arising from myeloid, lymphoid or erythroid lineages, or precursor cells thereof. For instance, the invention contemplates the treatment of various myeloid disorders including, but not limited to, acute promyeloid leukemia (APML), acute myelogenous leukemia (AML) and chronic myelogenous leukemia (CML) (reviewed in Vaickus, L. (1991) Crit. Rev. in Oncol./Hemotol. 11:267-97). Lymphoid malignancies which may be treated by the subject method include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to, non-Hodgkin's lymphoma and variants thereof, peripheral T-cell lymphomas, adult T-cell leukemia/lymphoma (ATL), cutaneous T-cell lymphoma (CTCL), large granular lymphocytic leukemia (LGF) and Hodgkin's disease.

The compositions and methods described herein can also be used to treat pre-cancerous conditions, such as pre-leukemic syndrome myelodysplasia, benign masses of cells, erythroplasia, leukoplakia, lymphomatoid granulomatosis, lymphomatoid papulosis, preleukemia, uterine cervical dysplasia, xeroderma pigmentosum.

The compositions and methods described herein can also be used to treat a cell (e.g., in a subject, e.g., a subject that is suffering from a disorder) in which gene expression of a tumor suppressor gene or candidate tumor suppressor gene has been epigenetically decreased or silenced. The change in the inheritance of gene expression patterns not associated with changes in the primary DNA sequence is referred to as an epigenetic abnormality. In virtually every form of cancer, tumor suppressor genes (TSGs) and candidate TSGs are epigenetically altered such that the ability of these genes to become activated and lead to production of the corresponding proteins is lost or decreased. Epigenetic silencing can decrease expression of the TSG (or candidate TSG) by about 10%, about 20%, about 30%, about 40%, about 50%, about 60%, about 70%, about 80%, about 90%, about 99%, or about 100%, as compared to the expression in a normal cell. Administration of the compounds described herein can increase the expression of such epigenetically altered genes. The tumor suppressor genes can be selected from the group consisting of, but not limited to: secreted frizzled related proteins, p53, E-cadherin, mismatch repair genes, and CRBP-1.

### Therapeutics

### SIRT1 Inhibitors

The invention is based on the finding that inhibition of SIRT1 results in increased gene expression. In particular, the invention is based on the finding that inhibition of SIRT1 results in increased expression of hypermethylated, silenced TSGs.

The invention features methods of activating genes that are silenced by methylation in a subject comprising administering a histone deacetylase (HDAC) inhibitor. The invention also features methods of treating a proliferative disease or disorder, comprising administering a histone deacetylase (HDAC) inhibitor in combination with one or more agents. The HDAC inhibitor can be administered in combination with one or more agents. Preferably, the HDAC inhibitor is a class III HDAC inhibitor, more preferably in certain examples a SIRT1 inhibitor.

Non-limiting examples of negative regulators of SIRT1 include: pharmacologic inhibitors (e.g., small molecule inhibitors), DNA, RNA, RNA interfering agents, PNA, small organic molecules, natural products, proteins, antibodies, a peptides and peptidomimetics.

### Pharmacologic Inhibitors

Any number of small molecule inhibitors can be used to inhibit SIRT1 activity.

For example, one class of preferred compounds are sirtuin inhibitors, including but not limited to the sirtuin inhibitors disclosed in Grozinger et al., J. Biol. Chem. 42:38837-43 (2001), which is hereby incorporated by reference in its entirety. Preferred sirtuin inhibitors include the compounds A3, sirtinol, and M15 described therein. In one embodiment, sirtinol is a particularly preferred inhibitor.

Exemplary SIRT1 inhibitors include nicotinamide (NAM), suranim; NF023 (a G-protein antagonist); NF279 (a purinergic receptor antagonist); Trolox (6-hydroxy-2,5,7,8,tetramethylchroman-2-carboxylic acid); (−)-epigallocatechin (hydroxy on sites 3,5,7,3′,4′, 5′); (−)-epigallocatechin (hydroxy on sites 3,5,7,3′,4′, 5′); (−)-epigallocatechin gallate (Hydroxy sites 5,7,3′,4′,5′ and gallate ester on 3); cyanidin choloride (3,5,7,3′,4′-pentahydroxyflavylium chloride); delphinidin chloride (3,5,7,3′,4′,5′-hexahydroxyflavylium chloride); myricetin (cannabiscetin; 3,5,7,3′,4′,5′-hexahydroxyflavone); 3,7,3′,4′,5′-pentahydroxyflavone; and gossypetin (3,5,7,8,3′,4′-hexahydroxyflavone), all of which are further described in Howitz et al. (2003) Nature 425:191. Other inhibitors, such as sirtinol and splitomicin, are described in Grozinger et al. (2001) J. Biol. Chem. 276:38837, Dedalov et al. (2001) PNAS 98:15113 and Hirao et al. (2003) J. Biol. Chem. 278:52773. Analogs and derivatives of these compounds can also be used. Other inhibitors include Trichostatin A.

In certain embodiments, the natural products guttiferone G (1) and hyperforin (2) as well as the synthetic aristoforin (3) are used as inhibitors of human SIRT1. Hyperforin is one of the principal constituents identified in St John's wort. Hyperforin is a prenylated phloroglucinol. The structure of hyperforin is shown below:

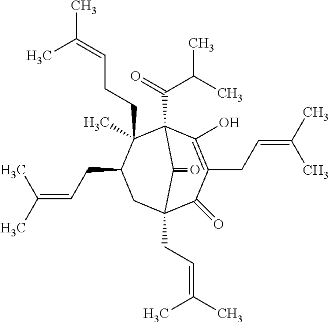

Guttiferone is a prenylated benzophenone. Guttiferone A is found in both Garcinia livingstonei T. Anders. (Gereau and Lovett 2678), originally collected in the Mufindi District of Iringa Region of Tanzania in December of 1988, and Symphonia globulifera L.f., originally collected in the Ndakan Gorilla Study Area of the Central African Republic in March 1988 (Fay 8278). Both species are members of the Clusiaceae. The structure of guttiferone is shown below:

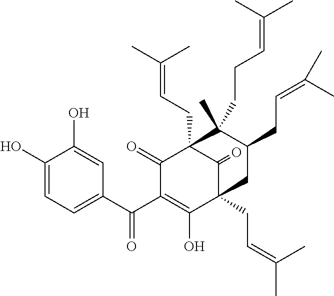

In other certain preferred embodiments, the SIRT1 inhibitors are tetrahydrocarbazole compounds. Nayagam et al., (SIRT1 modulating compounds from high-throughput screening as anti-inflammatory and insulin-sensitizing agents, J. Biomol. Screen. 2006, 11, 959-967), incorporated by reference in its entirety herein, describe tetrahydrocarbazole compounds.

US Published application No. 20060111435, incorporated by reference in its entirety herein, lists a number of sirtuin-inhibitory compounds, for example:

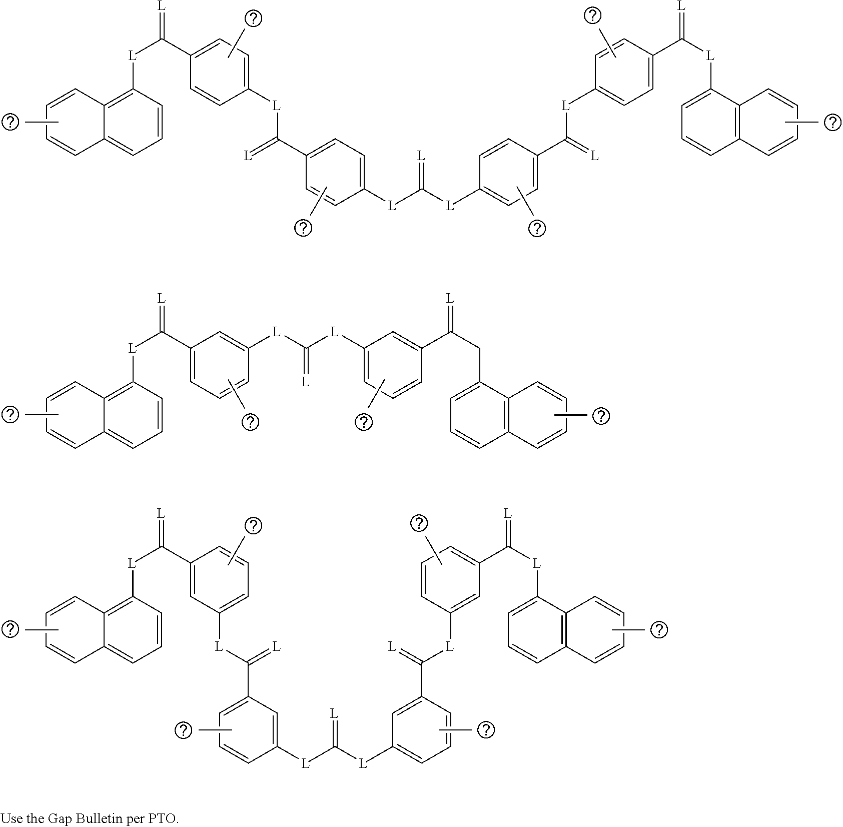

wherein, independently for each occurrence, L represents O, NR, or S; R represents H, alkyl, aryl, aralkyl, or heteroaralkyl; R′ represents H, halogen, NO.sub.2, SR, SO.sub.3, OR, NR.sub.2, alkyl, aryl, or carboxy; a represents an integer from 1 to 7 inclusively; and b represents an integer from 1 to 4 inclusively.

Inhibitory compounds may also be oxidized forms of the compounds. An oxidized form of chlortetracyclin may be an activator.

Also included are pharmaceutically acceptable addition salts and complexes of the sirtuin inhibitory compounds described herein. In cases wherein the compounds may have one or more chiral centers, unless specified, the compounds contemplated herein may be a single stereoisomer or racemic mixtures of stereoisomers.

In cases in which the sirtuin inhibitory compounds have unsaturated carbon-carbon double bonds, both the cis (Z) and trans (E) isomers are contemplated herein. In cases wherein the compounds may exist in tautomeric forms, such as keto-enol tautomers, such as each tautomeric form is contemplated as being included within the methods presented herein, whether existing in equilibrium or locked in one form by appropriate substitution with R′. The meaning of any substituent at any one occurrence is independent of its meaning, or any other substituents meaning, at any other occurrence.

Also included in the methods presented herein are prodrugs of the sirtuin inhibitory compounds described herein. Prodrugs are considered to be any covalently bonded carriers that release the active parent drug in vivo. Metabolites, such as in vivo degradation products, of the compounds described herein are also included.

US Published application 20070043050, incorporated by reference in its entirety herein, describes sirtuin-modulating compounds. Sirtuin-modulating compounds can be as below, or a salt thereof:

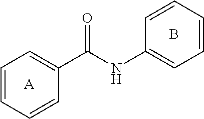

Ring A is optionally substituted, fused to another ring or both; and Ring B is substituted with at least one carboxy, substituted or unsubstituted arylcarboxamine, substituted or unsubstituted aralkylcarboxamine, substituted or unsubstituted heteroaryl group, substituted or unsubstituted heterocyclylcarbonylethenyl, or polycyclic aryl group or is fused to an aryl ring and is optionally substituted by one or more additional groups.

Optionally, the sirtuin-modulating compound can be of the formula below, or a salt thereof:

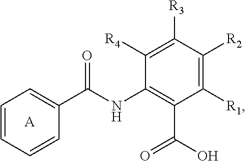

Ring A is optionally substituted; R.sub.1, R.sub.2, R.sub.3 and R.sub.4 are independently selected from the group consisting of —H, halogen, —OR.sub.5, —CN, —CO.sub.2R.sub.5, —OCOR.sub.5, —OCO.sub.2R.sub.5, —C(O)NR.sub.5R.sub.6, —OC(O)NR.sub.5R.sub.6, —C(O)R.sub.5, —COR.sub.5, —SR.sub.5, —OSO.sub.3H, —S(O).sub.nR.sub.5, —S(O).sub.nOR.sub.5, —S(O).sub.nNR.sub.5R.sub.6, —NR.sub.5R.sub.6, —NR.sub.5C(O)OR.sub.6, —NR.sub.5C(O)R.sub.6 and —NO.sub.2; R.sub.5 and R.sub.6 are independently —H, a substituted or unsubstituted alkyl group, a substituted or unsubstituted aryl group or a substituted or unsubstituted heterocyclic group; and n is 1 or 2.

Any one or more of the compounds listed in US Published application 20070043050, US Published application 20070037827, US Published application 20070037865, US Published application 20060276393, and US Published application 20060229265 all of which are incorporated by reference in their entireties herein, are suitable for use in the invention.

Other SIRT1 inhibitors that can be used in practicing the invention have a general formula (I) and contain a substituted five or six membered ring core containing one or two, respectively, oxygen, nitrogen, or sulfur atoms as a constituent

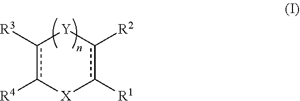

atom of the ring, e.g., X and Y in formula (I) below.

Any ring carbon atom can be substituted. For example, R¹, R², R³, and R⁴ may include without limitation substituted or unsubstituted alkyl, cycloalkyl, alkenyl, alkynyl, heterocyclyl, heterocycloalkenyl, cycloalkenyl, aryl, heteroaryl, etc. The five or six membered ring core may be saturated, i.e. containing no double bonds, or partially or fully saturated, i.e. one or two double bonds respectively. When n=0, “X” may be oxygen, sulfur, or nitrogen, e.g., NR⁷. The substituent R⁷ can be without limitation hydrogen, alkyl, e.g., C1, C2, C3, C4 alkyl, SO₂(aryl), acyl, or the ring nitrogen may form part of a carbamate, or urea group. When n=1, X can be NR⁷, O, or S; and Y can be NR^{7′}, O or S. X and Y can be any combination of heteroatoms, e,g., N, N, N, O, N, S, etc.

A preferred subset of compounds of formula (I) includes those having one, or preferably, two rings that are fused to the five or six membered ring core, e.g., R¹ and R², together with the carbons to which they are attached, and/or R³ and R⁴, together with the carbons to which they are attached, can form, e.g., C₅-C₁₀ cycloalkyl (e.g., C5, C6, or C7), C₅-C₁₀ heterocyclyl (e.g., C5, C6, or C7), C₅-C₁₀ cycloalkenyl (e.g., C5, C6, or C7), C₅-C₁₀ heterocycloalkenyl (e.g., C5, C6, or C7), C₆-C₁₀ aryl (e.g., C6, C8 or C10), or C₆-C₁₀ heteroaryl (e.g., C5 or C6). Fused ring combinations may include without limitation one or more of the following:

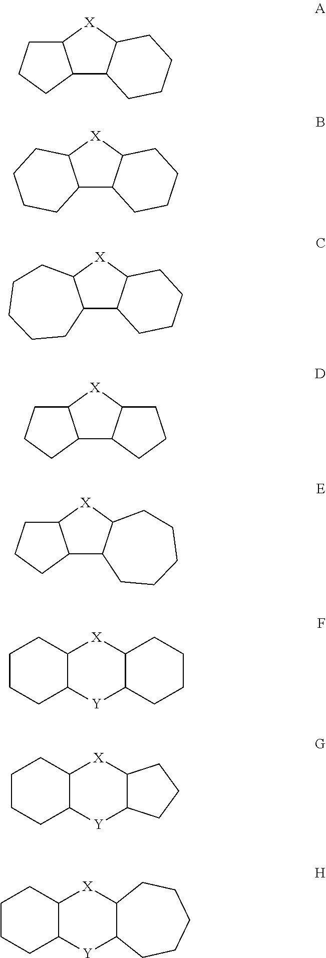

Preferred combinations include B, e.g. having C₆ aryl and C₆ cycloalkenyl (B1), and C, e.g. having C₆ aryl and C₇ cycloalkenyl (C1):

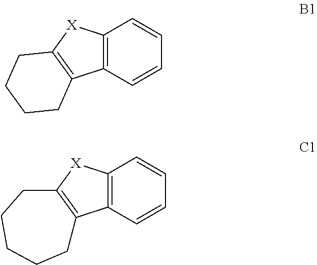

Each of these fused ring systems may be optionally substituted with substituents, which may include without limitation halo, hydroxy, C₁-C₁₀ alkyl (C1,C2,C3,C4,C5,C6,C7,C8,C9,C10), C₁-C₆ haloalkyl (C1,C2,C3,C4,C5,C6,), C₁-C₁₀ alkoxy (C1,C2,C3,C4,C5,C6,C7,C8,C9,C10), C₁-C₆ haloalkoxy (C1,C2,C3,C4,C5,C6,), C₆-C₁₀ aryl (C6,C7,C8,C9,C10), C₅-C₁₀ heteroaryl (C5,C6,C7,C8,C9,C10), C₇-C₁₂ aralkyl (C7,C8,C9,C10,C11,C12), C₇-C₁₂ heteroaralkyl (C7,C8,C9,C10,C11,C12), C₃-C₈ heterocyclyl (C3,C4,C5,C6,C7,C8), C₂-C₁₂ alkenyl (C2,C3,C4,C5,C6,C7,C8,C9,C10,C11,C12), C₂-C₁₂ alkynyl (C2,C3,C4,C5,C6,C7,C8,C9,C10,C11,C12), C₅-C₁₀ cycloalkenyl (C5,C6,C7,C8,C9,C10), C₅-C₁₀ heterocycloalkenyl (C5,C6,C7,C8,C9,C10), carboxy, carboxylate, cyano, nitro, amino, C₁-C₆ alkyl amino (C1,C2,C3,C4,C5,C6,), C₁-C₆ dialkyl amino (C1,C2,C3,C4,C5,C6,), mercapto, SO₃H, sulfate, S(O)NH₂, S(O)₂NH₂, phosphate, C₁-C₄ alkylenedioxy (C1,C2,C3,C4), oxo, acyl, aminocarbonyl, C₁-C₆ alkyl aminocarbonyl (C1,C2,C3,C4,C5,C6,), C₁-C₆ dialkyl aminocarbonyl (C1,C2,C3,C4,C5,C6,), C₁-C₁₀ alkoxycarbonyl (C1,C2,C3,C4,C5,C6,C7,C8,C9,C10), C₁-C₁₀ thioalkoxycarbonyl (C1,C2,C3,C4,C5,C6,C7,C8,C9,C10), hydrazinocarbonyl, C₁-C₆ alkyl hydrazinocarbonyl (C1,C2,C3,C4,C5,C6,), C₁-C₆ dialkyl hydrazinocarbonyl (C1,C2,C3,C4,C5,C6,), hydroxyaminocarbonyl, etc. Preferred substituents include halo (e.g., fluoro, chloro, bromo), C₁-C₁₀ alkyl (e.g., C1, C2, C3, C4, C5, C6, C7, C8, C9, C10), C₁-C₆ haloalkyl (e.g., C1, C2, C3, C4, C5, C6, e.g., CF₃), C₁-C₆ haloalkoxyl (e.g., C1, C2, C3, C4, C5, C6, e.g., OCF₃), or aminocarbonyl. The substitution pattern on the two fused rings may be selected as desired, e.g., one ring may be substituted and the other is not, or both rings may be substituted with 1-5 substitutents (1,2,3,4,5 substitutents). The number of substituents on each ring may be the same or different. Preferred substitution patterns are shown below:

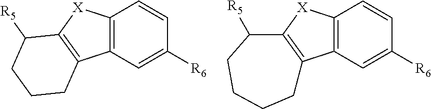

In certain embodiments, when n is 0 and X is NR⁷, the nitrogen substituent R⁷ can form a cyclic structure with one of the fused rings containing, e.g., 4-6 carbons, 1-3 nitrogens, 0-2 oxygens and 0-2 sulfurs. This cyclic structure may optionally be substituted with oxo or C₁-C₆ alkyl.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

Exemplary SIRT1 inhibitors include those depicted in Table 1 below*:

TABLE 1
Exemplary SIRT1 inhibitors

| Compound | | Ave. SIRT1 p53-382 |
|---|---|---|
| number | Chemical name | IC50 (μM) |
| 1 | 7-Chloro-1,2,3,4-tetrahydro-cyclopenta[b]indole-3- | A |
| | carboxylic acid amide | |
| 2 | 2,3,4,9-Tetrahydro-1H-b-carboline-3-carboxylic acid amide | C |
| 3 | 6-Bromo-2,3,4,9-tetrahydro-1H-carbazole-2-carboxylic acid | B |
| | amide | |
| 4 | 6-Methyl-2,3,4,9-tetrahydro-1H-carbazole-1-carboxylic acid | A |
| | amide | |
| 5 | 2,3,4,9-Tetrahydro-1H-carbazole-1-carboxylic acid amide | B |
| 6 | 2-Chloro-5,6,7,8,9,10-hexahydro-cyclohepta[b]indole-6- | A |
| | carboxylic acid amide | |
| 7 | 6-Chloro-2,3,4,9-tetrahydro-1H-carbazole-1-carboxylic acid | C |
| | hydroxyamide | |
| 8 | 6-Chloro-2,3,4,9-tetrahydro-1H-carbazole-1-carboxylic acid | A |
| | amide | |
| 9 | 6-Chloro-2,3,4,9-tetrahydro-1H-carbazole-2-carboxylic acid | C |
| | amide | |
| 10 | 1,2,3,4-Tetrahydro-cyclopenta[b]indole-3-carboxylic acid | B |
| | amide | |
| 11 | 6-Chloro-2,3,4,9-tetrahydro-1H-carbazole-1-carboxylic acid | B |
| | (5-chloro-pyridin-2-yl)-amide | |
| 12 | 1,6-Dimethyl-2,3,4,9-tetrahydro-1H-carbazole-1-carboxylic | C |
| | acid amide | |
| 13 | 6-Trifluoromethoxy-2,3,4,9-tetrahydro-1H-carbazole-2- | C |
| | carboxylic acid amide | |
| 14 | 6-Chloro-2,3,4,9-tetrahydro-1H-carbazole-1-carboxylic acid | D |
| | diethylamide | |
| 15 | 6-Chloro-2,3,4,9-tetrahydro-1H-carbazole-1-carboxylic acid | D |
| | carbamoylmethyl-amide | |
| 16 | 8-Carbamoyl-6,7,8,9-tetrahydro-5H-carbazole-1-carboxylic | D |
| | acid | |
| 17 | 6-Methyl-2,3,4,9-tetrahydro-1H-carbazole-1-carboxylic acid | D |
| 18 | 8-Carbamoyl-2,3,4,9-tetrahydro-1H-carbazole-1-carboxylic | D |
| | acid ethyl ester | |
| 19 | [(6-Chloro-2,3,4,9-tetrahydro-1H-carbazole-1-carbonyl)- | D |
| | amino]-acetic acid ethyl ester | |
| 20 | 9-Benzyl-2,3,4,9-tetrahydro-1H-carbazole-1-carboxylic acid | D |
| | amide | |
| 21 | 6-Chloro-2,3,4,9-tetrahydro-1H-carbazole-1-carboxylic acid | D |
| | methyl ester | |
| 22 | 6-Chloro-2,3,4,9-tetrahydro-1H-carbazole-1-carboxylic acid | D |
| 23 | C-(6-Methyl-2,3,4,9-tetrahydro-1H-carbazol-1-yl)- | D |
| | methylamine | |
| 24 | 6,9-Dimethyl-2,3,4,9-tetrahydro-1H-carbazole-1-carboxylic | D |
| | acid amide | |
| 25 | 7-Methyl-1,2,3,4-tetrahydro-cyclopenta[b]indole-3- | D |
| | carboxylic acid amide | |
| 26 | 6-Chloro-2,3,4,9-tetrahydro-1H-carbazole-1-carboxylic acid | D |
| | ethylamide | |
| 27 | 2-(1-Benzyl-3-methylsulfanyl-1H-indol-2-yl)-N-p-tolyl- | D |
| | acetamide | |
| 28 | N-Benzyl-2-(1-methyl-3-phenylsulfanyl-1H-indol-2-yl)- | D |
| | acetamide | |
| 29 | N-(4-Chloro-phenyl)-2-(1-methyl-3-phenylsulfanyl-1H-indol- | D |
| | 2-yl)-acetamide | |
| 30 | N-(3-Hydroxy-propyl)-2-(1-methyl-3-phenylsulfanyl-1H- | D |
| | indol-2-yl)-acetamide | |
| 31 | 2-(1-Benzyl-3-phenylsulfanyl-1H-indol-2-yl)-N-(3-hydroxy- | D |
| | propyl)-acetamide | |
| 32 | 2-(1-Benzyl-3-methylsulfanyl-1H-indol-2-yl)-N-(4-methoxy- | D |
| | phenyl)-acetamide | |
| 33 | 2-(1-Benzyl-1H-indol-2-yl)-N-(4-methoxy-phenyl)-acetamide | D |
| 34 | 2-(1-Methyl-3-methylsulfanyl-1H-indol-2-yl)-N-p-tolyl- | D |
| | acetamide | |
| 35 | 2-(1-Benzyl-3-methylsulfanyl-1H-indol-2-yl)-N-(2-chloro- | D |
| | phenyl)-acetamide | |
| 36 | 2-(1,5-Dimethyl-3-methylsulfanyl-1H-indol-2-yl)-N-(2- | D |
| | hydroxy-ethyl)-acetamide | |
| 37 | (6-Chloro-2,3,4,9-tetrahydro-1H-carbazol-1-yl)-[4-(furan-2- | D |
| | carbonyl)-piperazin-1-yl]-methanone | |
| 38 | 2-(1-Benzyl-1H-indol-2-yl)-N-(2-chloro-phenyl)-acetamide | D |
| 39 | 6-Chloro-2,3,4,9-tetrahydro-1H-carbazole-1-carboxylic acid | D |
| | ethyl ester | |
| 40 | 6-Chloro-9-methyl-2,3,4,9-tetrahydro-1H-carbazole-4- | D |
| | carboxylic acid ethyl ester | |
| 41 | 5,7-Dichloro-2,3,4,9-tetrahydro-1H-carbazole-1-carboxylic | D |
| | acid ethyl ester | |
| 42 | 7-Chloro-2,3,4,9-tetrahydro-1H-carbazole-1-carboxylic acid | D |
| | ethyl ester | |
| 43 | 5,7-Dichloro-2,3,4,9-tetrahydro-1H-carbazole-1-carboxylic | D |
| | acid | |
| 44 | 6-Chloro-9-methyl-2,3,4,9-tetrahydro-1H-carbazole-4- | D |
| | carboxylic acid | |
| 45 | 6-Chloro-9-methyl-2,3,4,9-tetrahydro-1H-carbazole-4- | D |
| | carboxylic acid amide | |
| 46 | 6-Morpholin-4-yl-2,3,4,9-tetrahydro-1H-carbazole-1- | D |
| | carboxylic acid ethyl ester | |
| 47 | 6-Morpholin-4-yl-2,3,4,9-tetrahydro-1H-carbazole-1- | D |
| | carboxylic acid amide | |
| 48 | 6-Bromo-2,3,4,9-tetrahydro-1H-carbazole-1-carboxylic acid | D |
| | ethyl ester | |
| 49 | 6-Fluoro-2,3,4,9-tetrahydro-1H-carbazole-1-carboxylic acid | D |
| | ethyl ester | |
| 50 | 3-Carbamoyl-1,3,4,9-tetrahydro-b-carboline-2-carboxylic | D |
| | acid tert-butyl ester | |
| 51 | 6-Chloro-2,3,4,9-tetrahydro-1H-carbazole-1-carboxylic acid | D |
| | (1-phenyl-ethyl)-amide | |
| 52 | 7,8-Difluoro-2,3,4,9-tetrahydro-1H-carbazole-1-carboxylic | D |
| | acid amide | |
| 53 | 6-bromo-2,3,4,9-tetrahydro-1H-carbazole-1-carboxylic acid | D |
| 54 | 6-hydroxy-2,3,4,9-tetrahydro-1H-carbazole-1-carboxylic | C |
| | acid | |
| 55 | 6-bromo-2,3,4,9-tetrahydro-1H-carbazole-2-carboxamide | B |
| 56 | 6-chloro-2,3,4,9-tetrahydro-1H-pyrido[3,4-b]indole-1- | C |
| | carboxamide | |
| 57 | 6-bromo-2,3,4,9-tetrahydro-1H-pyrido[3,4-b]indole-1- | D |
| | carboxamide | |
| 58 | 2-acetyl-6-chloro-2,3,4,9-tetrahydro-1H-pyrido[3,4-b]indole- | C |
| | 1-carboxamide | |

*Compounds having activity designated with an A have an IC₅₀ of less than 1.0 μM. Compounds having activity designated with a B have an IC₅₀ between 1.0 μM and 10.0 μM. Compounds having activity designated with a C have an IC₅₀ greater than 10.0 μM. Compounds designated with a D were not tested in this assay.

Compounds that can be useful in practicing this invention can be identified through both in vitro (cell and non-cell based) and in vivo methods. A description of these methods is described in the Examples.

Exemplary compounds are also described, e.g., in US Pub. App. US 2006-0074124.

### Synthesis of Compounds

The compounds described herein can be obtained from commercial sources (e.g., Asinex, Moscow, Russia; Bionet, Camelford, England; ChemDiv, SanDiego, Calif.; Comgenex, Budapest, Hungary; Enamine, Kiev, Ukraine; IF Lab, Ukraine; Interbioscreen, Moscow, Russia; Maybridge, Tintagel, UK; Specs, The Netherlands; Timtec, Newark, Del.; Vitas-M Lab, Moscow, Russia) or synthesized by conventional methods as shown below using commercially available starting materials and reagents. For example, exemplary compound 4 can be synthesized as shown in Scheme 1 below.

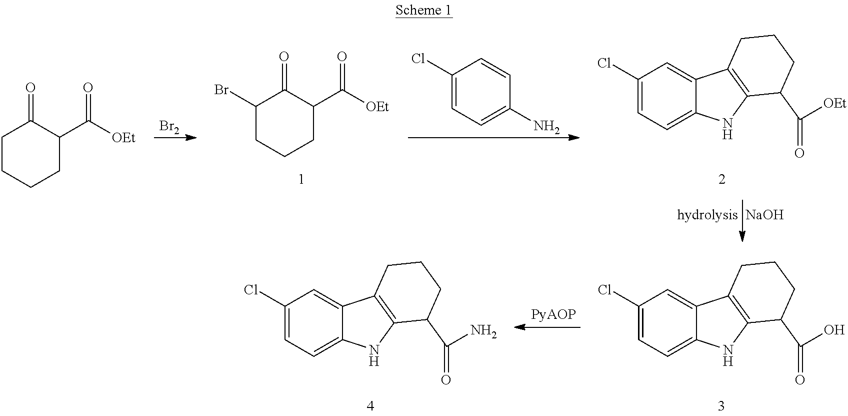

Brominated β-keto ester 1 can be condensed with 4-chloroaniline followed by cyclization can afford indole 2. Ester saponification can afford acid 3. Finally amination with PyAOP can yield the amide 4. Other methods are known in the art, see, e.g., U.S. Pat. No. 3,859,304, U.S. Pat. No. 3,769,298, J. Am. Chem. Soc. 1974, 74, 5495. The synthesis above can be extended to other anilines, e.g., 3,5-dichloroaniline,

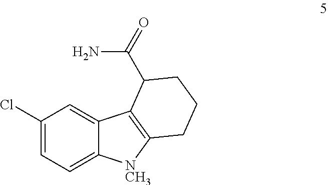

3-chloroaniline, and 4-bromoaniline. Regioisomeric products, e.g., 5, may be obtained using N-substituted anilines, e.g., 4-chloro-N-methylaniline.

The compounds described herein can be separated from a reaction mixture and further purified by a method such as column chromatography, high-pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e.g., carbon-carbon bonds) or substituents that can restrict bond rotation, e.g. restriction resulting from the presence of a ring or double bond. Accordingly, all cis/trans and E/Z isomers are expressly included in the present invention. The compounds of this invention may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

Techniques useful for the separation of isomers, e.g., stereoisomers are within skill of the art and are described in Eliel, E. L.; Wilen, S. H.; Mander, L. N. Stereochemistry of Organic Compounds, Wiley Interscience, NY, 1994. For example compound 3 or 4 can be resolved to a high enantiomeric excess (e.g., 60%, 70%, 80%, 85%, 90%, 95%, 99% or greater) via formation of diasteromeric salts, e.g. with a chiral base, e.g., (+) or (−) α-methylbenzylamine, or via high performance liquid chromatography using a chiral column. In some embodiments, the crude product 4, is purified directly on a chiral column to provide enantiomerically enriched compound.

For purposes of illustration, enantiomers of compound 4 are shown below.

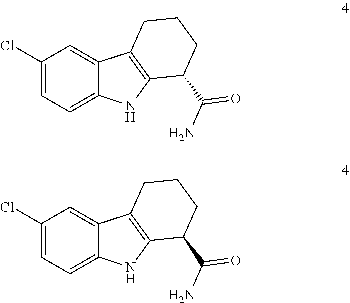

In some instances, the compounds disclosed herein are administered where one isomer (e.g., the R isomer or S isomer) is present in high enantiomeric excess. In general, the isomer of compound 4 having a negative optical rotation, e.g., −14.1 (c=0.33, DCM) or [α]_{D}²⁵−41.18° (c 0.960, CH₃OH) has greater activity against the SIRT1 enzyme than the enantiomer that has a positive optical rotation of +32.8 (c=0.38, DCM) or [α]_{D}²⁵+22.72° (c 0.910, CH₃OH). Accordingly, in some instances, it is beneficial to administer to a subject a compound 4 having a high enantiomeric excess of the isomer having a negative optical rotation to treat a disease.

While the enantiomers of compound 4 provide one example of a stereoisomer, other stereoisomers are also envisioned, for example as depicted in compounds 6 and 7 below.

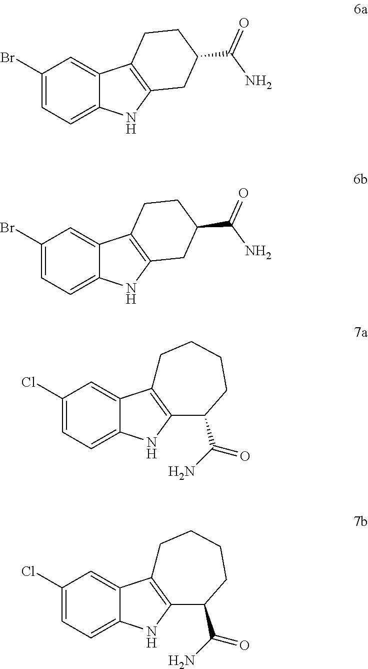

As with the compound of formula 4, in some instances it is beneficial to administer to a subject an isomer of compounds 6 or 7 that has a greater affinity for SIRT1 than its enantiomer. For example, in some instances, it is beneficial to administer a compound 7, enriched with the (−) optical rotamer, wherein the amide (or other substituent) has the same configuration as the negative isomer of compound 4.

In some instances, it is beneficial to administer a compound having the one of the following structures where the stereochemical structure of the amide (or other substituent) corresponds to the amide in compound 4 having a negative optical rotation.

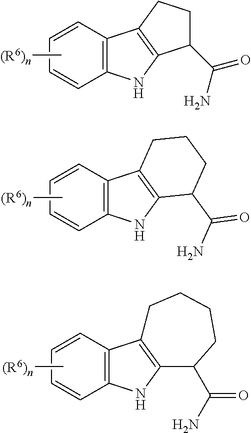

(n is an integer from 0 to 4.)

The compounds of this invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active compounds.

The compounds of this invention may be modified by appending appropriate functionalities to enhance selected biological properties, e.g., targeting to a particular tissue. Such modifications are known in the art and include those which increase biological penetration into a given biological compartment (e.g., blood, lymphatic system, central nervous system), increase oral availability, increase solubility to allow administration by injection, alter metabolism and alter rate of excretion.

In an alternate embodiment, the compounds described herein may be used as platforms or scaffolds that may be utilized in combinatorial chemistry techniques for preparation of derivatives and/or chemical libraries of compounds. Such derivatives and libraries of compounds have biological activity and are useful for identifying and designing compounds possessing a particular activity. Combinatorial techniques suitable for utilizing the compounds described herein are known in the art as exemplified by Obrecht, D. and Villalgrodo, J. M., Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, A. W., Curr. Opin. Chem. Bio., (1997) 1, 60. Thus, one embodiment relates to a method of using the compounds described herein for generating derivatives or chemical libraries comprising: 1) providing a body comprising a plurality of wells; 2) providing one or more compounds identified by methods described herein in each well; 3) providing an additional one or more chemicals in each well; 4) isolating the resulting one or more products from each well. An alternate embodiment relates to a method of using the compounds described herein for generating derivatives or chemical libraries comprising: 1) providing one or more compounds described herein attached to a solid support; 2) treating the one or more compounds identified by methods described herein attached to a solid support with one or more additional chemicals; 3) isolating the resulting one or more products from the solid support. In the methods described above, “tags” or identifier or labeling moieties may be attached to and/or detached from the compounds described herein or their derivatives, to facilitate tracking, identification or isolation of the desired products or their intermediates. Such moieties are known in the art. The chemicals used in the aforementioned methods may include, for example, solvents, reagents, catalysts, protecting group and deprotecting group reagents and the like. Examples of such chemicals are those that appear in the various synthetic and protecting group chemistry texts and treatises referenced herein.

Other examples of SIRT1 inhibitors that can be used in the compositions and methods described herein include those disclosed in U.S. Patent Application No. 2005/0250794, the contents of which are hereby incorporated by reference in its entirety.

### Oligonucleotide Agents

As used herein, an “oligonucleotide agent” refers to a single stranded oligomer or polymer of ribonucleic acid (RNA) or deoxyribonucleic acid (DNA) or both or modifications thereof, which is antisense with respect to its target. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

Oligonucleotide agents include both nucleic acid targeting (NAT) oligonucleotide agents and protein-targeting (PT) oligonucleotide agents. NAT and PT oligonucleotide agents refer to single stranded oligomers or polymers of ribonucleic acid (RNA) or deoxyribonucleic acid (DNA) or both or modifications thereof. NATs designed to bind to specific RNA or DNA targets have substantial complementarity, e.g., at least 70, 80, 90, or 100% complementary, with at least 10, 20, or 30 or more bases of a target nucleic acid, and include antisense RNAs, microRNAs, antagomirs and other non-duplex structures which can modulate expression. The NAT oligonucleotide agents can target any nucleic acid, e.g., a miRNA, a pre-miRNA, a pre-mRNA, an mRNA, or a DNA. These NAT oligonucleotide agents may or may not bind via Watson-Crick complementarity to their targets. PT oligonucleotide agents bind to protein targets, preferably by virtue of three-dimensional interactions, and modulate protein activity. They include decoy RNAs, aptamers, and the like.

### Single Stranded Ribonucleic Acid

Oligonucleotide agents include microRNAs (miRNAs). MicroRNAs are small noncoding RNA molecules that are capable of causing post-transcriptional silencing of specific genes in cells such as by the inhibition of translation or through degradation of the targeted mRNA. An miRNA can be completely complementary or can have a region of noncomplementarity with a target nucleic acid, consequently resulting in a “bulge” at the region of non-complementarity. The region of noncomplementarity (the bulge) can be flanked by regions of sufficient complementarity, preferably complete complementarity to allow duplex formation. Preferably, the regions of complementarity are at least 8 to 10 nucleotides long (e.g., 8, 9, or 10 nucleotides long). A miRNA can inhibit gene expression by repressing translation, such as when the microRNA is not completely complementary to the target nucleic acid, or by causing target RNA degradation, which is believed to occur only when the miRNA binds its target with perfect complementarity. The invention also can include double-stranded precursors of miRNAs that may or may not form a bulge when bound to their targets.

In a preferred embodiment an oligonucleotide agent featured in the invention can target an endogenous miRNA or pre-miRNA. The oligonucleotide agent featured in the invention can include naturally occurring nucleobases, sugars, and covalent internucleoside (backbone) linkages as well as oligonucleotides having non-naturally-occurring portions that function similarly. Such modified or substituted oligonucleotides are often preferred over native forms because of desirable properties such as, for example, enhanced cellular uptake, enhanced affinity for the endogenous miRNA target, and/or increased stability in the presence of nucleases. An oligonucleotide agent designed to bind to a specific endogenous miRNA has substantial complementarity, e.g., at least 70, 80, 90, or 100% complementary, with at least 10, 20, or 25 or more bases of the target miRNA.

A miRNA or pre-miRNA can be 18-100 nucleotides in length, and more preferably from 18-80 nucleotides in length. Mature miRNAs can have a length of 19-30 nucleotides, preferably 21-25 nucleotides, particularly 21, 22, 23, 24, or 25 nucleotides. MicroRNA precursors can have a length of 70-100 nucleotides and have a hairpin conformation. MicroRNAs can be generated in vivo from pre-miRNAs by enzymes called Dicer and Drosha that specifically process long pre-miRNA into functional miRNA. The microRNAs or precursor mi-RNAs featured in the invention can be synthesized in vivo by a cell-based system or can be chemically synthesized. MicroRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism. Modifications can also increase sequence specificity, and consequently decrease off-site targeting.

An miRNA or a pre-miRNA can be constructed using chemical synthesis and/or enzymatic ligation reactions using procedures known in the art. For example, an miRNA or a pre-miRNA can be chemically synthesized using naturally occurring nucleotides or variously modified nucleotides designed to increase the biological stability of the molecules or to increase the physical stability of the duplex formed between the miRNA or a pre-miRNA and target nucleic acids, e.g., phosphorothioate derivatives and acridine substituted nucleotides can be used. Other appropriate nucleic acid modifications are described herein. Alternatively, the miRNA or pre-miRNA nucleic acid can be produced biologically using an expression vector into which a nucleic acid has been subcloned in an antisense orientation (i.e., RNA transcribed from the inserted nucleic acid will be of an antisense orientation to a target nucleic acid of interest).

Preferably, SIRT1 expression may be inhibited ex vivo by the use of any method which results in decreased transcription of the gene encoding SIRT1. The sequence of the gene encoding mouse SIRT1 is available in Genbank as genomic contig accession number NT 039495 (Mus musculus chromosome 10 genomic contig). The accession number for the gene encoding mouse SIRT1 is available in Genbank at NM._{—}019812 (Mus musculus sirtuin 1). The sequence of the gene encoding human is available in Genbank as accession number NM_{—}012238 as shown in SEQ ID NO: 1 herein.

Double-Stranded Ribonucleic Acid (dsRNA)

In one embodiment, the invention provides a double-stranded ribonucleic acid (dsRNA) molecule packaged in an association complex, such as a liposome, for inhibiting the expression of a gene in a cell or mammal, wherein the dsRNA comprises an antisense strand comprising a region of complementarity which is complementary to at least a part of an mRNA formed in the expression of the gene, and wherein the region of complementarity is less than 30 nucleotides in length, generally 19-24 nucleotides in length, and wherein said dsRNA, upon contact with a cell expressing said gene, inhibits the expression of said gene by at least 40%. The dsRNA comprises two RNA strands that are sufficiently complementary to hybridize to form a duplex structure. One strand of the dsRNA (the antisense strand) comprises a region of complementarity that is substantially complementary, and generally fully complementary, to a target sequence, derived from the sequence of an mRNA formed during the expression of a gene, the other strand (the sense strand) comprises a region which is complementary to the antisense strand, such that the two strands hybridize and form a duplex structure when combined under suitable conditions. Generally, the duplex structure is between 15 and 30, more generally between 18 and 25, yet more generally between 19 and 24, and most generally between 19 and 21 base pairs in length. Similarly, the region of complementarity to the target sequence is between 15 and 30, more generally between 18 and 25, yet more generally between 19 and 24, and most generally between 19 and 21 nucleotides in length. The dsRNA of the invention may further comprise one or more single-stranded nucleotide overhang(s). The dsRNA can be synthesized by standard methods known in the art as further discussed below, e.g., by use of an automated DNA synthesizer, such as are commercially available from, for example, Biosearch, Applied Biosystems, Inc.

The dsRNAs suitable for packaging in the association complexes described herein can include a duplex structure of between 18 and 25 basepairs (e.g., 21 base pairs). In some embodiments, the dsRNAs include at least one strand that is at least 21 nt long. In other embodiments, the dsRNAs include at least one strand that is at least 15, 16, 17, 18, 19, 20, or more contiguous nucleotides.

### RNA Interference

In one preferred embodiment, RNAi technology can be used to inhibit or downregulate the expression of SIRT1 by decreasing transcription of the gene encoding SIRT1. RNA interference or “RNAi” is a term initially coined by Fire and co-workers to describe the observation that double-stranded RNA (dsRNA) can block gene expression when it is introduced into worms (Fire et al. (1998) Nature 391, 806-811). “RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target genes. See for example U.S. patent application Ser. Nos: 20030153519A1; 20030167490A1; and U.S. Pat. Nos. 6,506,559; 6,573,099, which are herein incorporated by reference in their entirety.

Isolated RNA molecules specific to SIRT1 mRNA, which mediate RNAi, are antagonists useful in the method of the present invention. In one embodiment, the RNA interfering agents used in the methods of the invention, e.g., the siRNAs used in the methods of the invention, can to be taken up actively by cells ex vivo by their addition to the culture medium, illustrating efficient delivery of the RNA interfering agents, e.g., the siRNAs used in the methods of the invention.

An “RNA interfering agent” as used herein, is defined as any agent which interferes with or inhibits expression of a target gene or genomic sequence by RNA interference (RNAi). Such RNA interfering agents include, but are not limited to, nucleic acid molecules including RNA molecules which are homologous to the target gene or genomic sequence, or a fragment thereof, short interfering RNA (siRNA), short hairpin or small hairpin RNA (shRNA), and small molecules which interfere with or inhibit expression of a target gene by RNA interference (RNAi). The target gene of the present invention is the gene encoding SIRT1.

“Short interfering RNA” (siRNA), also referred to herein as “small interfering RNA” is defined as an agent which functions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, 5, or 6 nucleotides. The length of the overhang is independent between the two strands, i.e., the length of the overhang on one strand is not dependent on the length of the overhang on the second strand. In one embodiment, the siRNA can inhibit SIRT1 s by transcriptional silencing. Preferably the siRNA is capable of promoting RNA interference through degradation or specific post-transcriptional gene silencing (PTGS) of the target messenger RNA (mRNA).

To induce RNA interference in a cell, dsRNA may be introduced into the cell as an isolated nucleic acid fragment or via a transgene, plasmid or virus. Alternatively, siRNA may be synthesized and introduced directly into the cell. Other strategies for delivery of the RNA interfering agents, e.g., the siRNAs or shRNAs of used in the methods of the invention, may also be employed, such as, for example, delivery by a vector, e.g., a plasmid or viral vector, e.g., a lentiviral vector. Other delivery methods include delivery of the RNA interfering agents, e.g., the siRNAs or shRNAs of the invention, using a basic peptide by conjugating or mixing the RNA interfering agent with a basic peptide, e.g., a fragment of a TAT peptide, mixing with cationic lipids or formulating into particles.

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501, incorporated be reference herein).

siRNA sequences are selected on the basis of their homology to the gene it is desired to silence. Homology between two nucleotide sequences may be determined using a variety of programs including the BLAST program, of Altschul et al. (1990) J. Mol. Biol. 215: 403-10, or BestFit, which is part of the Wisconsin Package, Version 8, September 1994, (Genetics Computer Group, 575 Science Drive, Madison, Wis., USA, Wisconsin 53711). Sequence comparisons may be made using FASTA and FASTP (see Pearson & Lipman, 1988. Methods in Enzymology 183: 63-98). Parameters are preferably set, using the default matrix, as follows: Gapopen (penalty for the first residue in a gap): −16 for nucleic acid; Gapext (penalty for additional residues in a gap): −4 for nucleic acids; KTUP word length: 6 for nucleic acids.

Sequence comparison may be made over the full length of the relevant sequence, or may more preferably be over a contiguous sequence of about or 10, 15, 20, 25 or 30 bases. Preferably the degree of homology between the siRNA and the target gene is at least 75%, at least 80%, at least 85%, at least 90%, at least 95%, at least 97%, or at least 99%.

The degree of homology between the siRNA or dsRNA and the gene to be silenced will preferably be sufficient that the siRNA or dsRNA will hybridize to the nucleic acid of the gene sequence under stringent hybridization conditions.

Typical hybridization conditions use 4-6.times.SSPE; 5-10.times. Denhards solution, 5 g polyvinylpyrrolidone and 5 g bovine serum albumin; 100.ug-lmg/ml sonicated salmon sperm DNA; 0.1-1% sodium dodecyl sulphate; optionally 40-60% deionized formamide. Hybridization temperature will vary depending on the GC content of the nucleic acid target sequence but will typically be between 42.degree. C.-65.degree. C. Sambrook et al (2001) Molecular Cloning: A Laboratory Approach (3.sup.rd Edn, Cold Spring Harbor Laboratory Press). A common formula for calculating the stringency conditions required to achieve hybridization between nucleic acid molecules of a specified homology is: Tₘ=81.5C.+16.6 Log [Na⁺]+0.41[% G+C]−0.63 (% formamide).

The siRNA may be between 10 bp and 30 bp in length, preferably between 20 by and 25 bp. Preferably, the siRNA is 19, 20, 21 or 22 bp in length.

The siRNA sequence may be, for example, any suitable contiguous sequence of 10-30 bp from the sequence shown below:

| SEQ ID NO: 1: | |
|---|---|
| 1 | gtcgagcggg agcagaggag gcgagggagg agggccagag aggcagttgg aagatggcgg |
| 61 | acgaggcggc cctcgccctt cagcccggcg gctccccctc ggcggcgggg gccgacaggg |
| 121 | aggccgcgtc gtcccccgcc ggggagccgc tccgcaagag gccgcggaga gatggtcccg |
| 181 | gcctcgagcg gagcccgggc gagcccggtg gggcggcccc agagcgtgag gtgccggcgg |
| 241 | cggccagggg ctgcccgggt gcggcggcgg cggcgctgtg gcgggaggcg gaggcagagg |
| 301 | cggcggcggc aggcggggag caagaggccc aggcgactgc ggcggctggg gaaggagaca |
| 361 | atgggccggg cctgcagggc ccatctcggg agccaccgct ggccgacaac ttgtacgacg |
| 421 | aagacgacga cgacgagggc gaggaggagg aagaggcggc ggcggcggcg attgggtacc |
| 481 | gagataacct tctgttcggt gatgaaatta tcactaatgg ttttcattcc tgtgaaagtg |
| 541 | atgaggagga tagagcctca catgcaagct ctagtgactg gactccaagg ccacggatag |
| 601 | gtccatatac ttttgttcag caacatctta tgattggcac agatcctcga acaattctta |
| 661 | aagatttatt gccggaaaca atacctccac ctgagttgga tgatatgaca ctgtggcaga |
| 721 | ttgttattaa tatcctttca gaaccaccaa aaaggaaaaa aagaaaagat attaatacaa |
| 781 | ttgaagatgc tgtgaaatta ctgcaagagt gcaaaaaaat tatagttcta actggagctg |
| 841 | gggtgtctgt ttcatgtgga atacctgact tcaggtcaag ggatggtatt tatgctcgcc |
| 901 | ttgctgtaga cttcccagat cttccagatc ctcaagcgat gtttgatatt gaatatttca |
| 961 | gaaaagatcc aagaccattc ttcaagtttg caaaggaaat atatcctgga caattccagc |
| 1021 | catctctctg tcacaaattc atagccttgt cagataagga aggaaaacta cttcgcaact |
| 1081 | atacccagaa catagacacg ctggaacagg ttgcgggaat ccaaaggata attcagtgtc |
| 1141 | atggttcctt tgcaacagca tcttgcctga tttgtaaata caaagttgac tgtgaagctg |
| 1201 | tacgaggaga tatttttaat caggtagttc ctcgatgtcc taggtgccca gctgatgaac |
| 1261 | cgcttgctat catgaaacca gagattgtgt tttttggtga aaatttacca gaacagtttc |
| 1321 | atagagccat gaagtatgac aaagatgaag ttgacctcct cattgttatt gggtcttccc |
| 1381 | tcaaagtaag accagtagca ctaattccaa gttccatacc ccatgaagtg cctcagatat |
| 1441 | taattaatag agaacctttg cctcatctgc attttgatgt agagcttctt ggagactgtg |
| 1501 | atgtcataat taatgaattg tgtcataggt taggtggtga atatgccaaa ctttgctgta |
| 1561 | accctgtaaa gctttcagaa attactgaaa aacctccacg aacacaaaaa gaattggctt |
| 1621 | atttgtcaga gttgccaccc acacctcttc atgtttcaga agactcaagt tcaccagaaa |
| 1681 | gaacttcacc accagattct tcagtgattg tcacactttt agaccaagca gctaagagta |
| 1741 | atgatgattt agatgtgtct gaatcaaaag gttgtatgga agaaaaacca caggaagtac |
| 1801 | aaacttctag gaatgttgaa agtattgctg aacagatgga aaatccggat ttgaagaatg |
| 1861 | ttggttctag tactggggag aaaaatgaaa gaacttcagt ggctggaaca gtgagaaaat |
| 1921 | gctggcctaa tagagtggca aaggagcaga ttagtaggcg gcttgatggt aatcagtatc |
| 1981 | tgtttttgcc accaaatcgt tacattttcc atggcgctga ggtatattca gactctgaag |
| 2041 | atgacgtctt atcctctagt tcttgtggca gtaacagtga tagtgggaca tgccagagtc |
| 2101 | caagtttaga agaacccatg gaggatgaaa gtgaaattga agaattctac aatggcttag |
| 2161 | aagatgagcc tgatgttcca gagagagctg gaggagctgg atttgggact gatggagatg |
| 2221 | atcaagaggc aattaatgaa gctatatctg tgaaacagga agtaacagac atgaactatc |
| 2281 | catcaaacaa atcatagtgt aataattgtg caggtacagg aattgttcca ccagcattag |
| 2341 | gaactttagc atgtcaaaat gaatgtttac ttgtgaactc gatagagcaa ggaaaccaga |
| 2401 | aaggtgtaat atttataggt tggtaaaata gattgttttt catggataat ttttaacttc |
| 2461 | attatttctg tacttgtaca aactcaacac taactttttt ttttttaaaa aaaaaaaggt |
| 2521 | actaagtatc ttcaatcagc tgttggtcaa gactaacttt cttttaaagg ttcatttgta |
| 2581 | tgataaattc atatgtgtat atataatttt ttttgttttg tctagtgagt ttcaacattt |
| 2641 | ttaaagtttt caaaaagcca tcggaatgtt aaattaatgt aaagggacag ctaatctaga |
| 2701 | ccaaagaatg gtattttcac ttttctttgt aacattgaat ggtttgaagt actcaaaatc |
| 2761 | tgttacgcta aacttttgat tctttaacac aattattttt aaacactggc attttccaaa |
| 2821 | actgtggcag ctaacttttt aaaatctcaa atgacatgca gtgtgagtag aaggaagtca |
| 2881 | acaatatgtg gggagagcac tcggttgtct ttacttttaa aagtaatact tggtgctaag |
| 2941 | aatttcagga ttattgtatt tacgttcaaa tgaagatggc ttttgtactt cctgtggaca |
| 3001 | tgtagtaatg tctatattgg ctcataaaac taacctgaaa aacaaataaa tgctttggaa |
| 3061 | atgtttcagt tgctttagaa acattagtgc ctgcctggat ccccttagtt ttgaaatatt |
| 3121 | tgccattgtt gtttaaatac ctatcactgt ggtagagctt gcattgatct tttccacaag |
| 3181 | tattaaactg ccaaaatgtg aatatgcaaa gcctttctga atctataata atggtacttc |
| 3241 | tactggggag agtgtaatat tttggactgc tgttttccat taatgaggag agcaacaggc |
| 3301 | ccctgattat acagttccaa agtaataaga tgttaattgt aattcagcca gaaagtacat |
| 3361 | gtctcccatt gggaggattt ggtgttaaat accaaactgc tagccctagt attatggaga |
| 3421 | tgaacatgat gatgtaactt gtaatagcag aatagttaat gaatgaaact agttcttata |
| 3481 | atttatcttt atttaaaagc ttagcctgcc ttaaaactag agatcaactt tctcagctgc |
| 3541 | aaaagcttct agtctttcaa gaagttcata ctttatgaaa ttgcacagta agcatttatt |
| 3601 | tttcagacca tttttgaaca tcactcctaa attaataaag tattcctctg ttgctttagt |
| 3661 | atttattaca ataaaaaggg tttgaaatat agctgttctt tatgcataaa acacccagct |
| 3721 | aggaccatta ctgccagaga aaaaaatcgt attgaatggc catttcccta cttataagat |
| 3781 | gtctcaatct gaatttattt ggctacacta aagaatgcag tatatttagt tttccatttg |
| 3841 | catgatgttt gtgtgctata gatgatattt taaattgaaa agtttgtttt aaattatttt |
| 3901 | tacagtgaag actgttttca gctcttttta tattgtacat agtcttttat gtaatttact |
| 3961 | ggcatatgtt ttgtagactg tttaatgact ggatatcttc cttcaacttt tgaaatacaa |
| 4021 | aaccagtgtt ttttacttgt acactgtttt aaagtctatt aaaattgtca tttgactttt |
| 4081 | ttctgttaaa aaaaaaaaaa aaaaaaa |

Alternatively, longer dsRNA fragments comprising contiguous sequences from the sequences complementary to SEQ ID NO: 1 may be used, as they will be cleaved to form siRNAs within the cell. In certain preferred examples, the siRNA sequences comprise SEQ ID NO:3 (RNAi-1), SEQ ID NO: 4(RNAi-2) or SEQ ID NO:5 (RNAi-3), as shown below:

| | |
|---|---|
| SEQ ID NO: 3 (RNAi-1) | CTTGTACGACGAAGACGAC |
| SEQ ID NO: 4 (RNAi-2) | GGCCACGGATAGGTCCATA |
| SEQ ID NO: 5 (RNAi-3) | CATAGACACGCTGGAACAG |

In some embodiments, the siRNA has an overhang at one or both ends of one or more deoxythymidine bases. The overhang is not to be interpreted as part of the siRNA sequence. Where present, it serves to increase the stability of the siRNA within cells by reducing its susceptibility to degradation by nucleases.

siRNA molecules may be synthesized using standard solid or solution phase synthesis techniques which are known in the art. Linkages between nucleotides may be phosphodiester bonds or alternatives, for example, linking groups of the formula P(O)S, (thioate); P(S)S, (dithioate); P(O)NR12; P(O)R′; P(O)OR6; CO; or CONR′2 wherein R is H (or a salt) or alkyl (1-12C) and R6 is alkyl (1-9C) is joined to adjacent nucleotides through —O— or —S—.

Alternatively, siRNA molecules or longer dsRNA molecules may be made recombinantly by transcription of a nucleic acid sequence, preferably contained within a vector as described below.

Modified nucleotide bases can be used in addition to the naturally occurring bases, and may confer advantageous properties on siRNA molecules containing them.

For example, modified bases may increase the stability of the siRNA molecule, thereby reducing the amount required for silencing. The provision of modified bases may also provide siRNA molecules which are more, or less, stable than unmodified siRNA.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases may also be modified. Any modified base useful for inhibiting or interfering with the expression of a target sequence may be used. For example, halogenated bases, such as 5-bromouracil and 5-iodouracil can be incorporated. The bases may also be alkylated, for example, 7-methylguanosine can be incorporated in place of a guanosine residue. Non-natural bases that yield successful inhibition can also be incorporated. In a preferred embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

Modified nucleotides are known in the art and include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N6-methyladenine, 4-acetylcytosine, 5-(carboxyhydroxylmethyl)uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2-methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5-methoxyuracil, 2 methylthio-N6-isopentenyladenine, uracil-5-oxyacetic acid methyl ester, psueouracil, 2-thiocytosine, 5-methyl-2 thiouracil, 2-thiouracil, 4-thiouracil, 5-methyluracil, N-uracil-5-oxyacetic acid methylester, uracil 5-oxyacetic acid, queosine, 2-thiocytosine, 5-propyluracil, 5-propylcytosine, 5-ethyluracil, 5-ethylcytosine, 5-butyluracil, 5-pentyluracil, 5-pentylcytosine, and 2,6,diaminopurine, methylpsuedouracil, 1-methylguanine, 1-methylcytosine.

### Ribozymes

Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. The composition of ribozyme molecules includes one or more sequences complementary to the target gene mRNA, and includes the well known catalytic sequence responsible for mRNA cleavage disclosed, for example, in U.S. Pat. No. 5,093,246. Within the scope of this disclosure are engineered hammerhead motif ribozyme molecules that specifically and efficiently catalyze endonucleolytic cleavage of RNA sequences encoding target gene proteins. Specific ribozyme cleavage sites within any potential RNA target are initially identified by scanning the molecule of interest for ribozyme cleavage sites that include the sequences GUA, GUU, and GUC. Once identified, short RNA sequences of between 15 and 20 ribonucleotides corresponding to the region of the target gene containing the cleavage site may be evaluated for predicted structural features, such as secondary structure, that may render the oligonucleotide sequence unsuitable. The suitability of candidate sequences may also be evaluated by testing their accessibility to hybridization with complementary oligonucleotides, using ribonuclease protection assays.

The antisense, ribozyme, and/or triple helix molecules described herein may reduce or inhibit the transcription (triple helix) and/or translation (antisense, ribozyme) of mRNA produced by both normal and mutant target gene alleles.

### Aptamer-Type Oligonucleotide Agents

An oligonucleotide agent featured in the invention can be an aptamer. An aptamer binds to a non-nucleic acid ligand, such as a small organic molecule or protein, e.g., a transcription or translation factor, and subsequently modifies (e.g., inhibits) activity. An aptamer can fold into a specific structure that directs the recognition of the targeted binding site on the non-nucleic acid ligand. An aptamer can contain any of the modifications described herein.

In one embodiment, an aptamer includes a modification that improves targeting, e.g. a targeting modification described herein.

The chemical modifications described above for miRNAs and antisense RNAs, and described elsewhere herein, are also appropriate for use in decoy nucleic acids.

Exemplary shRNAis include the following sequences:

I. pSUPERretro-SIRT1-RNAi-1 (NM_{—}012238 positions 410):

Target sequence:
CTTGTACGACGAAGACGAC
Forward primer:
GATCCCCCTTGTACGACGAAGACGACTTCAAGAGAGTCGTCTTC
GTCGTACAAGTTTTTGGAAA
Reverse primer:
AGCTTTTCCAAAAACTTGTACGACGAAGACGACTCTCTTGAAGT
CGTCTTCGTCGTACAAGGGG

II. pSUPERretro-SIRT1-RNAi-2 (NM_{—}012238 positions 589):

Target sequence:
GGCCACGGATAGGTCCATAT
Forward primer:
GATCCCCGGCCACGGATAGGTCCATATTCAAGAGATATGGACCT
ATCCGTGGCCTTTTTGGAAA
Reverse primer:
AGCTTTTCCAAAAAGGCCACGGATAGGTCCATATCTCTTGAATA
TGGACCTATCCGTGGCCGGG

III. pSUPERretro-SIRT1-RNAi-3 (NM_{—}012238 positions 1091):

Target sequence:
CATAGACACGCTGGAACAG
Forward primer:
GATCCCCCATAGACACGCTGGAACAGTTCAAGAGACTGTTCCAG
CGTGTCTATGTTTTTGGAAA
Reverse primer:
AGCTTTTCCAAAAACATAGACACGCTGGAACAGTCTCTTGAACT
GTTCCAGCGTGTCTATGGGG

### Vectors

The invention also provides vectors comprising a nucleotide sequence encoding an siRNA or longer RNA or DNA sequence for production of dsRNA. The vector may be any RNA or DNA vector. The vector is preferably an expression vector, wherein the nucleotide sequence is operably linked to a promoter compatible with the cell. The vector will preferably have at least two promoters, one to direct expression of the sense strand and one to direct expression of the antisense strand of the dsRNA. Alternatively, two vectors may be used, one for the sense strand and one for the antisense strand. Alternatively the vector may encode RNAs which form stem-loop structures which are subsequently cleaved by the cell to produce dsRNA.

Where the vector is an expression vector, the sequence to be expressed will preferably be operably linked to a promoter functional in the target cells. Promoters suitable for use in various vertebrate systems are well known. For example, suitable promoters include viral promoters such as mammalian retrovirus or DNA virus promoters, e.g. MLV, CMV, RSV, SV40 IEP and adenovirus promoters and metallothionein promoter. The CMV IEP may be more preferable for human use. Strong mammalian promoters may also be suitable as well as RNA polymerase II and III promoters. Variants of such promoters retaining substantially similar transcriptional activities may also be used.

Other vehicles suitable for use in delivering nucleic acids such as siRNAs include viruses and virus-like particles (VLPs) such as HPV VLPs comprising the L1 and/or L2 HPV viral protein; or hepatitis B viral proteins. Other suitable VLPs may be derived from picornaviruses; togaviruses; rhabdoviruses; orthomyxoviruses; retroviruses; hepadnaviruses; papovaviruses; adenoviruses; herpesviruses; and pox viruses.

The RNA interfering agents, e.g., the siRNAs or shRNAs of the invention, may be introduced along with components that perform one or more of the following activities: enhance uptake of the RNA interfering agents, inhibit annealing of single strands, stabilize single strands, or otherwise facilitate delivery to the target cell and increase inhibition of the target gene, SIRT1.

### Delivery

Various agents may be used to improve the delivery of RNA, DNA or protein into the cell. Viral vectors as described above may be used to deliver nucleic acid into a cell. Where other vectors, or no vector, are used, delivery agents such as liposomes may usefully be employed. Delivery peptides such as Antennapedia of the HIV TAT peptide may be used, as may organic polymers such as a dendrimers or polylysine-transferrine-conjugates.

Liposomes can be prepared from a variety of cationic lipids, including DOTAP, DOTMA, DDAB, L-PE, and the like. Lipid carrier mixtures containing a cationic lipid, such as N-[1-(2,3-dioleyloxy)propyl]-N,N,N-triethylammonium chloride (DOTMA) also known as “lipofectin”, dimethyl dioctadecyl ammonium bromide (DDAB), 1,2-dioleoyloxy-3-(trimethylammonio) propane (DOTAP) or L-lysinyl-phosphatidylethanolamine (L-PE) and a second lipid, such as dioleoylphosphatidylethanolamine (DOPE) or cholesterol (Chol), are particularly useful for use with nucleic acids. DOTMA synthesis is described in Felgner, et al., (1987) Proc. Nat. Acad. Sciences, (USA) 84:7413-7417. DOTAP synthesis is described in Stamatatos, et al., Biochemistry, (1988) 27:3917-3925.

Liposomes are commercially available from many sources. DOTMA:DOPE lipid carriers can be purchased from, for example, BRL. DOTAP:DOPE lipid carriers can be purchased from Boehringer Mannheim. Cholesterol and DDAB are commercially available from Sigma Corporation. DOPE is commercially available from Avanti Polar Lipids. DDAB:DOPE can be purchased from Promega. Invitrogen make liposomes under the names OLIGOFECTAMINE and LIPOFECTAMINE.

To incorporate nucleic acid into liposomes, the liposome-nucleic acid complex is prepared by mixing with the nucleic acid in an appropriate nucleic acid:lipid ratio (for example 5:3) in a physiologically acceptable diluent (for example OPTI-MEM at an appropriate dilution) immediately prior to use.

Another delivery system for polynucleotides is a colloidal dispersion system. Colloidal dispersion systems include macromolecular complexes, nanocapsules, microspheres, beads, and lipid-based systems including oil-in-water emulsions, micelles, mixed micelles and liposomes. A preferred colloidal delivery system is a liposome, an artificial membrane vesicle useful as in vivo or in vitro delivery vehicles. The composition of a liposome is usually a combination of phospholipids, usually in combination with steroids, particularly cholesterol.

### Inhibitors of Epigenetic Silencing

The SIRT1 inhibitors described herein can be used in combination with a second agent, e.g., an inhibitor of epigenetic silencing, e.g., an inhibitor of epigenetic silencing, such as an agent that decreases DNA methylation (e.g., an inhibitor of DNA methylation (e.g., a DNA methyltransferase inhibitor) or an agent that promotes DNA demethylase activity) or an agent that decreases histone deacetylation (e.g., an inhibitor of type I/II HDACs (e.g., a class I and/or class II histone deacetylase inhibitor (HDI)) or an agent that promotes histone acetylation (e.g., histone acetyl transferase activity)).

An inhibitor described herein can increase the expression of a gene by at least about 10%, at least about 20%, at least about 30%, at least about 40%, at least about 50%, at least about 60%, at least about 70%, at least about 80%, at least about 90%, at least about 95%, at least about 99%, or at least about 100% as compared to the level of expression of the gene under identical conditions but in the absence of the inhibitor. The gene can be, e.g., a gene that has been epigenetically silenced, e.g., by HDAC activity and/or by DNA methylation (e.g., hypermethylation, e.g., hypermethylation of CpG islands) in the gene's promoter region. An inhibitor of a DNA methyltransferase can decrease the methyltransferase activity by at least about 10%, at least about 20%, at least about 30%, at least about 40%, at least about 50%, at least about 60%, at least about 70%, at least about 80%, at least about 90%, at least about 95%, at least about 99%, or at least about 100% as compared to the level of methyltransferase activity of the DNA methyltransferase under identical conditions but in the absence of the inhibitor. A DNA demethylating agent can decrease the amount of methylation of a gene promoter (e.g., CpG islands) by at least about 10%, at least about 20%, at least about 30%, at least about 40%, at least about 50%, at least about 60%, at least about 70%, at least about 80%, at least about 90%, at least about 95%, at least about 99%, or at least about 100% as compared to the level of methylation under identical conditions but in the absence of the demethylating agent. An HDAC inhibitor can decrease the deacetylase activity by at least about 10%, at least about 20%, at least about 30%, at least about 40%, at least about 50%, at least about 60%, at least about 70%, at least about 80%, at least about 90%, at least about 95%, at least about 99%, or at least about 100% as compared to the level of deacetylase activity of the HDAC under identical conditions but in the absence of the inhibitor.

Proteins involved in DNA methylation include DNA methyltransferases, such as mammalian DNA methyltransferases DNMT1, DNMT2, DNMT3A and DNMT3B. Non-limiting examples of agents that decrease DNA methylation (e.g., by demethylating DNA or inhibiting the action of DNA methyltransferases) include nucleoside DNA methyltransferase inhibitors and non-nucleoside DNA methyltransferase inhibitors (see, e.g., Lyko and Brown, J. National Cancer Inst. 97(20):1498-1506 (2005)). Examples of nucleoside DNA methyltransferase inhibitors include 5-deoxy-azacytidine (DAC), 5-azacytidine (5-aza-CR) (Vidaza), 5-aza-2′-deoxycytidine (5-aza-CdR; decitabine), 1-β-D-arabinofuranosyl-5-azacytosine, dihydro-5-azacytidine, zebularine, Sinefungin (e.g., INSOLUTION™ Sinefungin), 5-fluoro-2′-deoxycyticine (FdCyd). Examples of non-nucleoside DNA methyltransferase inhibitors (e.g., other than procaine) include: (−)-epigallocatechin-3-gallate (EGCG), RG108, hydralazine, procainamide, 1513-DMIa and 1513-DMIb which were isolated from the culture filtrate of Streptomyces sp. strain No. 1513, psammaplin, dominant negative forms of the DNA methyltransferases (e.g., catalytically inactive forms), oligonucleotides (e.g., including hairpin loops and specific antisense oligonucleotides (such as MG98)), siRNA inhibitors of the DNA methyltransferases, and antibodies that specifically bind to the DNA methyltransferases. Inhibitors are available, e.g., from Merck Biosciences.

An agent that decreases DNA methylation can be an agent that activates and/or promotes DNA demethylase activity or decreases DNA methyl transferase activity. The agent, for example, can act directly on the enzyme, e.g., by interacting with the enzyme in a competitive, non-competitive or an uncompetitive manner. The agent can also decrease DNA methylation by increasing the expression of a protein that either decreases DNA methylation and/or promotes DNA demethylase activity or decrease the expression of a protein that promotes DNA methylation and/or promotes DNA methyl transferase activity.

Type I mammalian HDACs include: HDAC1, HDAC2, HDAC3, HDAC8, and HDAC11. Type II mammalian HDACs include: HDAC4, HDAC5, HDAC6, HDAC7, HDAC9, and HDAC1.

A number of structural classes of negative regulators of HDACs (e.g., HDAC inhibitors) have been developed, for example, small molecular weight carboxylates (e.g., less than about 250 amu), hydroxamic acids, benzamides, epoxyketones, cyclic peptides, and hybrid molecules. (See for example, Drummond D C, Noble C O, Kirpotin D B, Guo Z, Scott G K, et al. (2005) Clinical development of histone deacetylase inhibitors as anticancer agents. Annu Rev Pharmacol Toxicol 45: 495-528, (including specific examples therein) which is hereby incorporated by reference in its entirety). Non-limiting examples of negative regulators of type I/II HDACs include: Suberoylanilide Hydroxamic Acid (SAHA (e.g., MK0683, vorinostat) and other hydroxamic acids), Suberoyl bis-hydroxamic Acid, BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid (and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (i.e., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-Cl-UCHA (i.e., 6-(3-chlorophenylureido)caproic hydroxamic acid), AOE (2-amino-8-oxo-9,10-epoxydecanoic acid), CHAP31 and CHAP 50. Other inhibitors include, for example, dominant negative forms of the HDACs (e.g., catalytically inactive forms) siRNA inhibitors of the HDACs, and antibodies that specifically bind to the HDACs. Inhibitors are available, e.g., from BIOMOL International, Fukasawa, Merck Biosciences, Novartis, Gloucester Pharmaceuticals, Aton Pharma, Titan Pharmaceuticals, Schering AG, Pharmion, MethylGene, and Sigma Aldrich.

An agent that promotes histone acetylation can be an agent that activates and/or promotes histone acetyl transferase activity, e.g., activates histone acetylase activity, e.g., that is mediated by histone acetyl transferases. Histone acetyl transferases (HATs) include: PCAF, CBP, GCN5, p300/CREB, Esa1, Hat1, proteins of the p160 family, TAFII250, and Tip60.

The SIRT1 inhibitors described herein can also be used in combination with another SIRT inhibitor, such as a general SIRT inhibitor, such as sirtinol.

### Identifying HDACIII Inhibitors

In certain aspects, the invention features methods that can be used to identify novel HDACIII inhibitors. In exemplary embodiments, a method used to identify SIRT1 inhibitors comprises administering a candidate compound to a cell with one or more genes that are silenced by methylation in vitro; and determining whether gene expression in increased in said cell; wherein increased gene expression compared to untreated cells identifies a SIRT1 inhibitor.

In the methods, the SIRT1 inhibitors that are identified, in certain embodiments, do not affect gene methylation.

### Pharmaceutical Compositions

### Combinations

In certain aspects, the invention features pharmaceutical compositions including a combination of a SIRT1 inhibitor and an inhibitor of epigenetic silencing. The SIRT1 inhibitor and the second agent (e.g., an inhibitor of epigenetic silencing, e.g., a DNA demethylating agent/inhibitor of a DNA methyltransferase or an inhibitor of type I/II HDACs) may be formulated in separate dosage forms. Alternatively, to decrease the number of dosage forms administered to a subject, each agent may be formulated together in any combination. For example, the SIRT1 inhibitor may be formulated in one dosage form and any additional agents may be formulated together or in another dosage form. The SIRT1 inhibitor can be dosed, for example, before, after or during the dosage of the additional agent.

Combinations of a SIRT1 Inhibitor with an Inhibitor of Epigenetic Silencing

The present disclosure provides, inter alia, the use of a SIRT1 inhibitor in combination with a second agent, such as an inhibitor of epigenetic silencing, such as an agent that decreases DNA methylation (e.g., an inhibitor of DNA methylation (e.g., a DNA methyltransferase inhibitor) or an agent that promotes DNA demethylase activity) or an agent that decreases histone deacetylation (e.g., an inhibitor of type I/II HDACs (e.g., a class I and/or class II histone deacetylase inhibitor) or an agent that promotes histone acetylation (e.g., histone acetyl transferase activity)). The combination can be used, for example, to increase expression of a gene (e.g., TSG or candidate TSG) and/or to decrease the amount of DNA methylation at a promoter and/or to decrease the histone deacetylase activity at a promoter. The combination of agents described herein can have additive or synergistic effects on gene expression of one or more epigenetically silenced genes, for example, by inhibition of SIRT1 or of epigenetic silencing. Preferably, the effects are synergistic (e.g., the two agents produce an effect greater than the sum of their individual effects).

A combination of inhibitors described herein can increase the expression of a gene by at least about 10%, at least about 20%, at least about 30%, at least about 40%, at least about 50%, at least about 60%, at least about 70%, at least about 80%, at least about 90%, at least about 95%, at least about 99%, or at least about 100% as compared to the level of expression of the gene under identical conditions but in the absence of the combination. The gene can be, e.g., a gene that has been epigenetically silenced, e.g., by HDAC activity and/or by DNA methylation (e.g., hypermethylation, e.g., hypermethylation of CpG islands) in the gene's promoter region.

A combination of inhibitors described herein can increase the expression of a gene by at least about 10%, at least about 20%, at least about 30%, at least about 40%, at least about 50%, at least about 60%, at least about 70%, at least about 80%, at least about 90%, at least about 95%, at least about 99%, or at least about 100% as compared to the level of expression of the gene under identical conditions but in the absence of the combination and in the presence of an agent that decreases DNA methylation. The gene can be, e.g., a gene that has been epigenetically silenced, e.g., by HDAC activity and/or by DNA methylation (e.g., hypermethylation, e.g., hypermethylation of CpG islands) in the gene's promoter region.

A combination of inhibitors described herein can increase the expression of a gene by at least about 10%, at least about 20%, at least about 30%, at least about 40%, at least about 50%, at least about 60%, at least about 70%, at least about 80%, at least about 90%, at least about 95%, at least about 99%, or at least about 100% as compared to the level of expression of the gene under identical conditions but in the absence of the combination and in the presence of an agent that decreases histone deacetylation. The gene can be, e.g., a gene that has been epigenetically silenced, e.g., by HDAC activity and/or by DNA methylation (e.g., hypermethylation, e.g., hypermethylation of CpG islands) in the gene's promoter region.

A SIRT1 inhibitor can be used in combination with more than one agent that decreases DNA methylation and/or more than one agent that decreases histone deacetylation. For example, a SIRT1 inhibitor can be used in combination with azacitidine and decitabine, two DNA methyltransferase inhibitors, for example, in the treatment of cancer, e.g., hematologic malignancies. As another example, MGCD0103 is an oral compound currently in multiple Phase I clinical trials in solid tumors and hematological malignancies and in one combination Phase I/II trial with Vidaza (azacitidine for injectable suspension) for high-risk myelodysplastic syndromes (MDS) and acute myelogenous leukemia (AML). In accordance with this disclosure, a SIRT1 inhibitor can be used in combination with one or both of MGCD0103 and/or azacitidine.

When the compositions of this disclosure involve a combination of a SIRT1 inhibitor and one or more additional agents, both the SIRT1 inhibitor and the additional agent should be present at dosage levels of between about 10 to 100%, e.g., between about 10 to 95% of the dosage normally administered in a monotherapy regimen.

Combination therapy can be advantageous, e.g., because the therapeutic effect achieved with the combination can be greater than the effect achieved by either agent alone. For example, the maximum dose of a first agent may be limited due to toxicity. Thus, the therapeutic effect achieved of that first agent is likewise limited. The same could be true for a second agent when administered alone. However, if the first agent is administered in combination with the second agent (both, e.g., at their maximum doses), and the two agents have an additive or synergistic effect, the total therapeutic effect achieved by the combination will be greater than that achieved with either agent alone. Similarly, if two agents have additive or synergistic effects when administered in combination, then, to achieve a given therapeutic effect (e.g., an effect that can be achieved by one of the agents when used alone), the doses required of each agent when used in combination can be less than the dose required if either of the agents was used alone. This decreased dose of each agent could, for example, result in decreased side effects or toxicity caused by one or both of the agents because less is administered.

Upon improvement of a patient's condition, a maintenance dose of a compound, composition or combination of this disclosure may be administered, if necessary. Subsequently, the dosage or frequency of administration, or both, may be reduced, e.g., to about ½ or ¼ or less of the dosage or frequency of administration, as a function of the symptoms, to a level at which the improved condition is retained when the symptoms have been alleviated to the desired level, treatment should cease. Subjects may, however, require intermittent treatment on a long-term basis upon any recurrence of disease symptoms.

It should also be understood that a specific dosage and treatment regimen for any particular subject will depend upon a variety of factors, including the activity of the specific compound employed, the age, body weight, general health, sex, diet, time of administration, rate of excretion, drug combination, and the judgment of the treating physician and the severity of the particular disease being treated. The amount of active ingredients will also depend upon the particular described compound and the presence or absence and the nature of the additional agent in the composition.

The SIRT1 inhibitor and second agent can be formulated into a pharmaceutical composition, either separately or together, for example, with one or more pharmaceutically acceptable carriers, adjuvants, or vehicles. Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions of this invention may be administered enterally (e.g., orally), parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the SIRT1 inhibitor and an additional agent (e.g., an inhibitor of epigenetic silencing described herein) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

Also within the invention is a patch to deliver the combinations described herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device.

In some cases (e.g., when dominant negative forms of SIRT1 and/or of HDACs and/or of DNA methyltransfersases) are used to practice the invention, these agents can be administered via gene therapy techniques (e.g., via adenoviral or adeno-associated virus delivery).

When the compositions of this invention comprise a combination of a SIRT1 inhibitor and a second agent (e.g., an inhibitor of epigenetic silencing), both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The term “mammal” includes organisms, which include mice, rats, cows, sheep, pigs, rabbits, goats, and horses, monkeys, dogs, cats, and preferably humans.

The term “treating” or “treated” refers to administering a compound(s) described herein to a subject with the purpose to cure, heal, alleviate, relieve, alter, remedy, ameliorate, improve, or affect a disease, e.g., cancer, the symptoms of the disease or the predisposition toward the disease.

A “therapeutically effective amount” or an amount required to achieve a “therapeutic effect” can be determined based on the effect of the administered agent(s). A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition is outweighed by the therapeutically beneficial effects.

The combination described herein can be administered, e.g., once or twice daily, or about one to four times per week, or preferably weekly, biweekly, or monthly, e.g., for between about 1 to 10 weeks (e.g., between 2 to 8 weeks or between about 3 to 7 weeks, or for about 4, 5, or 6 weeks) or for one, two three, four, five, six, seven, eight, nine, ten, eleven, twelve, or more months (e.g., for up to 24 months). The skilled artisan will appreciate that certain factors may influence the dosage and timing required to effectively treat a subject, including but not limited to the severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments. Animal models can also be used to determine a useful dose, e.g., an initial dose or a regimen.

In addition, after an administration period described herein with a combination described herein, a maintenance dose can be administered to the subject. For example, the maintenance dose can include a lower dose of one or both of the drugs of the combination described herein, a dose of only one of the drugs described herein (e.g., at the same or at a lower dose than in the initial administration period). As another example, if a combination of a SIRT1 inhibitor and an agent that decreases DNA methylation is used for the initial administration period, an agent that decreases histone deacetylation can be used alone for the maintenance dose, and vice versa. The maintenance dose may be administration of another combination described herein, e.g., a combination described herein but not employed in the initial administration period. For example, if a combination of a SIRT1 inhibitor and an agent that decreases DNA methylation is used for the initial administration period, a combination of a SIRT1 inhibitor and an agent that decreases histone deacetylation can be used for the maintenance dose, and vice versa. The maintenance dose can be administered, e.g., for a period of one, two three, four, five, six, seven, eight, nine, ten, eleven, twelve, or more months (e.g., for up to 24 or 36 months or longer) after termination of the initial administration period terminates.

An effective amount of the compound described above may range from about 0.1 mg/Kg to about 500 mg/Kg, alternatively from about 1 to about 50 mg/Kg. For example, an HDI, such as SAHA, can be administered in doses of 75, 150, 300, 600, and 900 mg/m²/day). For example, a dose of 300 mg/m²/day for 5 days for 3 weeks can be used, e.g., for hematological patients (see also Kelly et al. Clin. Cancer Res. 9:3578-3588 (2003)).

Effective doses will also vary depending on route of administration, as well as the co-administration with other agents, e.g., a second agent described herein.

### Antibodies

Exemplary agents that inhibit SIRT1, that decrease DNA methylation, or decrease histone deacetylation include antibodies that bind to (e.g., inhibit the activity of) SIRT1, DNA methyltransferases, or HDACs. In one embodiment, the antibody inhibits the interaction between the protein and its binding partner (e.g., an enzyme and its substrate), e.g., by physically blocking the interaction, decreasing the affinity of the protein for its binding partner, disrupting or destabilizing protein complexes, sequestering the protein, or targeting the protein for degradation. In one embodiment, the antibody can bind to the protein at one or more amino acid residues that participate in the binding interface between the protein and its binding partner. Such amino acid residues can be identified, e.g., by alanine scanning. In another embodiment, the antibody can bind to residues that do not participate in the binding. For example, the antibody can alter a conformation of the protein and thereby reduce binding affinity, or the antibody may sterically hinder binding. In other embodiments, the antibody can increase the activity (e.g., act as an agonist) of an agent that promotes DNA demethylase activity or increase the activity of an agent that promotes histone acetylation (e.g., histone acetyl transferase activity, e.g., histone acetyl transferases (HATs), e.g., PCAF, CBP, GCN5, p300/CREB, Esa1, Hat1, a protein of the p160 family, TAFII250, or Tip60).

As used herein, the term “antibody” refers to a protein that includes at least one immunoglobulin variable region, e.g., an amino acid sequence that provides an immunoglobulin variable domain or an immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)₂ fragments, Fd fragments, Fv fragments, and dAb fragments) as well as complete antibodies, e.g., intact and/or full length immunoglobulins of types IgA, IgG (e.g., IgG1, IgG2, IgG3, IgG4), IgE, IgD, IgM (as well as subtypes thereof). The light chains of the immunoglobulin may be of types kappa or lambda. In one embodiment, the antibody is glycosylated. An antibody can be functional for antibody-dependent cytotoxicity and/or complement-mediated cytotoxicity, or may be non-functional for one or both of these activities.

The VH and VL regions can be further subdivided into regions of hypervariability, termed “complementarity determining regions” (“CDR”), interspersed with regions that are more conserved, termed “framework regions” (FR). The extent of the FR's and CDR's has been precisely defined (see, Kabat, E. A., et al. (1991) Sequences of Proteins of Immunological Interest, Fifth Edition, U.S. Department of Health and Human Services, NIH Publication No. 91-3242; and Chothia, C. et al. (1987) J. Mol. Biol. 196:901-917). Kabat definitions are used herein. Each VH and VL is typically composed of three CDR's and four FR's, arranged from amino-terminus to carboxyl-terminus in the following order: FR1, CDR1, FR2, CDR2, FR3, CDR3, FR4.

An “immunoglobulin domain” refers to a domain from the variable or constant domain of immunoglobulin molecules. Immunoglobulin domains typically contain two β-sheets formed of about seven β-strands, and a conserved disulphide bond (see, e.g., A. F. Williams and A. N. Barclay (1988) Ann. Rev Immunol. 6:381-405). An “immunoglobulin variable domain sequence” refers to an amino acid sequence that can form a structure sufficient to position CDR sequences in a conformation suitable for antigen binding. For example, the sequence may include all or part of the amino acid sequence of a naturally-occurring variable domain. For example, the sequence may omit one, two, or more N- or C-terminal amino acids, internal amino acids, may include one or more insertions or additional terminal amino acids, or may include other alterations. In one embodiment, a polypeptide that includes an immunoglobulin variable domain sequence can associate with another immunoglobulin variable domain sequence to form a target binding structure (or “antigen binding site”), e.g., a structure that interacts with a target protein, e.g., SIRT1, an HDAC (e.g., HDAC1, HDAC2, HDAC3, HDAC8, HDAC11, HDAC4, HDAC5, HDAC6, HDAC7, or HDAC9), a DNA methyltransferase (e.g., DNMT1, DNMT2, DNMT3A, or DNMT3B), a DNA demethylase, or an histone acetyl transferase (HAT, e.g., PCAF, CBP, GCN5, p300/CREB, Esa1, Hat1, a protein of the p160 family, TAFII250, or Tip60).

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains. The heavy and light immunoglobulin chains can be connected by disulfide bonds. The heavy chain constant region typically includes three constant domains, CH1, CH2, and CH3. The light chain constant region typically includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

One or more regions of an antibody can be human, effectively human, or humanized. For example, one or more of the variable regions can be human or effectively human. For example, one or more of the CDRs, e.g., HC CDR1, HC CDR2, HC CDR3, LC CDR1, LC CDR2, and LC CDR3, can be human. Each of the light chain CDRs can be human. HC CDR3 can be human. One or more of the framework regions can be human, e.g., FR1, FR2, FR3, and FR4 of the HC or LC. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. One or more of the constant regions can be human, effectively human, or humanized. In another embodiment, at least 70, 75, 80, 85, 90, 92, 95, or 98% of the framework regions (e.g., FR1, FR2, and FR3, collectively, or FR1, FR2, FR3, and FR4, collectively) or the entire antibody can be human, effectively human, or humanized. For example, FR1, FR2, and FR3 collectively can be at least 70, 75, 80, 85, 90, 92, 95, 98, or 99% identical, or completely identical, to a human sequence encoded by a human germline segment.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified such that the modified form elicits less of an immune response in a human than does the non-modified form, e.g., is modified to include a sufficient number of human framework amino acid positions such that the immunoglobulin variable region does not elicit an immunogenic response in a normal human. Descriptions of “humanized” immunoglobulins include, for example, U.S. Pat. Nos. 6,407,213 and 5,693,762. In some cases, humanized immunoglobulins can include a non-human amino acid at one or more framework amino acid positions.

### Antibody Generation

Antibodies that bind to a target protein (e.g., SIRT1, an HDAC (e.g., HDAC1, HDAC2, HDAC3, HDAC8, HDAC11, HDAC4, HDAC5, HDAC6, HDAC7, or HDAC9), a DNA methyltransferase (e.g., DNMT1, DNMT2, DNMT3A, or DNMT3B), a DNA demethylase, or an histone acetyl transferase (HAT, e.g., PCAF, CBP, GCN5, p300/CREB, Esa1, Hat1, a protein of the p160 family, TAFII250, or Tip60)) can be generated by a variety of means, including immunization, e.g., using an animal, or in vitro methods such as phage display. All or part of the target protein can be used as an immunogen or as a target for selection. In one embodiment, the immunized animal contains immunoglobulin producing cells with natural, human, or partially human immunoglobulin loci. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al. (1994) Nat. Gen. 7:13-21; U.S. 2003-0070185; U.S. Pat. No. 5,789,650; and PCT Application WO 96/34096.

Non-human antibodies to the target proteins can also be produced, e.g., in a rodent. The non-human antibody can be humanized, e.g., as described in EP 239 400; U.S. Pat. Nos. 6,602,503; 5,693,761; and 6,407,213, deimmunized, or otherwise modified to make it effectively human.

EP 239 400 (Winter et al.) describes altering antibodies by substitution (within a given variable region) of their complementarity determining regions (CDRs) for one species with those from another. Typically, CDRs of a non-human (e.g., murine) antibody are substituted into the corresponding regions in a human antibody by using recombinant nucleic acid technology to produce sequences encoding the desired substituted antibody. Human constant region gene segments of the desired isotype (usually gamma I for CH and kappa for CL) can be added and the humanized heavy and light chain genes can be co-expressed in mammalian cells to produce soluble humanized antibody.

Other methods for humanizing antibodies can also be used. For example, other methods can account for the three dimensional structure of the antibody, framework positions that are in three dimensional proximity to binding determinants, and immunogenic peptide sequences. See, e.g., PCT Application WO 90/07861; U.S. Pat. Nos. 5,693,762; 5,693,761; 5,585,089; and 5,530,101; Tempest et al. (1991) Biotechnology 9:266-271 and U.S. Pat. No. 6,407,213. Still another method is termed “humaneering” and is described, for example, in U.S. 2005-008625.

Fully human monoclonal antibodies that bind to target proteins can be produced, e.g., using in vitro-primed human splenocytes, as described by Boerner et al. (1991) J. Immunol. 147:86-95. They may be prepared by repertoire cloning as described by Persson et al. (1991) Proc. Nat. Acad. Sci. USA 88:2432-2436 or by Huang and Stollar (1991) J. Immunol. Methods 141:227-236; also U.S. Pat. No. 5,798,230. Large non-immunized human phage display libraries may also be used to isolate high affinity antibodies that can be developed as human therapeutics using standard phage technology (see, e.g., Hoogenboom et al. (1998) Immunotechnology 4:1-20; Hoogenboom et al. (2000) Immunol Today 2:371-378; and U.S. 2003-0232333).

### Antibody and Protein Production

Antibodies and other proteins described herein can be produced in prokaryotic and eukaryotic cells. In one embodiment, the antibodies (e.g., scFv's) are expressed in a yeast cell such as Pichia (see, e.g., Powers et al. (2001) J. Immunol. Methods 251:123-35), Hanseula, or Saccharomyces.

Antibodies, particularly full length antibodies, e.g., IgG's, can be produced in mammalian cells. Exemplary mammalian host cells for recombinant expression include Chinese Hamster Ovary (CHO cells) (including dhfr-CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NS0 myeloma cells and SP2 cells, COS cells, K562, and a cell from a transgenic animal, e.g., a transgenic mammal. For example, the cell is a mammary epithelial cell.

In addition to the nucleic acid sequence encoding the immunoglobulin domain, the recombinant expression vectors may carry additional nucleic acid sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216; 4,634,665; and 5,179,017). Exemplary selectable marker genes include the dihydrofolate reductase (DHFR) gene (for use in dhfr⁻ host cells with methotrexate selection/amplification) and the neo gene (for G418 selection).

In an exemplary system for recombinant expression of an antibody (e.g., a full length antibody or an antigen-binding portion thereof), a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector can also carry a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, to transfect the host cells, to select for transformants, to culture the host cells, and to recover the antibody from the culture medium. For example, some antibodies can be isolated by affinity chromatography with a Protein A or Protein G.

Antibodies (and Fc fusions) may also include modifications, e.g., modifications that alter Fc function, e.g., to decrease or remove interaction with an Fc receptor or with C1q, or both. For example, the human IgG1 constant region can be mutated at one or more residues, e.g., one or more of residues 234 and 237, e.g., according to the numbering in U.S. Pat. No. 5,648,260. Other exemplary modifications include those described in U.S. Pat. No. 5,648,260.

For some proteins that include an Fc domain, the antibody/protein production system may be designed to synthesize antibodies or other proteins in which the Fc region is glycosylated. For example, the Fc domain of IgG molecules is glycosylated at asparagine 297 in the CH2 domain. The Fc domain can also include other eukaryotic post-translational modifications. In other cases, the protein is produced in a form that is not glycosylated.

Antibodies and other proteins can also be produced by a transgenic animal. For example, U.S. Pat. No. 5,849,992 describes a method for expressing an antibody in the mammary gland of a transgenic mammal. A transgene is constructed that includes a milk-specific promoter and nucleic acid sequences encoding the antibody of interest, e.g., an antibody described herein, and a signal sequence for secretion. The milk produced by females of such transgenic mammals includes, secreted-therein, the protein of interest, e.g., an antibody or Fc fusion protein. The protein can be purified from the milk, or for some applications, used directly.

Methods described in the context of antibodies can be adapted to other proteins, e.g., Fc fusions and soluble receptor fragments.

### Kits

The compounds (e.g., a SIRT1 inhibitor and second agent) described herein can be provided in a kit. The kit includes (a) the compounds described herein, e.g., a composition(s) that includes a compound(s) described herein, and, optionally (b) informational material. The informational material can be descriptive, instructional, marketing or other material that relates to the methods described herein and/or the use of a compound(s) described herein for the methods described herein.

The informational material of the kits is not limited in its form. In one embodiment, the informational material can include information about production of the compound, molecular weight of the compound, concentration, date of expiration, batch or production site information, and so forth. In one embodiment, the informational material relates to methods for administering the compound.

In one embodiment, the informational material can include instructions to administer a compound(s) (e.g., the combination of a SIRT1 inhibitor and second agent) described herein in a suitable manner to perform the methods described herein, e.g., in a suitable dose, dosage form, or mode of administration (e.g., a dose, dosage form, or mode of administration described herein). In another embodiment, the informational material can include instructions to administer a compound(s) described herein to a suitable subject, e.g., a human, e.g., a human having or at risk for a disorder described herein, e.g., cancer, e.g., breast or colon cancer.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In addition to a compound(s) described herein, the composition of the kit can include other ingredients, such as a solvent or buffer, a stabilizer, a preservative, a flavoring agent (e.g., a bitter antagonist or a sweetener), a fragrance or other cosmetic ingredient, and/or a second agent for treating a condition or disorder described herein. Alternatively, the other ingredients can be included in the kit, but in different compositions or containers than a compound described herein. In such embodiments, the kit can include instructions for admixing a compound(s) described herein and the other ingredients, or for using a compound(s) described herein together with the other ingredients, e.g., instructions on combining the two agents prior to administration.

A compound(s) described herein can be provided in any form, e.g., liquid, dried or lyophilized form. It is preferred that a compound(s) described herein be substantially pure and/or sterile. When a compound(s) d described herein is provided in a liquid solution, the liquid solution preferably is an aqueous solution, with a sterile aqueous solution being preferred. When a compound(s) described herein is provided as a dried form, reconstitution generally is by the addition of a suitable solvent. The solvent, e.g., sterile water or buffer, can optionally be provided in the kit.

The kit can include one or more containers for the composition containing a compound(s) described herein. In some embodiments, the kit contains separate containers (e.g., two separate containers for the two agents), dividers or compartments for the composition(s) and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

### EXAMPLES

The class III histone deactylase (HDAC), SIRT1, has cancer relevance because it can regulate lifespan in multiple organisms, down-regulate p53 function through deacetylation, and is linked to polycomb gene silencing in Drosophila. However, it has not been reported to mediate heterochromatin formation or heritable silencing for endogenous mammalian genes. Herein, it is shown that SIRT1 localizes to promoters of several aberrantly silenced tumor suppressor genes (TSGs) in which 5′ CpG islands are densely hypermethylated, but not to these same promoters in cell lines in which the promoters are not hypermethylated and the genes are expressed. Heretofore, only type I and II HDACs, through deactylation of lysines 9 and 14 of histone H3 (H3-K9 and H3-K14, respectively), had been tied to the above TSG silencing. However, inhibition of these enzymes alone fails to re-activate the genes unless DNA methylation is first inhibited.

In contrast, as shown herein, inhibition of SIRT1 by pharmacologic, dominant negative, and siRNA (small interfering RNA)-mediated inhibition in breast and colon cancer cells causes increased H4-K16 and H3-K9 acetylation at endogenous promoters and gene re-expression despite full retention of promoter DNA hypermethylation. Furthermore, SIRT1 inhibition affects key phenotypic aspects of cancer cells. Thus, the data presented herein demonstrated a new component of epigenetic TSG silencing that may potentially link some epigenetic changes associated with aging with those found in cancer, and provide new directions for therapeutically targeting these important genes for re-expression. (Pruitt et al., PLOS Genetics 2:0344-0352 (2006)).

### Example 1

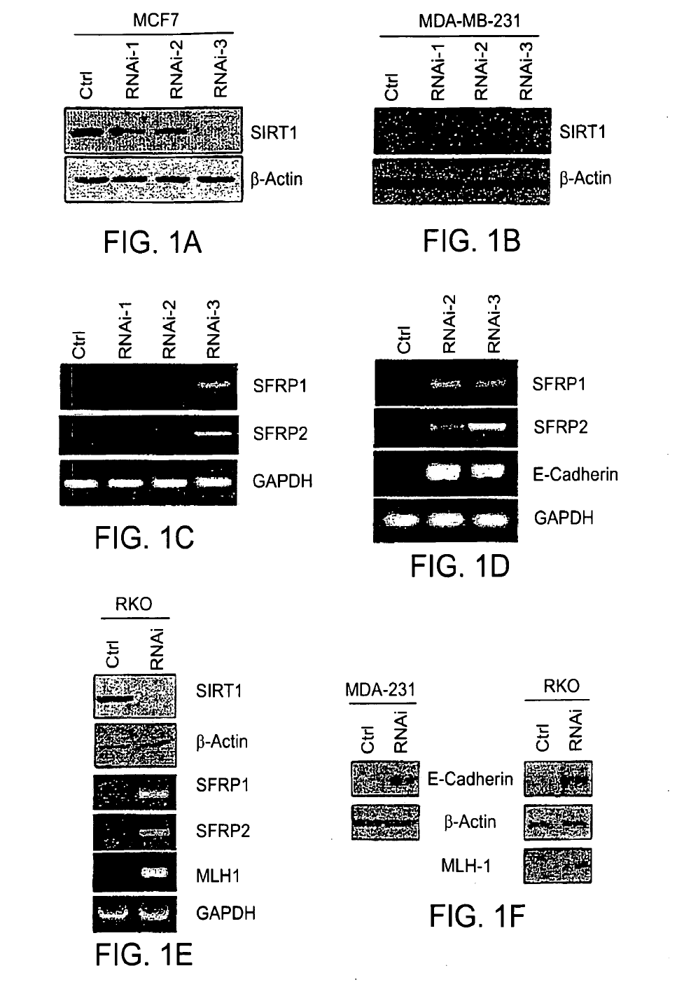

To determine whether SIRT1 specifically plays a role in silencing TSGs whose promoters have 5′ CpG islands that are densely hypermethylated, first screens using RNA-interference (RNAi) to disrupt the function of this protein were applied and the effects on the targets were evaluated. Both breast and colon cancer cell lines were chosen for the study, and several RNAi sequences targeting SIRT1 specifically were tested for their efficacy. SIRT1 protein levels in both MCF7, shown in FIG. 1A, and MDA-MB-231, shown in FIG. 1B, breast cancer cells were reduced via retroviral infection with a pSuper-retro-RNAi construct encoding short hairpin loop RNA (shRNA) specific for “knocking down” SIRT1. Three RNAi constructs were tested, and the sequence termed RNAi-3 yielded the greatest knockdown in MCF7 (FIG. 1A), whereas both RNAi-2 and RNAi-3 were very effective in reducing protein levels in MDA-MB-231 cells, as shown in FIG. 1B. Since cells were infected with equivalent titers of virus encoding the shRNAs, it is unclear why RNAi-3 was the most effective, but as shown below, the degree of knockdown served as a good control since it correlates very well with effects on gene re-expression.

Correlating with the knockdown pattern of SIRT1 in each cell type, a re-expression of key TSGs that are frequently epigentically silenced in a number of different cancers was observed. The anti-tumor genes identified all have promoter DNA hypermethylation, and they have important anti-tumor functions ranging from mediating proper epithelial cell differentiation to promoting cell-cell adhesion. The genes include members of the family of secreted frizzled-related proteins (SFRP1 and SFRP2), which are frequently epigenetically inactivated during colon and breast cancer progression, and contribute to aberrant activation of Wnt signaling, shown in FIGS. 1C and 1D [6,28]. Additionally, SIRT1 was found to maintain silencing of E-cadherin, a gene mediating cell-cell adhesion that is also inactivated epigenetically in many cancers (FIG. 1D) [29-31]. Finally, SIRT1 protein levels were also reduced in RKO colon cancer cells and SIRT1 was found to maintain silencing of TSGs including the mismatch repair gene, MLH 1 (FIG. 1E), for which epigenetic silencing and loss of function produces the microsatellite instability (MIN+) colon cancer phenotype [32,33]. Additionally, it was found that the transcription factors encoding GATA-4 and GATA-5 genes, whose promoter DNA is hypermethylated [34], were also re-expressed in both colon and breast cancer cells (data not shown).

To further determine whether the gene re-expression with this very specific approach for SIRT1 inhibition leads to protein re-expression, parallel Western blots on samples for which proven antibodies are available were performed. Consistent with gene re-expression, there was found restoration of E-cadherin protein in breast and colon cancer cell lines and MLH1 in colon cancer lines in which these genes are hypermethylated and silenced (FIG. 1F). These findings further demonstrate that SIRT1 specifically, and substantially, contributes to the aberrant heritable silencing of this panel of TSGs. Moreover, the levels of gene expression when SIRT1 function is reduced is similar to that observed for these genes when moderate doses of 5′-aza-deoxycytidine (Aza) is employed to achieve promoter demethylation [32,35]. Furthermore, the data have demonstrated previously that the degree of protein re-expression for MLH1 obtained correlates with restored protein function in RKO cells [32].

The panels in FIG. 1 are as follows. FIG. 1(A) shows that RNAi-3 is most effective for reduction of SIRT1 in MCF7 cells. Retroviral expression vectors encoding SIRT1 cDNA that produce short hairpin loop RNA targeting either distinct regions of SIRT1 mRNA (RNAi-1, -2, or -3) or a control (ctrl) were used to infect MCF7. Western blot analysis for SIRT1 and β-actin was performed 48 hours after two rounds of infection. FIG. 1(B) shows that both RNAi-2 and -3 are effective for reduction of SIRT1 protein in MDA-MB-231 cells as described in FIG. 1(A). FIG. 1(C) demonstrates that SIRT1 inhibition leads to TSG re-expression in MCF7 cells. RNA was isolated from parallel samples analyzed in FIG. 1(A), and RT-PCR was performed with intron-spanning primers specific for the genes SFRP1 and SFRP2. GAPDH was also analyzed as a control. Only the shRNA (RNAi-3) that caused substantial reduction in SIRT1 protein led to gene re-expression. Control samples in which no reverse transcriptase was added were analyzed separately, and all were negative for amplification of the indicated genes. FIG. 1(D) shows that SIRT1 inhibition leads to TSG re-expression in MDA-MB-231 cells. RT-PCR was performed for analysis of the genes SFRP1, SFRP2, and E-cadherin as described in FIG. 1(A). Only the shRNAs (RNAi-2 and -3) that caused substantial reduction in SIRT1 protein led to gene re-expression. FIG. 1(E) demonstrates that SIRT1 inhibition leads to TSG re-expression in RKO cells. SIRT1 protein reduction by RNAi-3 (top panel) as described in FIG. 1(A) leads to gene re-expression of SFRP1, SFRP2, and MLH1 as described in FIG. 1(C). FIG. 1(F) shows that MDA-MB-231 and RKO cells infected with control or RNAi-3 shRNA as described in FIG. 1(A) were selected with puromycin for 3 days and pooled colonies were harvested for Western blot analysis of protein re-expression that corresponded with the gene reactivation described in FIGS. 1(D) and (E).

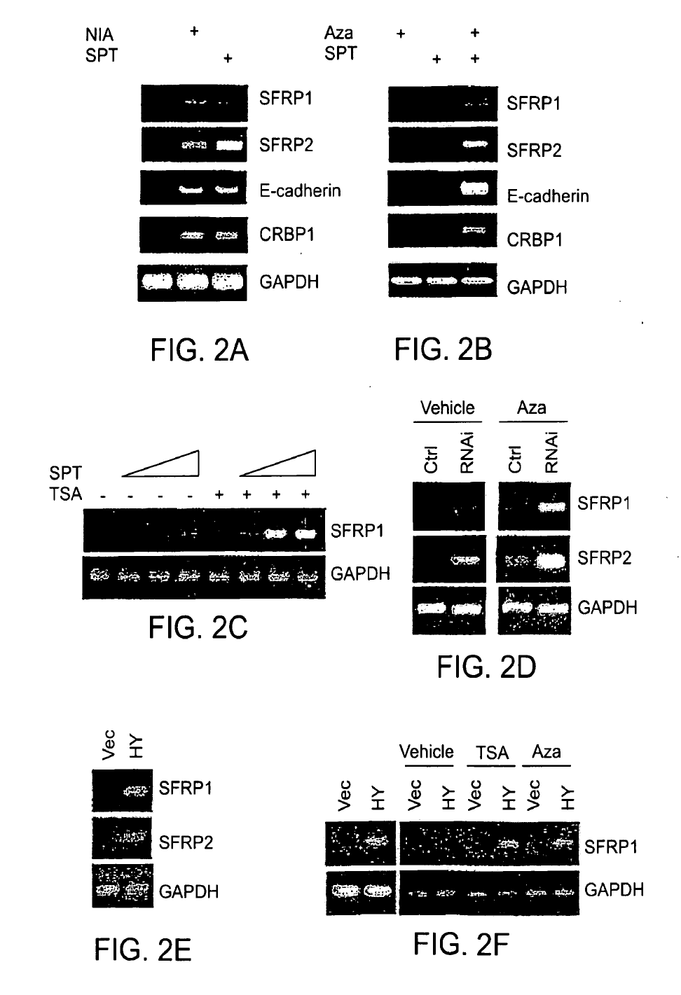

To further assess the role SIRT1 plays in silencing TSGs whose promoter DNA is hypermethylated, two additional approaches were used. One is a pharmacologic approach using the general sirtuin inhibitor, nicotinamide (NIA) [12,36], and the more sir2-specific inhibitor, splitomicin (SPT) [13,37]. Consistent with the above RNAi data, it was found that these sirtuin inhibitors could cause the re-expression of the epigenetically silenced, hypermethylated TSGs studied above, and another such gene, CRBP1, in the human breast cancer cell lines MDA-MB-231 (FIG. 2) or MCF7 (data not shown). Using yet a third approach to assess the role that SIRT1 plays, a catalytically inactive, dominant negative inhibitor of SIRT1, SIRT1H363Y [21] was expressed, and screened representative genes to further validate the specific involvement of this protein in repression of the described panel of hypermethylated TSGs. In both MCF7 and MDA-MB-231 breast cancer cells in which SIRT1H363Y was expressed through retroviral infection, there was observed a re-expression of SFRP1 and SFRP2 (FIGS. 2E and 2F [left panel]). Additionally, the same effect for GATA-4 in HCT116 colon cancer cells when the H363Y mutant was expressed, but not the wild type (data not shown) was observed.

Strikingly, there was a synergy in gene activation by combining the class VII HDI, TSA, with increasing doses of SPT to reactivate genes whose promoters have hypermethylated DNA (FIG. 2C and data not shown). To again assess the synergy with DNA demethylation, low titers of shRNA retrovirus and low-dose Aza were used, and there was a synergistic re-expression of SFRP1 and SFRP2 (FIG. 2D). The specific contribution of SIRT1 inhibition to the synergistic effects of combining either Aza treatment or TSA with sirtuin inhibition was investigated using low titers of SIRT1H363Y retrovirus. The result observed was the synergistic reactivation of SFRP1 (FIG. 2F, right panel), and GATA-5 and SFRP2 (data not shown) in response to inhibition with the SIRT1 dominant negative SIRT1H363Y (HY) when used in low titers and combined with either Aza or TSA. These results provide strong evidence that, although SIRT1 inhibition alone is sufficient for the reactivation of our panel of TSGs, inhibition of DNA methylation and type I/II HDACs can cooperate with SIRT1 inhibition in such reactivation.

The panels of FIG. 2 are as follows. FIG. 2(A) shows the results of experiments demonstrating that pharmacologic inhibition of SIRT1 causes TSG re-expression. MDA-MB-231 cells were treated with 15 mM NIA or 300 μM SPT for 21 hours, RNA was isolated, and RT-PCR was performed with intron-spanning primers specific for the indicated genes. Control samples in which no reverse transcriptase was added were analyzed separately, and all were negative for amplification of the indicated genes. FIG. 2(B) demonstrates that the combined treatment with low doses of Aza and splitomicin (SPT) synergizes in the re-expression of TSGs. MDA-MB-231 cells were treated with either 50 nM Aza (+), 100 μM SPT (+) or with both Aza and SPT (++), and 34 hours later, RT-PCR was performed for the indicated genes as described in FIG. 2(A). FIG. 2(C) shows that the combined treatment with SPT and TSA synergize in the re-expression of genes. MDA-MB-231 cells were treated with either 0, 50, 100, or 120 μM SPT alone for 34 hours, or the treatment was followed by treatment with 300 nM TSA for 3 hours prior to RNA isolation and RT-PCR analysis. The results in FIG. 2(D) demonstrate that SIRT1 protein knockdown synergizes with low doses of Aza for gene re-expression. MDA-MB-231 cells were infected with low titers of virus for shRNA specific for SIRT1. Aza (100 nM) was added 24 hours prior to RNA isolation, and RT-PCR analysis was performed for the genes SFRP 1, SFRP2, and GAPDH as described in FIG. 2(A). The results in FIG. 2(E) show that dominant negative inhibition of SIRT1 leads to TSG re-expression in MCF7 cells. MCF7 cells were infected with virus encoding either pBabe (vec) or the catalytically inactive SIRT1H363Y (HY) mutant, and RT-PCR was performed as described in FIG. 2(A). FIG. 2(F) shows that dominant negative inhibition of SIRT1 leads to TSG re-expression and synergizes with TSA and Aza. As shown in the left panel, MDA-MB-231 cells were infected with a control (vec) or mutant SIRT1 virus (HY), and RT-PCR was performed as described in FIG. 2(A). MDA-MB-231 cells were infected with low titers of pBabe or pBabe-SIRT1H363Y retrovirus and subsequently treated with 100 nM Aza for 24 hours or with 300 nM TSA for 3 hours prior to harvest, and RT-PCR was performed.

As discussed earlier, it has been previously demonstrated that DNA methylation and histone deacetylation, involving class I and II HDACs, act as synergistic layers for TSG silencing in cancer and that inhibition of DNA methylation is dominant relative to the inhibition of deacetylation [6]. Thus, it was investigated whether disruption of sirtuin function could collaborate with either inhibitors of DNA methylation or type I/II HDIs in TSG re-expression. In this regard, low doses of Aza (50 nM) or SPT (50 μM) that were ineffective as single agents could be combined to achieve synergistic re-expression of the gene panel as shown by representative genes in FIG. 2B.

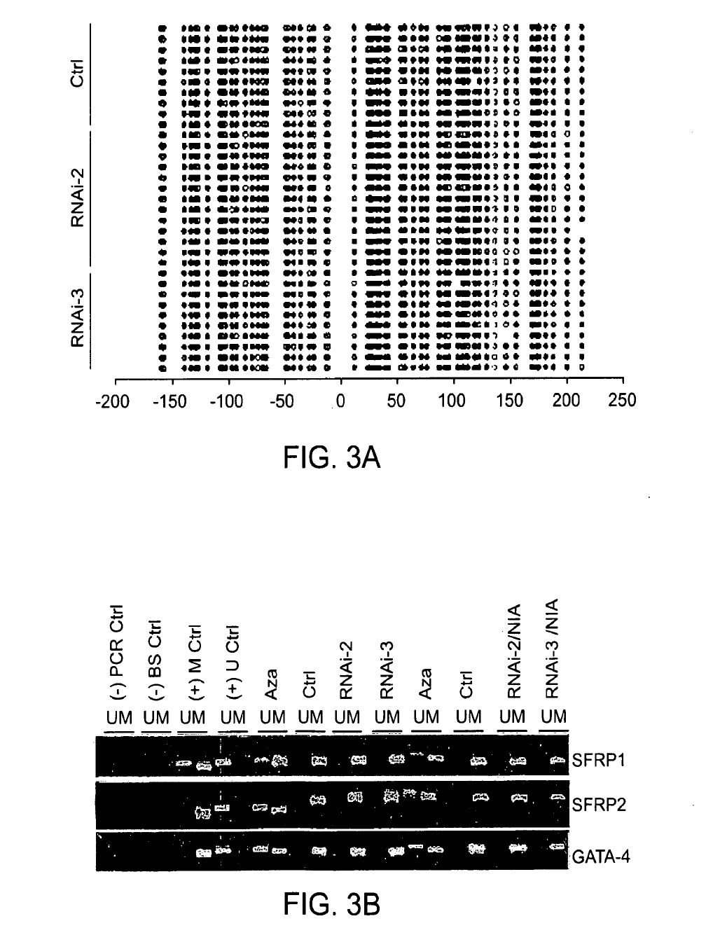

Given that SIRT1 appears to be intimately involved in maintaining silencing of the genes under study whose promoter DNA is densely hypermethylated, we wanted to determine whether the mechanism of reactivation coincided with any changes in the DNA methylation status at the re-expressed TSG promoters. To assess this, extensive bisulfite sequencing of samples in which TSGs were reactivated by transient knockdown of SIRT1 by RNAi was performed, as shown in FIG. 1 and by stable knockdown of SIRT1. There was observed no change in promoter methylation of SFRP1 or GATA-5 (FIG. 3A, S1, and S2). Moreover, a very sensitive, methylation-specific PCR (MSP) approach for detection of methylation status [38] yielded identical results (FIG. 3B) to those from bisulfite sequencing. In all previous studies of these genes, a similar degree of reactivation with Aza is always accompanied by significant promoter demethylation as assessed by MSP analyses or bisulfite sequencing [28,34]. Furthermore, when the cells with stable RNAi knockdown were treated with NIA to further inhibit any remaining SIRT1 protein, as shown in the RNAi-2/NIA and RNAi-3/NIA lanes in FIG. 3B, no restoration to the unmethylated state for genes examined was observed, even though they were re-expressed. Thus, it appears that SIRT1 inhibition alone is sufficient for the reactivation of tested TSGs even when dense promoter DNA methylation is maintained.

The panels of FIG. 3 are as follows. FIG. 3(A) shows the results of experiments performed to demonstrate that TSG re-expression occurs without changes in the methylation profile of multiple clones analyzed for SFRP1 promoter methylation. Parallel samples analyzed in FIG. 1D were subjected to bisulfite sequencing of the SFRP1 promoter from MDA-MB-231 cells stably infected with control vector or RNAi-2 or RNAi-3 retrovirus. Open circles indicate unmethylated cytosines, and closed circles indicate methylated cytosines. Numbers at the bottom show the position of cytosines relative to the transcription start site, which is at position 0, and those with a minus sign (−) are upstream from this start site. The region sequenced encompasses the CpG island in which methylation status correlates with gene expression status. FIG. 3(B) shows the results of MSP analyses of DNA from MDA-MB-231 cells stably expressing vector control, RNAi-2, or RNAi-3 retrovirus. From left to right: (−) PCR Ctrl indicates H₂O only; (−) BS ctrl indicates bisulfite-treated H₂0; (+) M ctrl indicates the cell line in which SFRP1 is partially methylated and SFRP2 and GATA4 are fully methylated; and (+) U ctrl indicates the Tera-2 cell line in which each gene is unmethylated. All remaining lanes are for MDA-MB-231. From left to right: Aza indicates 1 μM Aza (24 hours) treatment; Ctrl indicates empty vector infection; RNAi-2 indicates shRNA-2 infection alone; RNAi-3 indicates shRNA-3 infection alone; Aza indicates 1 μM Aza (24 hours treatment of control cells; Ctrl indicates empty vector infection+vehicle; RNAi-2 indicates shRNA-2 infection+5 mM NIA treatment; and RNAi-3 indicates shRNA-3 infection+5 mM NIA treatment.

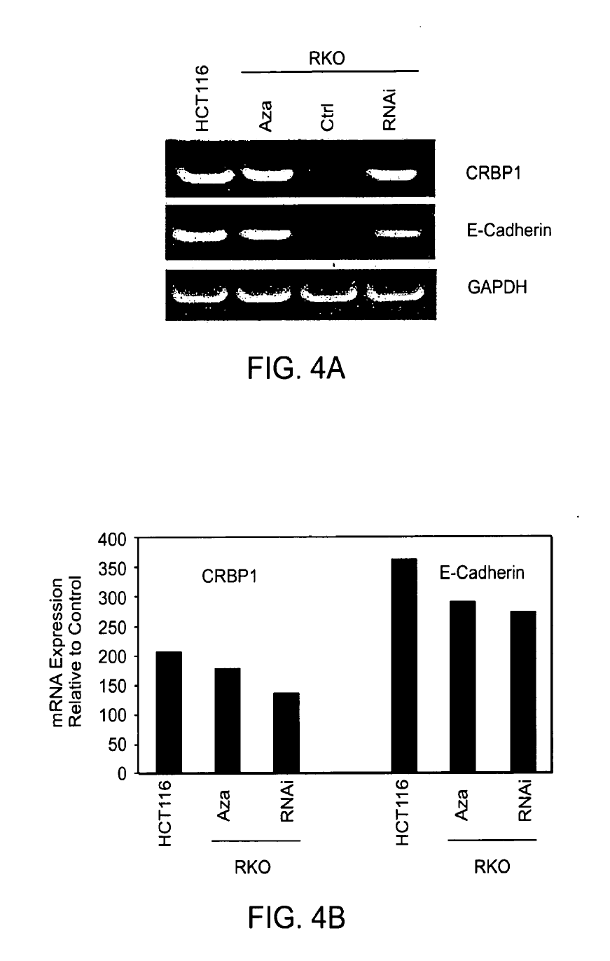

One question that emerges with the above re-expression of genes induced by SIRT1 reduction in the face of retained DNA methylation is how the extent of transcription achieved compares to expression of these genes when DNA methylation alone is markedly reduced or absent. To examine this, RT-PCR (FIG. 4A) and by quantitative real-time RT-PCR (FIG. 4B) was used to compare the re-expression achieved by SIRT1 knockdown of two genes with the basal expression of these same genes in an another cancer cell line in which the promoter DNA is not hypermethylated (FIG. 4). In RKO cells in which SIRT1 protein levels were reduced via shRNA, and the residual SIRT1 protein was inhibited with SPT, we observed a restoration of CRBP1 and E-cadherin mRNA transcripts to about 60%-75% of the levels for their basal expression in HCT116 cells in which the promoter DNA is not hypermethylated. Similarly, levels of re-expression of the genes after SIRT1 reduction were comparable to those achieved after decreased DNA methylation using intermediate doses of Aza (500 nM) (FIG. 4). These results provide evidence that SIRT1 inhibition plays a significant role in TSG re-expression even when promoter DNA methylation is retained and that SIRT1 likely cooperates with factors other than DNA methylation to help mediate the gene silencing.

FIG. 4 shows the following. FIG. 4(A) shows the results from experiments performed in RKO cells that were infected and stably selected to express short hairpin loop RNA targeting either a region unique to SIRT1 mRNA or a control (ctrl). To inhibit any residual SIRT1 protein, remaining RNAi-expressing cells were treated with 700 μM SPT and control samples were treated with DMSO for 24 hours. For comparison, control RNA was isolated from parallel samples from HCT116 cells in which the two genes under study, CRB1 and E-cadherin, do not have promoter DNA hypermethylation and are basally expressed. RKO cells were also treated with 0.5 μM Aza (24 hours), and samples were analyzed as described in FIG. 1A; RT-PCR was performed with intron-spanning primers specific for the two genes. GAPDH was also analyzed as a control. Only the shRNA (RNAi-3) that caused substantial reduction in SIRT1 protein leads to gene re-expression. Control samples in which no reverse transcriptase was added were analyzed separately, and all were negative for amplification of the indicated genes. FIG. 4(B) shows the results after parallel samples described above were analyzed using real-time quantitative PCR. The level of TSG re-expression induced by Aza treatment or SIRT1 inhibition as described in FIG. 4(A) was compared to levels of expression in HCT116 cells in which the TSGs are basally expressed.

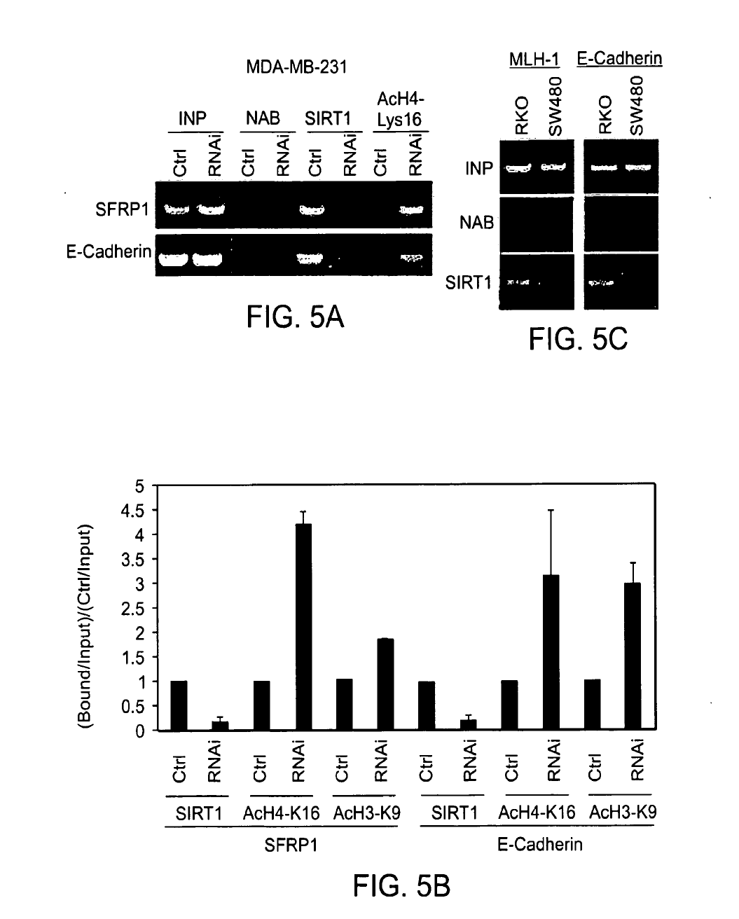

Next, it was examined whether SIRT1 localizes to the promoters of the hypermethylated genes studied and directly modulates histone changes. Chromatin immunoprecipitation (ChIP) assays in MDA-MB-231 cells were performed and SIRT1 localization at DNA-hypermethylated and silenced promoters for SFRP1, E-cadherin, and GATA-5 (FIG. 5 and data not shown) and at the silenced MLH1 and E-cadherin promoters in RKO colon cancer cells (FIG. 5C) was observed. This localization was reduced with shRNA knockdown of SIRT1 (FIG. 5A). Importantly, SIRT1 was absent from the promoters of the genes such as MLH1 and E-cadherin when their promoter DNA is not hypermethylated and the genes are basally expressed in the SW480 colon cancer cells (FIG. 5C).

Next, it was examined how modifications of lysine residues known to be associated with transcriptional repression mapped with SIRT1-associated gene silencing. During SFRP1 reactivation, and concurrent with shRNA knockdown of SIRT1, we observed robust increases in acetylation of H4-K16 (FIGS. 5A and 5B) which has been documented as a direct target of SIR2 in yeast [39-41] and a preferential target in human cells for an introduced SIRT1 induction reporter system [11]. Additionally, we observed significant increases in the levels of H4-K16 acetylation at the SFRP1, E-cadherin, and GATA-5 promoters (FIGS. 5A and 5B, and data not shown). A modest increases in H3-K9 acetylation at the SFRP1 promoter and more substantial increases in H3-K9 acetylation at the E-cadherin promoter (FIG. 4B). This latter modification has been tied to control by both class I and II HDACs, and SIRT1 [7,42].

The results are shown in FIG. 5. FIG. 5(A) shows the results of experiments in which pooled populations of MDA-MB-231 cells stably selected to express RNAi constructs were analyzed via ChIP. These samples were isolated in parallel to those analyzed in FIG. 3B. ChIP was performed with antibodies against SIRT1, acetylated histone H4, lysine 16 (H4-K16), or with no antibody (NAB) controls. Each promoter sequence was amplified by PCR under linear conditions for the genes SFRP1 and E-cadherin. The results in FIG. 5(B) show the average change in SIRT1 localization, acetylation of H4-K16, and acetylation of H3K9 at the SFRP1 and E-cadherin promoters as measured by ChIP based on quantification from multiple experiments. Error bars indicate the standard deviation for multiple experiments. FIG. 5(C) shows that SIRT1 localizes to the promoters of silent genes whose DNA is hypermethylated, but not to these same promoters in cells in which the genes are expressed. ChIP was performed with antibodies against SIRT1 in RKO and SW480 colon cancer cells. As shown in the left panel, SIRT1 localizes to the MLH1 promoter in RKO cells in which the gene is silent, but not to the MLH1 promoter in SW480 cells in which it is expressed. As shown in the right panel, SIRT1 localizes to the E-cadherin promoter in RKO cells in which the gene is silent, but not to the E-cadherin promoter in SW480 cells where it is expressed.

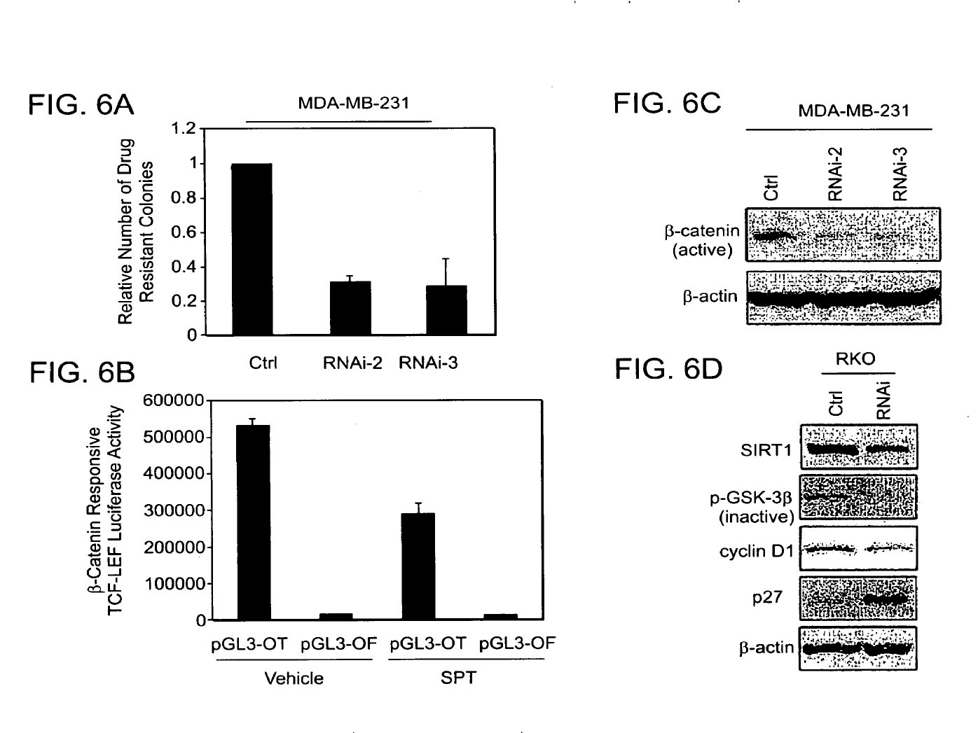

Finally, from an overall cellular phenotype, we might predict that, if SIRT1 is involved in the repression of TSGs, inhibiting its function and concomitant re-expression of such genes should affect cell growth and/or viability. The numbers of DNA-hypermethylated and silenced TSGs in the cancer cell lines under examination make a direct analysis of this difficult. However, the effects of SIRT1 on a series of colon and breast cancer phenotypic characteristics that would be predicted to change dramatically with re-expression of the TSGs under study were tested. First, we examined the numbers of drug-resistant colonies that are formed during drug selection of cells for stable siRNA (small interfering RNA) knockdown of SIRT1. As shown in FIG. 6A, a sharp reduction in cell colonies during such selection was observed.

Although the re-expression of many genes could account for the type of phenotypic change shown above, there is the possibility that reactivation of SFRP genes might be involved. Previously it has been shown that the silencing of the SFRP1 and -2 genes is important for aberrant activation of the Wnt pathway in colon cancer cells, and their re-introduction into such cells in which the genes are silenced causes sharp down-regulation of Wnt pathway function and apoptosis. [28]. First, we tested for the possible impact of the re-expression of these genes in colon cancer cells by examining key parameters of the Wnt signaling pathway following SIRT1 inhibition. A 50% reduction in the activation of a β-catenin-responsive TCF reporter construct, a canonical readout for Wnt pathway activity in colon cancer cells [28,43,44] with SPT treatment of RKO colon cancer cells was observes, as shown in FIG. 5B. Additionally, we found a 50% reduction in the activation of a β-catenin-responsive cyclin-D1 promoter reporter construct [45,46] with SPT treatment of RKO cells (data not shown). The data also demonstrated suppression of other Wnt pathway signaling parameters in that there was a decrease in inactive phospho-GSK-3β, a member of the β-catenin destruction complex, and a reduction in cyclin D1 levels, a downstream target of nuclear β-catenin (FIG. 6). The data also showed that inhibition of SIRT1 lead to increases in p27 protein levels in RKO cells, an observation consistent with another report [47] using dominant negative inhibition of SIRT1 in another cell type. As demonstrated in FIGS. 1 and 2 in breast cancer cells, SIRT1 is involved in the silencing of SFRP 1 and -2. Moreover, MDA-MB-231 cells express the wnt7b oncogene [48]. In MDA-MB-231 cells in which SIRT1 was inhibited stably by RNAi, we observed a sharp reduction in the levels of unphosphorylated or active β-catenin (FIG. 5B). Thus, SIRT1 inhibition causes re-expression of SFRPs that antagonize WNT signaling. Furthermore, SIRT1 inhibition causes re-expression of the E-cadherin gene, whose protein product complexes with β-catenin, and this gene reactivation collectively may suppress the constitutive activation of the WNT signaling pathway.

The panels of FIG. 6. FIG. 6(A) shows the results of experiments in which MDA-MB-231 cells were infected for two rounds with RNAi-2 and -3 retrovirus, and puromycin-resistant colonies were counted after 3 days of selection. Error bars indicate standard deviation from the average of three experiments. FIG. 6(B) provides the results obtained from experiments in which RKO cells were transfected with 500 ng of pGL3-OT, a TCF-LEF-responsive reporter, or pGL3-OF, a negative control with a mutated TCF-LEF binding site in combination with 10 ng of pRL-CMV vector. Twenty-four hours post-transfection, cells were treated with either vehicle (DMSO) control or with 700 μM SPT for 24 hours. Firefly luciferase activity was measured and normalized to the Renilla luciferase activities. The experiments shown in FIG. 6(C) were performed as described in FIG. 6(A), in which pooled populations of MDA-MB-231 cells stably expressing RNAi-2 or RNAi-3 were harvested, protein concentrations were determined, and Western blot analysis was performed. An antibody that specifically recognizes the unphosphorylated (active) form of β-catenin was used, and on the same blot, β-actin was probed to ensure equal loading. FIG. 6(D) shows the results from a Western blot analysis that was performed on RKO cells expressing control or SIRT1 RNAi. Antibodies against SIRT1, phospho-GSK3β (inactive), cyclin D1, p27, and β-actin were used for Western blotting. On the same blot, β-actin was probed to ensure equal loading.

### Methods

Cell culture and retroviral infection. MDA-MB-231, MCF7, HCT116, SW480 RKO, and Phoenix cells (ATCC, Rockville, Md., United States) were cultured in Dulbecco's modified Eagle's medium supplemented with 10% fetal bovine serum and 1% penicillin/streptomycin (Invitrogen, Carlsbad, Calif., United States). Retroviral infection was performed using either single or multiple rounds of infection. Briefly, Phoenix cells were transfected with either pBabe, pBabe-SIRT1H363Y, pSUPERretro, pSUPERretro-SIRT1-RNAi-1-3 (NM_{—}012238 positions 410, 589, and 1091; Oligo Engine, Seattle, Wash., United States) using Lipofectamine 2000 (Invitrogen). After 48 h of transfection, the medium containing retrovirus was collected, filtered, and supplemented with Polybrene prior to infection of target cells (MDA-MB-231, MCF7, or HCT116). Infected cells were either harvested 24-48 hours later or subjected to selection with 2-3 μg/ml puromycin for a week prior to harvest and analysis.

RNA and protein preparation and analysis. Total RNA was extracted (Invitrogen) according to the manufacturer's instructions and subjected to reverse transcription followed by both quantitative real-time and semi-quantitative polymerase chain reaction. For quantitative real-time analyses, the QuantiTect SYBR Green PCR kit (Qiagen, Valencia, Calif., United States) was used and the amplification conditions consisted of an initial 10-minutes denaturation step at 95° C., followed by 40 cycles of denaturation at 95° C. for 15 s and annealing and extension for 30 seconds and 60 seconds, respectively. A BioRad iCycler was used (BioRad, Hercules, Calif., United States), and for quantitation the comparative cycle threshold (Ct) method was used, normalizing the Ct values for the indicated gene to the Ct values of GAPDH relative to a control sample. For conventional PCR, at least two independent sets of intron-spanning primers [28,34,56] were used for the analysis of multiple genes, such as CRBP1, (NM_{—}002899), E-cadherin, (L34545), SFRP1, (BC036503), SFRP2, (BC008666), and Gata-4 (L34357). For Western blots, cells stably expressing RNAi constructs were harvested in 50 mM Tris-HCl, 1% NP-40, 0.25% sodium deoxycholate, 150 mM NaCl, 50 mM sodium fluoride, 1 mM dithiothreitol, 1 mM AEBSF, 1× Complete protease inhibitor cocktail (Roche, Basel, Switzerland). Protein concentrations were measured by BCA (Pierce Biotechnology, Rockford, Ill., United States). Protein extracts were subjected to polyacrylamide gel electrophoresis using the 4%-12% NuPAGE gel system (Invitrogen), transferred to PVDF (Millipore, Billerica, Mass., United States) membranes, and immunoblotted using antibodies that specifically recognize SIRT1 (DB083; Delta Biolabs, Gilroy, Calif., United States, and 05-707; Upstate, Charlottesville, Va., United States), E-cadherin (Transduction Laboratories 610182; BD Biosciences, San Diego, Calif., United States), hMLH1 (551091; BD Biosciences), cyclin D1 (556470; BD Biosciences), p27Kip1 (Transduction Laboratories K25020; BD Biosciences), the unphosphorylated (active) form of β-catenin (05-665; Upstate), and phospho-GSK3β (05-643; Upstate). On the same blot, β-actin (Sigma, St. Louis, Mo., United States) was probed to ensure equal loading.

Reporter assays were performed as described previously using the β-catenin-responsive TCF reporter [28] and the cyclin D1 reporter. Briefly, prior to transfection, RKO cells were plated in six-well tissue culture dishes and grown until they reached 80%-90% confluence. Cells were transfected with 500 ng of pGL3-OT, a TCF-LEF-responsive reporter, or pGL3-OF, a negative control with a mutated TCF-LEF binding site in combination with 10 ng of pRL-CMV vector. Twenty-four hours post-transfection, cells were treated with either vehicle (DMSO) control or with 700 μM SPT for 24 hours. According to the manufacturer's instructions, Firefly luciferase activity was measured via a luminometer (BD Biosciences) and normalized to the Renilla luciferase activities by using the Dual Luciferase Reporter System (Promega, Madison, Wis., United States).

ChIP. ChIP analysis was performed as previously described [4] with a few modifications. Antibodies to SIRT1 (05-707 and 07-313), acetyl-sH3-K9 (07-352), and acetyl-H4-K16 (07-329) were obtained from Upstate. Antibodies to SIRT1 were also obtained from Delta Biolabs (DB083). Primers (Forward: AGCCGCGTCTGGTTCTAGT; Reverse: GGAGGCTGCAGGGCTG) were designed for the SFRP1 promoter spanning −163 to +12 relative to the transcription start site (+1) and were amplified by PCR under linear conditions. Enrichment was calculated as the ratio between the net intensity of the bound SFRP1 sample divided by the input and the vector control sample divided by the input. Primers for E-cadherin were (Forward: TAGAGGGTCACCGCGTCTATG) and (Reverse: GGGTGCGTGGCTGCAGCCAGG), which encompass a CAAT signal.

MSP and bisulfite sequencing. MSP and bisulfite sequencing were performed as previously described [28,38] on DNA from MDA-MB-231 cells both transiently and stably infected with control vector or RNAi retrovirus.

### Example 2

In addition to the ability to transcriptionally re-activate DNA hypermethylated and silenced genes in cancer cells by inhibiting SIRT1 with siRNA treatment, dominant negative expression, and the small molecules splitomycin and nicotinamide, the same effects have been achieved with two compounds described herein, 2-chloro-5,6,7,8,9,10-hexahydrocyclohepta[b]indole-6-carboxamide and (S)-6-chloro-2,3,4,9-tetrahydro-1H-carbazole-1-carboxamide, which have been shown to give potent inhibition of SIRT1 deactylase activity. This has been accomplished for the SFRP1 and 2 genes in human breast cancer cells (line H231) and for SFRP1 in human colon cancer cells (line RKO).

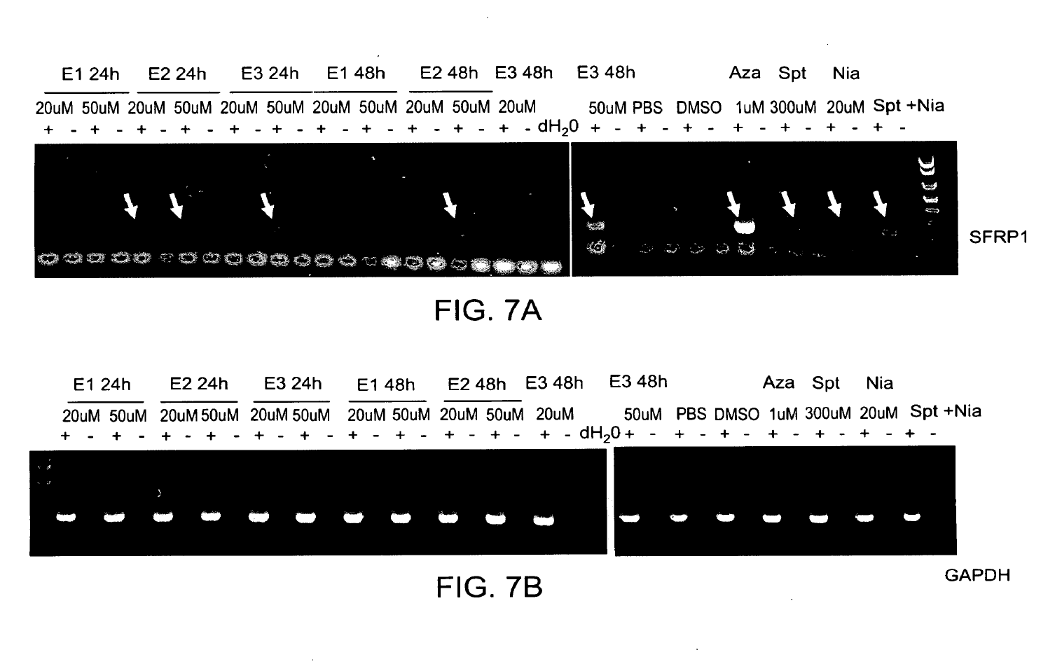

The results from experiments performed in RKO cells are shown in FIG. 7 which presents RT-PCR results for SFRP1. Note that the inactive compound, (R)-6-chloro-2,3,4,9-tetrahydro-1H-carbazole-1-carboxamide produces no specific band (white arrow) at doses of 20 and 50 μM for either 24 or 48 hours, while both of these doses for 2-chloro-5,6,7,8,9,10-hexahydrocyclohepta[b]indole-6-carboxamide and (S)-6-chloro-2,3,4,9-tetrahydro-1H-carbazole-1-carboxamide produce the band at both time points. The expression seemed stronger than for 300 μM splitomycin (Spt) or 20 mM nicotinamide (Nia) when these latter two drugs were used alone or in combination, but not as strong as the expression induced by 1 μM of the DNA demethylating agent, deoxy-aza-cytidine (Aza). The bottom PCR results show expression of GAPDH as a loading control. Experiments were carried out as essentially described above.

A number of embodiments of the invention have been described. Nevertheless, it will be understood that various modifications may be made without departing from the spirit and scope of the invention. Accordingly, other embodiments are within the scope of the following claims.

### REFERENCES

The following specific references, also incorporated by reference, are indicated in the examples and discussion above by a number in brackets.
1. Herman J G, Baylin S B (2003) Gene silencing in cancer in association with promoter hypermethylation. N Engl J Med 349: 2042-2054.
2. Jones P A, Baylin S B (2002) The fundamental role of epigenetic events in cancer. Nat Rev Genet. 3: 415-428.
3. Jones P A, Laird P W (1999) Cancer epigenetics comes of age. Nat Genet. 21: 163-167.
4. Fahrner J A, Eguchi S, Herman J G, Baylin S B (2002) Dependence of histone modifications and gene expression on DNA hypermethylation in cancer. Cancer Res 62: 7213-7218.
5. Cameron E E, Bachman K E, Myohanen S, Herman J G, Baylin S B (1999) Synergy of demethylation and histone deacetylase inhibition in the re-expression of genes silenced in cancer. Nat Genet. 21: 103-107.
6. Suzuki H, Gabrielson E, Chen W, Anbazhagan R, van Engeland M, et al. (2002) A genomic screen for genes upregulated by demethylation and histone deacetylase inhibition in human colorectal cancer. Nat Genet. 31: 141-149.
7. Blander G, Guarente L (2004) The Sir2 family of protein deacetylases. Annu Rev Biochem 73: 417-435.
8. Picard F, Kurtev M, Chung N, Topark-Ngarm A, Senawong T, et al. (2004) Sirt1 promotes fat mobilization in white adipocytes by repressing PPAR-gamma. Nature 429: 771-776.
9. Senawong T, Peterson V J, Leid M (2005) BCL11A-dependent recruitment of SIRT1 to a promoter template in mammalian cells results in histone deacetylation and transcriptional repression. Arch Biochem Biophys 434: 316-325.
10. Senawong T, Peterson V J, Avram D, Shepherd D M, Frye R A, et al. (2003) Involvement of the histone deacetylase SIRT1 in chicken ovalbumin upstream promoter transcription factor (COUP-TF)-interacting protein 2-mediated transcriptional repression. J Biol Chem 278: 43041-43050.
11. Vaquero A, Scher M, Lee D, Erdjument-Bromage H, Tempst P, et al. (2004) Human SirT1 interacts with histone H1 and promotes formation of facultative heterochromatin. Mol Cell 16: 93-105.
12. Bitterman K J, Anderson R M, Cohen H Y, Latorre-Esteves M, Sinclair D A (2002) Inhibition of silencing and accelerated aging by nicotinamide, a putative negative regulator of yeast sir2 and human SIRT1. J Biol Chem 277: 45099-45107.
13. Bedalov A, Gatbonton T, Irvine W P, Gottschling D E, Simon J A (2001) Identification of a small molecule inhibitor of Sir2p. Proc Natl Acad Sci USA 98: 15113-15118.
14. Avalos J L, Bever K M, Wolberger C (2005) Mechanism of sirtuin inhibition by nicotinamide: Altering the NAD(+) cosubstrate specificity of a Sir2 enzyme. Mol Cell 17: 855-868.
15. Kelly W K, O'Connor O A, Krug L M, Chiao J H, Heaney M, et al. (2005) Phase I study of an oral histone deacetylase inhibitor, suberoylanilide hydroxamic acid, in patients with advanced cancer. J Clin Oncol 23: 3923-3931.
16. Drummond D C, Noble C O, Kirpotin D B, Guo Z, Scott G K, et al. (2005) Clinical development of histone deacetylase inhibitors as anticancer agents. Annu Rev Pharmacol Toxicol 45: 495-528.
17. Olaharski A J, Rine J, Marshall B L, Babiarz J, Zhang L, et al. (2005) The favoring agent dihydrocoumarin reverses epigenetic silencing and inhibits sirtuin deacetylases. PLoS Genet. 1: e77.
18. Guarente L (2000) Sir2 links chromatin silencing, metabolism, and aging. Genes Dev 14: 1021-1026.
19. Guarente L, Kenyon C (2000) Genetic pathways that regulate ageing in model organisms. Nature 408: 255-262.
20. Nemoto S, Fergusson M M, Finkel T (2004) Nutrient availability regulates SIRT1 through a forkhead-dependent pathway. Science 306: 2105-2108.
21. Vaziri H, Dessain S K, Ng Eaton E, Imai S I, Frye R A, et al. (2001) hSIR2(SIRT1) functions as an NAD-dependent p53 deacetylase. Cell 107: 149-159.
22. Luo J, Nikolaev A Y, Imai S, Chen D, Su F, et al. (2001) Negative control of p53 by Sir2alpha promotes cell survival under stress. Cell 107: 137-148.
23. Langley E, Pearson M, Faretta M, Bauer U M, Frye R A, et al. (2002) Human SIR2 deacetylates p53 and antagonizes PML/p53-induced cellular senescence. EMBO J 21: 2383-2396.
24. Brunet A, Sweeney L B, Sturgill J F, Chua K F, Greer P L, et al. (2004) Stress-dependent regulation of FOXO transcription factors by the SIRT1 deacetylase. Science 303: 2011-2015.
25. Giannakou M E, Partridge L (2004) The interaction between FOXO and SIRT1: tipping the balance towards survival. Trends Cell Biol 14: 408-412.
26. Gottschling D E (2000) Gene silencing: Two faces of SIR2. Curr Biol 10: R708-R711.
27. Furuyama T, Banerjee R, Breen T R, Harte P J (2004) SIR2 is required for polycomb silencing and is associated with an E(Z) histone methyltransferase complex. Curr Biol 14: 1812-1821.
28. Suzuki H, Watkins D N, Jair K W, Schuebel K E, Markowitz S D, et al. (2004) Epigenetic inactivation of SFRP genes allows constitutive WNT signaling in colorectal cancer. Nat Genet 36: 417-422.
29. Corn P G, Heath E I, Heitmiller R, Fogt F, Forastiere A A, et al. (2001) Frequent hypermethylation of the 5′ CpG island of E-cadherin in esophageal adenocarcinoma. Clin Cancer Res 7: 2765-2769.
30. Strathdee G (2002) Epigenetic versus genetic alterations in the inactivation of E-cadherin. Semin Cancer Biol 12: 373-379.
31. Corn P G, Smith B D, Ruckdeschel E S, Douglas D, Baylin S B, et al. (2000) E-cadherin expression is silenced by 5′ CpG island methylation in acute leukemia. Clin Cancer Res 6: 4243-4248.
32. Herman J G, Umar A, Polyak K, Graff J R, Ahuja N, et al. (1998) Incidence and functional consequences of hMLH1 promoter hypermethylation in colorectal carcinoma. Proc Natl Acad Sci USA 95: 6870-6875.
33. Kane M F, Loda M, Gaida G M, Lipman J, Mishra R, et al. (1997) Methylation of the hMLH1 promoter correlates with lack of expression of hMLH1 in sporadic colon tumors and mismatch repair-defective human tumor cell lines. Cancer Res 57: 808-811.
34. Akiyama Y, Watkins N, Suzuki H, Jair K W, van Engeland M, et al. (2003) GATA-4 and GATA-5 transcription factor genes and potential downstream antitumor target genes are epigenetically silenced in colorectal and gastric cancer. Mol Cell Biol 23: 8429-8439.
35. Graff J R, Gabrielson E, Fujii H, Baylin S B, Herman J G (2000) Methylation patterns of the E-cadherin 5′ CpG island are unstable and reflect the dynamic, heterogeneous loss of E-cadherin expression during metastatic progression. J Biol Chem 275: 2727-2732.
36. Anderson R M, Bitterman K J, Wood J G, Medvedik O, Sinclair D A (2003) Nicotinamide and PNC1 govern lifespan extension by calorie restriction in Saccharomyces cerevisiae. Nature 423: 181-185.
37. Posakony J, Hirao M, Stevens S, Simon J A, Bedalov A (2004) Inhibitors of Sir2: Evaluation of splitomicin analogues. J Med Chem 47: 2635-2644.
38. Herman J G, Graff J R, Myohanen S, Nelkin B D, Baylin S B (1996) Methylation-specific PCR: A novel PCR assay for methylation status of CpG islands. Proc Natl Acad Sci USA 93: 9821-9826.
39. Imai S, Armstrong C M, Kaeberlein M, Guarente L (2000) Transcriptional silencing and longevity protein Sir2 is an NAD-dependent histone deacetylase. Nature 403: 795-800.
40. Suka N, Luo K, Grunstein M (2002) Sir2p and Sas2p opposingly regulate acetylation of yeast histone H4 lysine 16 and spreading of heterochromatin. Nat Genet 32: 378-383.
41. Kimura A, Umehara T, Horikoshi M (2002) Chromosomal gradient of histone acetylation established by Sas2p and Sir2p functions as a shield against gene silencing. Nat Genet 32: 370-377.
42. Shankaranarayana G D, Motamedi M R, Moazed D, Grewal S I (2003) Sir2 regulates histone H3 lysine 9 methylation and heterochromatin assembly in fission yeast. Curr Biol 13: 1240-1246.
43. Korinek V, Barker N, Morin P J, van Wichen D, de Weger R, et al. (1997) Constitutive transcriptional activation by a beta-catenin-Tcf complex in APC^{-/-} colon carcinoma. Science 275: 1784-1787.
44. Morin P J, Sparks A B, Korinek V, Barker N, Clevers H, et al. (1997) Activation of beta-catenin-Tcf signaling in colon cancer by mutations in beta-catenin or APC. Science 275: 1787-1790.
45. Tetsu O, McCormick F (1999) Beta-catenin regulates expression of cyclin D1 in colon carcinoma cells. Nature 398: 422-426.
46. Pruitt K, Pestell R G, Der C J (2000) Ras inactivation of the retinoblastoma pathway by distinct mechanisms in NIH 3T3 fibroblast and RIE-1 epithelial cells. J Biol Chem 275: 40916-40924.
47. Motta M C, Divecha N, Lemieux M, Kamel C, Chen D, et al. (2004) Mammalian SIRT1 represses forkhead transcription factors. Cell 116: 551-563.
48. Huguet E L, McMahon J A, McMahon A P, Bicknell R, Harris A L (1994) Differential expression of human Wnt genes 2,3,4, and 7B in human breast cell lines and normal and disease states of human breast tissue. Cancer Res 54: 2615-2621.
49. Yeung F, Hoberg J E, Ramsey C S, Keller M D, Jones D R, et al. (2004) Modulation of NF-kappaB-dependent transcription and cell survival by the SIRT1 deacetylase. EMBO J 23: 2369-2380.
50. Kuzmichev A, Margueron R, Vaquero A, Preissner T S, Scher M, et al. (2005) Composition and histone substrates of polycomb repressive group complexes change during cellular differentiation. Proc Natl Acad Sci USA 102: 1859-1864.
51. Chen W Y, Wang D H, Yen R C, Luo J, Gu W, et al. (2005) Tumor suppressor HIC1 directly regulates SIRT1 to modulate p53-dependent DNA-damage responses. Cell 123: 437-448.
52. Wood J G, Rogina B, Lavu S, Howitz K, Helfand S L, et al. (2004) Sirtuin activators mimic caloric restriction and delay ageing in metazoans. Nature 430: 686-689.
53. Ford J, Jiang M, Milner J (2005) Cancer-specific functions of SIRT1 enable human epithelial cancer cell growth and survival. Cancer Res 65: 10457-10463.
54. Toyota M, Issa J P (1999) CpG island methylator phenotypes in aging and cancer. Semin Cancer Biol 9: 349-357.
55. Fraga M F, Ballestar E, Villar-Garea A, Boix-Chornet M, Espada J, et al. (2005) Loss of acetylation at Lys16 and trimethylation at Lys20 of histone H4 is a common hallmark of human cancer. Nat Genet 37: 391-400.
56. Esteller M, Guo M, Moreno V, Peinado M A, Capella G, et al. (2002) Hypermethylation-associated inactivation of the cellular retinol-binding-protein 1 gene in human cancer. Cancer Res 62: 5902-5905.

1. A method of activating methylation silenced genes in a subject comprising administering a histone deacetylase (HDAC) inhibitor.

2. The method of claim 1, wherein activating the genes comprises increased gene expression.

3. The method of claim 1, wherein the HDAC inhibitor is administered in combination with one or more agents.

4. The method of claim 1, wherein the genes that are silenced by methylation are methylated in the promoter region.

5. The method of claim 1, wherein the methylation is hypermethylation.

6. The method of claim 1, wherein the subject is suffering from a proliferative disease or disorder.

7. The method of claim 6, wherein the proliferative disease or disorder is selected from a neoplasia, myelofibrosis, or proliferative diabetic retinopathy.

8. The method of claim 7, wherein the neoplasia is a cancer.

9. The method of claim 8, wherein the cancer is selected from the group consisting of: breast, ovarian, liver, lung, and prostate.

10. The method of claim 8, wherein the cancer comprises genes that are silenced by methylation.

11. The method of claim 10, wherein the genes are tumor suppressor genes.

12. The method of claim 11, wherein the tumor suppressor genes are selected from the group consisting of: secreted frizzled related proteins, p53, E-cadherin, mismatch repair genes, and cellular retinol binding protein-1.

13. A method of activating methylation silenced genes in a subject comprising administering a histone deacetylase (HDAC) inhibitor in combination with one or more agents.

14. The method of claim 13, wherein gene activation comprises increased gene expression.

15. A method of treating a proliferative disease or disorder comprising administering a histone deacetylase (HDAC) inhibitor in combination with one or more agents.

16. The method of any one of claims 1-15, wherein at least one of the one or more agents is an inhibitor of epigenetic silencing.

17. The method of claims 1, 13 or 15, wherein the HDAC inhibitor is a class III HDAC inhibitor.

18. The method of claim 17, wherein the class III HDAC inhibitor is a SIRT1 inhibitor.

19. A method of treating a proliferative disease or disorder comprising administering a SIRT1 inhibitor in combination with one or more agents, wherein at least one of the one or more agents is an inhibitor of epigenetic silencing.

20. The method of claim 17, wherein the class III HDAC inhibitor is selected from an siRNA, a dsRNA, a shRNA, a ribozyme, an antisense nucleic acid, a retroviral inhibitor, an adenoviral inhibitor, or a small molecule inhibitor.

21. The method of claim 20, wherein the siRNA inhibits expression of SIRT1.

22. A siRNA that inhibits expression of SIRT1 in a cell.

23. A siRNA according to claim 21 or claim 22 which comprises a contiguous sequence of 10-30 bp from the sequence of SEQ ID NO: 1.

24. A siRNA according to claim 23 that is between 19 and 25 bp in length.

25. A siRNA according to claim 24 comprising SEQ ID NO: 3, SEQ ID NO: 4 or SEQ ID NO: 5

26. The method of claim 1, 13, 15, or 19 wherein at least one of the one or more agents is an agent that promotes demethylation.

27. The method of claim 26, wherein at least one of the one or more agents is a HDAC inhibitor.

28. The method of claim 27, wherein the HDAC inhibitor is selected from the group consisting of an inhibitor of the class of: HDAC I, HDAC II and HDACIII.

29. The method of claim 26, wherein the agent is selected from: 5-azadeoxycytodine, nicotinamide, splitomicin, and trichostatin-A.

30. The method of claim 1, 13, 15, or 19 or 26, wherein at least one of the one or more agents is a chemotherapeutic agent.

31. A method of identifying a SIRT1 inhibitor comprising:
administering a candidate compound to a cell with one or more genes that are silenced by methylation in vitro; and
determining whether gene expression in increased in said cell;
wherein increased gene expression compared to untreated cells identifies a SIRT1 inhibitor.

32. The method of claim 31, wherein the SIRT1 inhibitor does not affect gene methylation.

33. The method of any one of claims 13-32, wherein the proliferative disease or disorder is selected from a neoplasia, myelofibrosis, or proliferative diabetic retinopathy.

34. The method of claim 33, wherein the neoplasia is a cancer.

35. The method of claim 34, wherein the cancer is selected from the group consisting of: breast, ovarian, liver, lung, and prostate cancer.

36. The method of claim 34, wherein the cancer comprises genes that are silenced by methylation.

37. The method of claim 36, wherein the genes are tumor suppressor genes.

38. The method of claim 37, wherein the tumor suppressor genes are selected from the group consisting of: secreted frizzled related proteins, p53, E-cadherin, mismatch repair genes, and cellular retinol binding protein-1.

39. A pharmaceutical composition comprising a siRNA according to any one of claims 22-25 and a pharmaceutically acceptable excipient.

40. A pharmaceutical composition comprising a SIRT1 inhibitor according to any one of claims 31-32 and a pharmaceutically acceptable excipient

41. A kit for use in a method of activating methylation silenced genes in a subject comprising administering a histone deacetylase (HDAC) inhibitor according to any one of claims 1-12 and instructions for use.

42. A kit for use in the method of activating methylation silenced genes in a subject comprising administering a histone deacetylase (HDAC) inhibitor in combination with one or more agents and instructions for use.

43. A kit for use in a method of treating a proliferative disease or disorder comprising administering a histone deacetylase (HDAC) inhibitor in combination with one or more agents according to any one of claims 15-18 and instructions for use.
